SEQUENCE LISTING (12 sequences)

SEQ ID NO: 1 - DNA, 16 bp, synthetic construct
  source 1..16: mol_type = other DNA
  modified_base order(1..3,14..16): mod_base = OTHER; note = Locked Nucleic Acid
  modified_base order(1,16): mod_base = OTHER; note = 5-methylcytosine
  modified_base 1..16: mod_base = OTHER; note = phosphorothioate bond
ctagttcactgaatgc

SEQ ID NO: 2 - DNA, 16 bp, synthetic construct
  source 1..16: mol_type = other DNA
  modified_base order(1..3,14..16): mod_base = OTHER; note = Locked Nucleic Acid
  modified_base 1..16: mod_base = OTHER; note = phosphorothioate bond
atagtcactctggtga

SEQ ID NO: 3 - DNA, 20 bp, synthetic construct
  source 1..20: mol_type = other DNA
  modified_base order(1..5,16..20): mod_base = OTHER; note = 2'-O-MOE-RNA
  modified_base order(4,18): mod_base = OTHER; note = thymine
  modified_base order(5,17): mod_base = OTHER; note = 5-methylcytosine
  modified_base 1..20: mod_base = OTHER; note = phosphorothioate bond
gggtcagctgccaatgctag

SEQ ID NO: 4 - RNA, 16 nt, synthetic construct
  source 1..16: mol_type = other RNA
  modified_base order(1..3,14..16): mod_base = OTHER; note = 2'-O-Me-RNA
  modified_base order(1..4,13..16): mod_base = OTHER; note = phosphorothioate bond
  modified_base 1: mod_base = OTHER; note = choleterol binding at 5' end
gcattcagtgaactag

SEQ ID NO: 5 - RNA, 16 nt, synthetic construct
  source 1..16: mol_type = other RNA
  modified_base order(1..3,14..16): mod_base = OTHER; note = 2'-O-Me-RNA
  modified_base order(1..4,13..16): mod_base = OTHER; note = phosphorothioate bond
gcattcagtgaactag

SEQ ID NO: 6 - RNA, 20 nt, synthetic construct
  source 1..20: mol_type = other RNA
  modified_base order(1..5,7,11..20): mod_base = OTHER; note = 2'-O-Me-RNA
  modified_base order(6,8..10): mod_base = OTHER; note = 2'-fluoro-RNA
  modified_base order(1..3,18..20): mod_base = OTHER; note = phosphorothioate bond
catttaatcctcactctaaa

SEQ ID NO: 7 - RNA, 23 nt, synthetic construct
  source 1..23: mol_type = other RNA
  modified_base 1: mod_base = OTHER; note = 5'-(E)-Vinylphosphonate(VP)-2'-O-Me-RNA
  modified_base order(2,6,8..9,14,16): mod_base = OTHER; note = 2'-fluoro-RNA
  modified_base order(3..5,7,10..13,15,17..23): mod_base = OTHER; note = 2'-O-Me-RNA
  modified_base order(1..3,21..23): mod_base = OTHER; note = phosphorothioate bond
tttagagtgaggattaaaatgag

SEQ ID NO: 8 - DNA, 16 bp, synthetic construct
  source 1..16: mol_type = other DNA
  modified_base order(1..3,14..16): mod_base = OTHER; note = Locked Nucleic Acid
  modified_base 14: mod_base = OTHER; note = 5-methylcytosine
  modified_base 1..16: mod_base = OTHER; note = phosphorothioate bond
atttccaaattcactt

SEQ ID NO: 9 - DNA, 25 bp, synthetic construct
  source 1..25: mol_type = other DNA
  modified_base 1..25: mod_base = OTHER; note = morpholino nucleic acid
  modified_base 1..25: mod_base = OTHER; note = phosphorodiamidate bond
ggccaaacctcggcttacctgaaat

SEQ ID NO: 10 - DNA, 21 bp, synthetic construct
  source 1..21: mol_type = other DNA
atccagcagtcagaaagcaaa

SEQ ID NO: 11 - DNA, 21 bp, synthetic construct
  source 1..21: mol_type = other DNA
cagccatccatttctgtaagg

SEQ ID NO: 12 - DNA, 25 bp, synthetic construct
  source 1..25: mol_type = other DNA
  modified_base order(2..4,9,19..21): mod_base = OTHER; note = uracil
  modified_base 1: mod_base = OTHER; note = choleterol binding at 5' end
  modified_base order(1..4,22..25): mod_base = OTHER; note = phosphorothioate bond
  modified_base order(1..3,23..25): mod_base = OTHER; note = 2'-O-Me-RNA
atttcaggtaagccgaggtttggcc

(19) United States
(12) Patent Application Publication
(10) Pub. No.: US 2026/0347843 A1
(43) Pub. Date: Nov. 5, 2026

(54) NERVOUS SYSTEM DELIVERY ENHANCER

(71) Applicants: INSTITUTE OF SCIENCE TOKYO, Tokyo (JP); NIIGATA UNIVERSITY, Niigata-shi, Niigata (JP)

(72) Inventors: Takanori YOKOTA, Tokyo (JP); Kotaro YOSHIOKA, Tokyo (JP); Tetsuya NAGATA, Tokyo (JP); Rintaro HARA, Tokyo (JP); Hironaka IGARASHI, Niigata-shi, Niigata (JP)

(73) Assignees: INSTITUTE OF SCIENCE TOKYO, Tokyo (JP); NIIGATA UNIVERSITY, Niigata-shi, Niigata (JP)

(21) Appl. No.: 19/115,991

(22) PCT Filed: Sep. 29, 2023
(86) PCT No.: PCT/JP2023/035565

§ 371 Date: Mar. 27, 2025

(30) Foreign Application Priority Data
Sep. 30, 2022 (JP) 2022-158781

(51) Int. Cl. C12N 15/113; A61K 31/7105; A61K 47/22

(52) U.S. Cl. CPC C12N 15/113; A61K 31/7105; A61K 47/22; C12N 2310/11; C12N 2310/341

(57) ABSTRACT

A problem is to provide a new method for enhancing the efficiency of delivery of a drug, such as a nucleic acid medicine, to a nervous system. Provided is an agent for promoting delivery, consisting of an agent for promoting the function of Aquaporin-4 or an agent for modifying the function of Aquaporin-4, and intended to promote delivery of a drug to the central nervous system and/or the peripheral nervous system.

### TECHNICAL FIELD

The present invention relates to: an agent for promoting delivery of a drug to the central nervous system and/or the peripheral nervous system; and a pharmaceutical composition for treating the central nervous system disease or the peripheral nervous system disease.

### BACKGROUND ART

In recent years, an oligonucleotide has been drawing attention in the ongoing development of a pharmaceutical product called a nucleic acid medicine, and in particular, development of a nucleic acid medicine utilizing the antisense method is actively carried out from the viewpoint of high selectivity on the target gene and low toxicity. The antisense method is a method comprising selectively modifying or inhibiting the expression of a protein encoded by a target gene or the activity of miRNA by introducing into a cell an oligonucleotide complementary to a target sense strand that is a partial sequence of mRNA or miRNA transcribed from the target gene (antisense oligonucleotide, herein often referred to as “ASO”).

As a nucleic acid utilizing the antisense method, the present inventors have developed a double-stranded nucleic acid complex (heteroduplex oligonucleotide, HDO) in which an antisense oligonucleotide and a complementary strand thereto are annealed (Patent Literature 1 and 2, Non-Patent Literature 1 and 2). A double-stranded nucleic acid complex provides an epoch-making technology that has a high antisense effect.

In the medication for the central nervous system disease or the peripheral nervous system disease, the efficiency of delivery of a drug to the nervous system is often a problem. There is a demand for a method of increasing the efficiency of delivery of a drug to the central nervous system and/or the peripheral nervous system to improve a therapeutic effect.

### CITATION LIST

### Patent Literature

Patent Literature 1: WO2013/089283
Patent Literature 2: WO2014/192310
Patent Literature 3: WO2017/150704

### Non-Patent Literature

Non-Patent Literature 1: Nishina K, et. al., “DNA/RNA heteroduplex oligonucleotide for highly efficient gene silencing”, Nature Communications, 2015, 6:7969.
Non-Patent Literature 2: Asami Y, et al., “Drug delivery system of therapeutic oligonucleotides”, Drug Discoveries & Therapeutics. 2016; 10 (5): 256-262.
Non-Patent Literature 3: Huber V. J. et al., NeuroReport, 2018, 29 (9): 697-703

### SUMMARY OF INVENTION

### Technical Problem

A problem is to provide a new method for improving the efficiency of delivery of a drug, such as a nucleic acid medicine, to a nervous system.

### Solution to Problem

Transport of water into and out of a cell in a nervous system is adjusted by a transmembrane water channel, such as an aquaporin (AQP). Aquaporin-4 (AQP4) is a member of the aquaporin family having very high water selectivity, and is a main AQP involved in transport of water in a nervous system. In addition, in the central nervous system including the brain and the spinal cord, AQP4 is present in a large amount, particularly in an end-feet membrane of an astrocyte in contact with a basement membrane.

The periarterial space/perivenous space that are present between a blood vessel and a brain tissue that are distributed in the brain are called Virchow-Robin spaces. In recent years, it has been suggested that the degradation products of proteins and other waste products in cells are transported via a Virchow-Robin space so as to be reabsorbed into a vena cava, and that they flow into a cerebrospinal fluid (CSF) in a cerebral ventricle. This transport is similar to the transport by a lymphatic system. The mechanism that discharges a waste product out of the brain via the periarterial space/perivenous space is called a glymphatic system.

The above-described Patent Literature 3 (WO2017/150704) discloses that promoting the water transport by AQP4 in the brain to promote the discharging function of the glymphatic system leads to reduction of the accumulation of amyloid-β in the brain.

The present inventors have tried activating a glymphatic system by administering, to the cerebral ventricle of a mouse, an agent for promoting the function of AQP4 simultaneously with a nucleic acid medicine such as an antisense nucleic acid or siRNA. As a result, the inventors have discovered that the amount of the nucleic acid medicine detected in the nervous system after the administration was increased dramatically, and that the inhibitory effect against a target gene was improved markedly. This result is a surprising effect, unpredictable from the discharge-promoting effect on amyloid-β.

The present invention is based on the above-described findings and provides the following.

(1) An agent for promoting delivery of a drug to the central nervous system and/or the peripheral nervous system, consisting of an agent for promoting the function of Aquaporin-4 or an agent for modifying the function of Aquaporin-4.

(2) The agent for promoting delivery of (1), wherein the agent for promoting the function of Aquaporin-4 or the agent for modifying the function of Aquaporin-4 is a compound represented by the following formula (I) or a derivative thereof, or a salt of any of the compound and the salt;

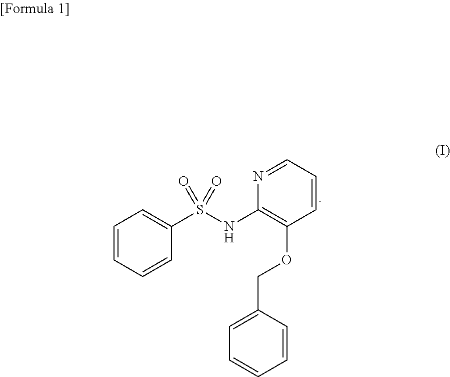 [SMILES: O=S(=O)(Nc1ncccc1OCc1ccccc1)c1ccccc1]

(3) The agent for promoting delivery of (2), wherein the derivative is represented by the following formula (III):

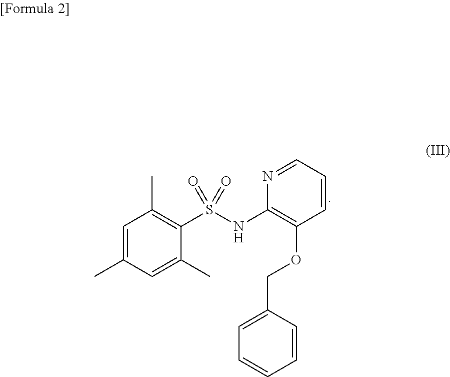 [SMILES: Cc1cc(C)c(S(=O)(=O)Nc2ncccc2OCc2ccccc2)c(C)c1]

(4) The agent for promoting delivery of (2) or (3), wherein the compound or the derivative is bound to a carrier molecule.

(5) The agent for promoting delivery of (4), wherein the carrier molecule is a protein of 1 kDa or more, a polymer, a lipid molecule, or a contrast agent.

(6) The agent for promoting delivery of (5), wherein the protein is an albumin, a lipoprotein, or an antibody or an antibody fragment.

(7) The agent for promoting delivery of (5), wherein the polymer comprises polyethylene glycol (PEG) or a PEG-grafted polymer.

(8) The agent for promoting delivery of (5) or (7), wherein the polymer or the lipid molecule forms a microbubble, liposome, or micelle.

(9) The agent for promoting delivery of any one of (1) to (8), wherein the drug is administered by intrathecal administration, transnasal administration, intravenous administration, subcutaneous administration, intraperitoneal administration, oral administration, inhalation administration, or intramuscular administration.

(10) The agent for promoting delivery of (9), wherein the intrathecal administration is intraventricular administration, suboccipital puncture, or lumbar puncture.

(11) The agent for promoting delivery of any one of (1) to (10), wherein the drug is administered with a shunt, indwelling catheter, or subcutaneous port.

(12) The agent for promoting delivery of any one of (1) to (11), which is administered simultaneously with the drug, or administered before or after the drug.

(13) The agent for promoting delivery of any one of (1) to (12), which is administered by intrathecal administration, transnasal administration, intravenous administration, subcutaneous administration, intraperitoneal administration, oral administration, inhalation administration, or intramuscular administration.

(14) A pharmaceutical composition for treating the central nervous system disease or the peripheral nervous system disease, comprising a therapeutically effective amount of a drug, and the agent for promoting delivery of any one of (1) to (8).

(15) The pharmaceutical composition of (14), which is administered by intrathecal administration, transnasal administration, intravenous administration, subcutaneous administration, intraperitoneal administration, oral administration, inhalation administration, or intramuscular administration.

(16) The pharmaceutical composition of (15), wherein the intrathecal administration is intraventricular administration, suboccipital puncture, or lumbar puncture.

(17) The pharmaceutical composition of any one of (14) to (16), which is administered with a shunt, indwelling catheter, or subcutaneous port.

(18) The agent for promoting delivery of any one of (1) to (13), or the pharmaceutical composition of any one of (14) to (17), wherein the drug is a nucleic acid medicine, peptide, small molecule compound, viral vector, cellular medicine, nanoparticle, liposome, micelle, or exosome.

(19) The agent for promoting delivery or the pharmaceutical composition of (18), wherein the peptide is a natural peptide or a non-natural peptide.

(20) The agent for promoting delivery or the pharmaceutical composition of (18), wherein the peptide is a cyclic peptide.

(21) The agent for promoting delivery or the pharmaceutical composition of (18), wherein the peptide is a protein formulation.

(22) The agent for promoting delivery to a brain or the pharmaceutical composition of (18), wherein the peptide is an antibody or an antibody fragment.

(25) The agent for promoting delivery or the pharmaceutical composition of (18), wherein the nucleic acid medicine is selected from the group consisting of an antisense nucleic acid, heteronucleic acid, siRNA, shRNA, miRNA, mRNA, lncRNA, plasmid DNA, aptamer, decoy, bait nucleic acid, ribozyme, and nucleic acid vector.

(26) The agent for promoting delivery or the pharmaceutical composition of (18), wherein the nucleic acid medicine comprises a nucleic acid molecule which is capable of hybridizing to at least part of a target gene or a transcriptional product thereof, and has an antisense effect for the target gene or the transcriptional product thereof.

(27) The agent for promoting delivery or the pharmaceutical composition of (26), wherein the nucleic acid molecule is 12 to 30 bases in length.

(28) The agent for promoting delivery or the pharmaceutical composition of (26) or (27), wherein the nucleic acid molecule comprises any one or more selected from the group consisting of deoxyribonucleoside, 2′-modified nucleoside, 5′-modified nucleoside, and bridged nucleoside.

(29) The agent for promoting delivery or the pharmaceutical composition of any one of (26) to (28), wherein the nucleic acid molecule is a mixmer.

(30) The agent for promoting delivery or the pharmaceutical composition of any one of (26) to (29), wherein the nucleic acid molecule comprises a morpholino nucleic acid or all of the nucleic acid in the nucleic acid molecule consists of a morpholino nucleic acid.

(31) The agent for promoting delivery or the pharmaceutical composition of any one of (26) to (30), wherein the nucleic acid molecule is a gapmer.

(32) The agent for promoting delivery or the pharmaceutical composition of (31), wherein the nucleic acid molecule comprises:
(1) a central region which comprises at least three consecutive deoxyribonucleosides,
(2) a 5′-wing region which comprises a non-natural nucleoside, positioned on the 5′ end side of the central region, and
(3) a 3′-wing region which comprises a non-natural nucleoside, positioned on the 3′ end side of the central region.

(33) The agent for promoting delivery or the pharmaceutical composition of (32), comprising a 2′-modified nucleoside at: the terminal base position adjacent to the central region in the 5′-wing region, the 2nd base position from 5′ side of the central region, and/or the 8th base position from 5′ side of the central region.

(34) The agent for promoting delivery or the pharmaceutical composition of (32), wherein, in the nucleic acid molecule, the 5′-wing region and the 3′-wing region comprise a bridged nucleoside and/or a 2′-modified nucleoside.

(35) The agent for promoting delivery or the pharmaceutical composition of (34), wherein the bridged nucleoside is selected from the group consisting of LNA nucleoside, 2′,4′-BNA^{NC} nucleoside, cEt BNA nucleoside, ENA nucleoside, AmNA nucleoside, GuNA nucleoside, scpBNA nucleoside, scpBNA2 nucleoside, and BANA3 nucleoside.

(36) The agent for promoting delivery or the pharmaceutical composition of (34) or (35), wherein the 2′-modified nucleoside is 2′-O-methyl-modified nucleoside, 2′-O-methoxyethyl-modified nucleoside, 2′-O-[2-(N-methylcarbamoyl)ethyl]-modified nucleoside, or 2′-fluoro-modified nucleoside.

(37) The agent for promoting delivery or the pharmaceutical composition of any one of (26) to (36), wherein all or a part of the internucleoside linkage in the nucleic acid molecule is a modified internucleoside linkage.

(38) The agent for promoting delivery or the pharmaceutical composition of (37), wherein the modified internucleoside linkage is a phosphorothioate linkage.

(39) The agent for promoting delivery or the pharmaceutical composition of any one of (26) to (38), wherein the nucleic acid molecule comprises a modified nucleobase.

(40) The agent for promoting delivery or the pharmaceutical composition of any one of (26) to (39), wherein the nucleic acid molecule is capable of inducing steric blocking, splicing regulation, reduction of expression, increase of expression, and/or base editing in the target gene or the transcriptional product thereof.

(41) The agent for promoting delivery or the pharmaceutical composition of any one of (26) to (40), wherein the nucleic acid medicine consists of a double-stranded nucleic acid complex comprising a first nucleic acid strand consisting of the nucleic acid molecule and a second nucleic acid strand comprising a base sequence complementary to the first nucleic acid strand.

(42) The agent for promoting delivery or the pharmaceutical composition of (41), wherein the second nucleic acid strand is at least 8 bases in length.

(43) The agent for promoting delivery or the pharmaceutical composition of (41) or (42), wherein the second nucleic acid strand comprises any one or more selected from the group consisting of deoxyribonucleoside, 2′-modified nucleoside, 5′-modified nucleoside, and bridged nucleoside.

(44) The agent for promoting delivery or the pharmaceutical composition of any one of (41) to (43), wherein the second nucleic acid strand comprises a noncomplementary base, and/or an insertion sequence and/or deletion of one or more bases, relative to the first nucleic acid strand.

(45) The agent for promoting delivery or the pharmaceutical composition of (44), wherein the second nucleic acid strand comprises one to three of the noncomplementary bases.

(46) The agent for promoting delivery or the pharmaceutical composition of (44) or (45), wherein the insertion sequence consists of 1 to 8 bases.

(47) The agent for promoting delivery or the pharmaceutical composition of any one of (44) to (46), wherein the deletion consists of 1 to 4 consecutive bases.

(48) The agent for promoting delivery or the pharmaceutical composition of any one of (41) to (47), wherein the second nucleic acid strand comprises at least one overhang region positioned on the 5′ end side and/or 3′ end side of a region consisting of the base sequence complementary to the first nucleic acid strand.

(49) The agent for promoting delivery or the pharmaceutical composition of (48), wherein the overhang region is 1 to 30 bases in length.

(50) The agent for promoting delivery or the pharmaceutical composition of any one of (41) to (49), wherein the second nucleic acid strand is bound to a functional moiety.

(51) The agent for promoting delivery or the pharmaceutical composition of (50), wherein the functional moiety is a lipid or a peptide.

(52) The agent for promoting delivery or the pharmaceutical composition of (51), wherein the peptide is an antibody.

(53) The agent for promoting delivery or the pharmaceutical composition of (51), wherein the lipid is selected from the group consisting of cholesterol or an analog thereof, tocopherol or an analog thereof, folic acid, phosphatidylethanolamine, and a substituted or unsubstituted C₁₆₋₃₀ alkyl group.

(54) The agent for promoting delivery or the pharmaceutical composition of any one of (41) to (53), wherein the first nucleic acid strand and the second nucleic acid strand are bound via a linker.

(55) The agent for promoting delivery or the pharmaceutical composition of (54), wherein the linker is a cleavable or uncleavable linker.

(56) The agent for promoting delivery or the pharmaceutical composition of (54) or (55), wherein the linker comprises a group represented by the following formula (IV):

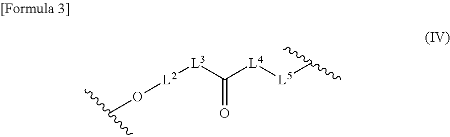

(wherein
L² represents a substituted or unsubstituted C₁₋₁₂ alkylene group, a substituted or unsubstituted C₃-C₈ cycloalkylene group, —(CH₂)₂—O—(CH₂)₂—O—(CH₂)₂—O—(CH₂)₃—, or CH(CH₂—OH)—CH₂—O—(CH₂)₂—O—(CH₂)₂—O—(CH₂)₂—O—(CH₂)₃—;
L³ represents —NH— or a bond;
L⁴ represents a substituted or unsubstituted C₁₋₁₂ alkylene group, a substituted or unsubstituted C₃-C₈ cycloalkylene group, —(CH₂)₂—[O—(CH₂)₂]ₘ—, or a bond, wherein m is an integer of 1 to 25; and
L⁵ represents —NH—(C═O)—, —(C═O)—, or a bond).

(57) The agent for promoting delivery or the pharmaceutical composition of any one of (54) to (56), wherein the linker comprises a nucleic acid, polyether group, and/or alkylamino group.

(58) The agent for promoting delivery or the pharmaceutical composition of (57), wherein the nucleic acid consists of one or 2 to 10 nucleosides and/or non-natural nucleosides linked via internucleoside linkages.

(59) The agent for promoting delivery or the pharmaceutical composition of (57), wherein the polyether group is a polyethylene glycol group or a triethylene glycol group.

(60) The agent for promoting delivery or the pharmaceutical composition of (57), wherein the alkylamino group is a hexylamino group.

(61) The agent for promoting delivery or the pharmaceutical composition of any one of (41) to (60), wherein all or a part of the internucleoside linkages in the second nucleic acid strand are modified internucleoside linkages.

(62) The agent for promoting delivery or the pharmaceutical composition of (61), wherein the modified internucleoside linkages are phosphorothioate linkages.

(63) The agent for promoting delivery or the pharmaceutical composition of (18), wherein the nucleic acid medicine is siRNA.

(64) The agent for promoting delivery or the pharmaceutical composition of (63), wherein the siRNA comprises any one or more selected from the group consisting of a deoxyribonucleoside, 2′-modified nucleoside, 5′-modified nucleoside, bridged nucleoside, and modified internucleoside linkage.

(65) The agent for promoting delivery or the pharmaceutical composition of (63) or (64), wherein the siRNA is bound to a functional moiety.

(66) The agent for promoting delivery or the pharmaceutical composition of (65), wherein the functional moiety is a lipid or a peptide.

(67) The agent for promoting delivery or the pharmaceutical composition of (66), wherein the lipid is selected from the group consisting of cholesterol or an analog thereof, tocopherol or an analog thereof, folic acid, phosphatidylethanolamine, and a substituted or unsubstituted C₁₆₋₃₀ alkyl group.

(68) The agent for promoting delivery or the pharmaceutical composition of (63), wherein the nucleic acid medicine comprises two siRNAs, and the guide strands comprised in the two siRNAs are linked to each other via a linker.

(69) The agent for promoting delivery or the pharmaceutical composition of (68), wherein the linker comprises a nucleic acid, polyether group, and/or alkylamino group.

(70) The agent for promoting delivery or the pharmaceutical composition of any one of (1) to (69), wherein the central nervous system is selected from the group consisting of cerebral cortex, basal ganglia, cerebral white matter, diencephalon, brainstem, cerebellum, and spinal cord.

(71) The agent for promoting delivery or the pharmaceutical composition of (70), wherein the central nervous system is selected from the group consisting of frontal lobe, temporal lobe, hippocampus, parahippocampal gyrus, parietal lobe, occipital lobe, striatum, globus pallidus, claustrum, thalamus, subthalamic nucleus, midbrain, substantia nigra, pons, medulla oblongata, cerebellar cortex, cerebellar nucleus, cervical cord, thoracic cord, and lumbar cord.

(72) The agent for promoting delivery or the pharmaceutical composition of any one of (1) to (69), wherein the peripheral nervous system is selected from the group consisting of a ventral root of the spinal cord, dorsal root, 1st to 12th cranial nerves, cauda equina, and dorsal root ganglion.

(73) The agent for promoting delivery or the pharmaceutical composition of any one of (1) to (72), wherein 0.01 mg to 10 g of the agent for promoting delivery is administered to a subject.

The present specification encompasses the contents disclosed in Japanese Patent Application No. 2022-158781, which is the basis of the priority of the present application.

### Advantageous Effects of Invention

The present invention provides an agent for promoting delivery for improving the efficiency of delivery of a drug to a nervous system.

### BRIEF DESCRIPTION OF DRAWINGS

FIG. 1 shows the structures of various natural nucleotides or non-natural nucleotides.

FIG. 2 shows the structures of various bridged nucleic acids.

FIG. 3 shows the amount of delivery of a Malat1-targeting ASO to the central nervous system and the target gene suppression effect. FIG. 3A shows the results obtained by measuring the ASO concentration in the hippocampus at 3 hours or 7 days after the intraventricular administration. FIG. 3B shows the Malat1 RNA expression level in the hippocampus at 7 days after the intraventricular administration. “ASO only” represents the ASO single administration group, and “ASO+TGN-073” represents the ASO/TGN-073 coadministration group. The error bars indicate standard errors.

FIG. 4 shows: the amount of delivery of the Malat1-targeting ASO to the central nervous system, in which the ASO was intraventricularly administered at various concentrations; and the target gene suppression effect in the central nervous system. “ASO only” represents the ASO single administration group, and “ASO+TGN-073” represents the ASO/TGN-073 coadministration group. The error bars indicate standard errors.

FIG. 5 shows the Mapt mRNA expression level in the hippocampus cord at 7 days after the intraventricular administration of the Mapt-targeting ASO. “ASO only” represents the ASO single administration group, and “ASO+TGN-073” represents the ASO/TGN-073 coadministration group. The error bars indicate standard errors.

FIG. 6 shows a scoring system used for behavioral evaluation.

FIG. 7 shows the results of evaluation of the central nervous system toxicity of the Malat1-targeting ASO. The Figure shows the acute tolerability scores at 30 minutes, 1 hour, and 2 hours after the intraventricular administration. “TGN-073 only” represents the TGN-073 single administration group, “ASO only” represents the ASO single administration group, and “ASO+TGN-073” represents the ASO/TGN-073 coadministration group. The error bars indicate standard errors.

FIG. 8 shows the results of evaluation of the motor function in an open-field test at 1 hour after the intraventricular administration. FIG. 8A shows the maximum moving speed.

FIG. 8B shows the moving time. “TGN-073 only” represents the TGN-073 single administration group, “ASO only” represents the ASO single administration group, and “ASO+TGN-073” represents the ASO/TGN-073 coadministration group. The error bars indicate standard errors.

FIG. 9 shows the Malat1 RNA expression level in the hippocampus at 7 days after the intraventricular administration of 2′-O-MOE-modified ASO targeted at the Malat1 gene. “PBS” represents the PBS administration group as a negative control, “ASO only” represents the ASO single administration group, and “ASO+TGN-073” represents the ASO/TGN-073 coadministration group. The error bars indicate standard errors.

FIG. 10 shows the structures of nucleic acids used in Example 5.

FIG. 11 shows the ASO concentration (the amount of delivery to the central nervous system) and the target gene suppression effect in the central nervous system when a single-stranded or double-stranded nucleic acid agent targeted at the Malat1 gene was intraventricularly administered. FIG. 11A shows the results obtained by measuring the ASO concentration in the hippocampus at 7 days after the intraventricular administration. FIG. 11B shows the Malat1 RNA expression level in the hippocampus at 7 days after the intraventricular administration. In the figure, whether TGN-073 was intraventricularly coadministered together with a nucleic acid agent is denoted by +/−. The error bars indicate standard errors.

FIG. 12 shows the target gene suppression effect when an siRNA targeted at the Sod1 gene was intraventricularly administered. “PBS” represents the PBS administration group as a negative control, “siRNA only” represents the siRNA single administration group, and “siRNA+TGN-073” represents the siRNA/TGN-073 coadministration group. The error bars indicate standard errors.

FIG. 13 shows the ASO concentration (the amount of delivery to the central nervous system) and the target gene suppression effect in the central nervous system when TGN-073-mes, which was a derivative of TGN-073, was intraventricularly administered together with ASO. FIG. 13A shows the results obtained by measuring the ASO concentration in the hippocampus at 7 days after the intraventricular administration. FIG. 13B shows the Mapt mRNA expression level in the hippocampus at 7 days after the intraventricular administration. “PBS” represents the PBS administration group as a negative control, “TGN-073-mes only” represents the TGN-073-mes single administration group, and “ASO only” represents the ASO single administration group, and “ASO+TGN-073-mes” represents the ASO/TGN-073-mes coadministration group. The error bars indicate standard errors.

FIG. 14 shows the skipping efficiency of exon 23 in the brain at 7 days after the intraventricular administration of a phosphorodiamidate morpholino oligomer (PMO) targeted at exon 23/intron 23 of dystrophin pre-mRNA. FIG. 14A shows the skipping efficiency in the hippocampus. FIG. 14B shows the skipping efficiency in the striatum. FIG. 14C shows the skipping efficiency in the cortex. The error bars indicate standard errors.

FIG. 15 shows the structures of nucleic acids used in Example 8.

FIG. 16 shows the skipping efficiency of exon 23 in the brain at 7 days after the intraventricular administration of a double-stranded nucleic acid complex comprising PMO, and targeted at exon 23/intron 23 of dystrophin pre-mRNA. FIG. 16A shows the skipping efficiency in the hippocampus. FIG. 16B shows the skipping efficiency in the striatum.

FIG. 16C shows the skipping efficiency in the cortex. The error bars indicate standard errors.

FIG. 17 shows the amount of delivery of a Malat1-targeting ASO to the lumbar cord portion and the target gene suppression effect. FIG. 17A shows the results obtained by measuring the ASO concentration in the lumbar cord portion at 7 days after the intraventricular administration. FIG. 17B shows the Malat1 RNA expression level in the lumbar cord portion at 7 days after the intraventricular administration. “PBS” represents the PBS administration group as a negative control, “ASO only” represents the ASO single administration group, and “ASO+TGN-073” represents the ASO/TGN-073 coadministration group. The error bars indicate standard errors.

FIG. 18 shows the amount of delivery of a Malat1-targeting ASO to the central nervous system, in which the ASO was intrathecally administered. FIG. 18A shows the ASO concentration in the spinal cord tissue. FIG. 18B shows the ASO concentration in the left hippocampus. “ASO only” represents the ASO single administration group, and “ASO+TGN-073” represents the ASO/TGN-073 coadministration group. The error bars indicate standard errors.

FIG. 19 shows the amount of delivery of a VHH antibody to the central nervous system, in which the antibody was intraventricularly administered. FIG. 19A shows the VHH antibody concentration in the left hippocampus. FIG. 19B shows the VHH antibody concentration in the left occipital cortex. FIG. 19C shows the VHH antibody concentration in the left basal ganglia. “VHH only” represents the VHH antibody single administration group, and “VHH+TGN-073” represents the VHH antibody/TGN-073 coadministration group. The error bars indicate standard errors.

FIG. 20 shows the amount of delivery of an IgG antibody to the central nervous system, in which the antibody was intraventricularly administered. “IgG only” represents the IgG antibody single administration group, and “IgG+TGN-073” represents the IgG antibody/TGN-073 coadministration group. The error bars indicate standard errors.

FIG. 21 shows the results obtained by observing, in a hippocampus level coronal image, the fluorescence of GFP protein expressed from the AAV9 vector administered intraventricularly. “AAV only” represents the AAV single administration group, and “AAV+TGN-073” represents the AAV/TGN-073 coadministration group.

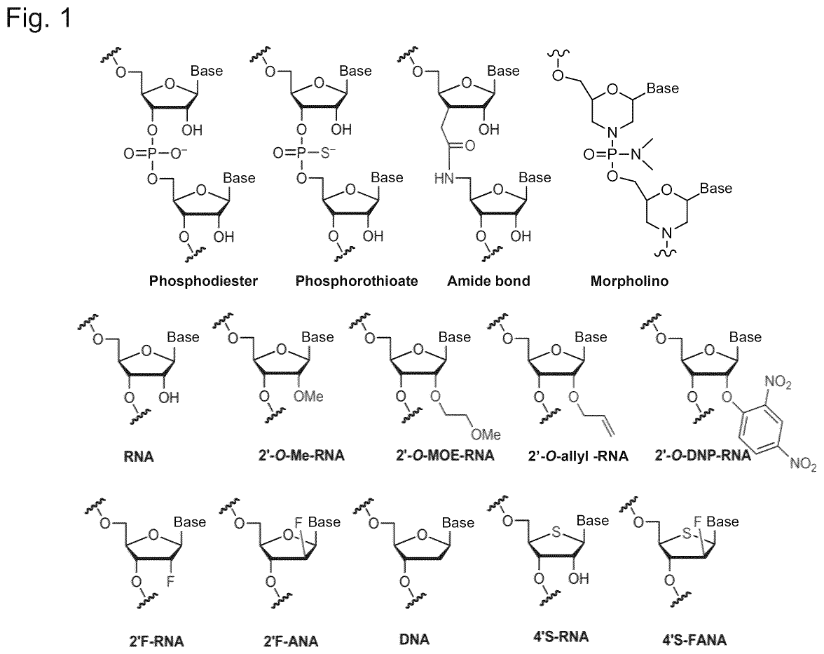

### DESCRIPTION OF EMBODIMENTS

### <1. Agent for Promoting Delivery>

A first aspect of the present invention is an agent for promoting delivery. An agent for promoting delivery of the present invention consists of an agent for promoting the function of Aquaporin-4 or an agent for modifying the function of Aquaporin-4. Using an agent for promoting delivery of the present invention in combination with a drug, such as a nucleic acid medicine, peptide, or small molecule compound, makes it possible to improve the efficiency of delivery of the drug to the central nervous system and/or the peripheral nervous system.

### DEFINITION OF TERMS

A “transcriptional product” of a target gene means herein any RNA that is synthesized by an RNA polymerase. Specifically, mRNA transcribed from a target gene (comprising, e.g., mature mRNA, mRNA precursor, and mRNA without base modification), non-coding RNA (ncRNA) such as miRNA, long non-coding RNA (lncRNA), and natural antisense RNA can be included.

A “target gene” means herein a gene wherein the expression amount of a transcriptional product or a translation product thereof can be reduced or increased by the antisense effect of the nucleic acid molecule or the double-stranded nucleic acid complex; a gene wherein the function of a transcriptional product or a translation product thereof can be inhibited by the effect; or a gene for which steric blocking, splicing regulation (e.g., splicing switching, exon skipping, and exon inclusion), base editing, or RNA editing can be induced by the effect. There is no particular restriction on the kind of target gene. Examples thereof include a gene which is derived from an organism into which a nucleic acid strand, a nucleic acid molecule, or a double-stranded nucleic acid complex is to be introduced, a gene whose expression is increased in various diseases (e.g., central nervous system diseases and peripheral nervous system diseases), and a gene that is expressed in vivo, such as a nervous system. Specific examples thereof include a scavenger receptor B1 (herein often referred to as “SR-B1”) gene, and a metastasis associated lung adenocarcinoma transcript 1 (herein often referred to as “Malat1”) gene, a microtubule-associated protein tau (herein often referred to as “Mapt”) gene, β-secretase 1 (herein often referred to as “BACE1”) gene, a histone deacetylase 2 (herein often referred to as “Hdac2”) gene, a DMPK (dystrophia myotonica-protein kinase) gene, and a dystrophin gene.

A “target transcriptional product” means herein a transcriptional product that is a direct target of the nucleic acid molecule or the double-stranded nucleic acid complex, and a “transcriptional product of a target gene” is also a target transcriptional product. The base sequence information for target transcriptional products and target genes can be obtained from publicly known databases, such as the database of NCBI (The U.S. National Center for Biotechnology Information).

An “antisense nucleic acid” herein refers to a single-stranded nucleic acid molecule that comprises a base sequence capable of hybridizing (i.e., complementary) to at least a part of a target transcriptional product (mainly, a transcriptional product of a target gene), and can produce an antisense effect on the target transcriptional product. In addition, an “antisense oligonucleotide” herein means an antisense nucleic acid composed of oligonucleotides. The “antisense nucleic acid” or “antisense oligonucleotide” is herein often referred to as “ASO”. The nucleic acid molecule in the present invention or the first nucleic acid strand of the double-stranded nucleic acid complex functions as ASO, and its target region may comprise 3′UTR, 5′UTR, exon, intron, coding region, translation initiation region, translation termination region, or any other nucleic acid region. The target region of a target transcriptional product may be at least 8 bases in length, e.g., 10 to 35 bases in length, 12 to 25 bases in length, 13 to 20 bases in length, 14 to 19 bases in length, 15 to 18 bases in length, 13 to 22 bases in length, 16 to 22 bases in length, or 16 to 20 bases in length.

An “antisense effect” herein means any effect produced by hybridization of ASO to a target transcriptional product (e.g., RNA sense strand), such as an effect of regulating expression or editing of the target transcriptional product. The phrase “regulating expression or editing of a target transcriptional product” includes suppression or reduction of the expression of a target gene or the expression amount of a target transcriptional product (“expression amount of a target transcriptional product” is herein often referred to as “expression level of a target transcriptional product”), inhibition of translation, RNA editing, base editing, splicing regulation or a splicing function modifying effect (e.g., splicing switching, exon inclusion, and exon skipping), steric blocking, or degradation of a transcriptional product. For example, in the case of post-transcriptional inhibition of a target gene, when an RNA oligonucleotide is introduced into a cell as ASO, the ASO forms a partial double strand by annealing to mRNA which is a transcriptional product of a target gene. This partial double strand serves as a cover to prevent translation by ribosomes, so as to inhibit the expression of the target protein encoded by the target gene at the translation level (steric blocking). On the other hand, when an oligonucleotide comprising DNA is introduced into a cell as ASO, a partial DNA-RNA heteroduplex is formed. This heteroduplex structure is recognized by RNase H, and as a result mRNA of the target gene is degraded and the expression of the protein encoded by the target gene is inhibited at the expression level. In addition, an antisense effect can also be produced for an intron in an mRNA precursor as a target. Furthermore, an antisense effect can also be produced for miRNA as a target. In this case, as a result of functional inhibition of the miRNA, the expression of the gene whose expression is normally regulated by the miRNA may be increased. In one embodiment, expression regulation of a target transcriptional product may be a decrease in the amount of a target transcriptional product.

The antisense effect can be measured, e.g., as follows: a subject nucleic acid compound is administered to a subject (e.g., a mouse); and, e.g., after several days (e.g., after 2 to 7 days), a measurement is made of the expression amount of a target gene or the level (amount) of the target transcriptional product (e.g., the amount of mRNA, the amount of RNA such as microRNA, the amount of cDNA, the amount of protein, or the like), wherein the expression of the target gene is regulated by the antisense effect provided by the subject nucleic acid compound.

For example, when the expression amount measured of the target gene or the level measured of the target transcriptional product is decreased by at least 10%, at least 20%, at least 25%, at least 30%, or at least 40%, compared with a negative control (e.g., vehicle administration), the measurement shows that the subject nucleic acid compound can produce an antisense effect (e.g., a decrease in the amount of the target transcriptional product).

The number, kind, and position of a non-natural nucleotide in a nucleic acid strand may influence the antisense effect and the like provided by the nucleic acid strand, the nucleic acid molecule, or the nucleic acid complex. The choice of a modification may vary depending on the sequence of a target gene or the like, but those skilled in the art can determine a suitable embodiment by referring to the descriptions in the literature related to the antisense method (e.g., WO 2007/143315, WO 2008/043753, and WO 2008/049085). Furthermore, when the antisense effect of a nucleic acid complex after the modification is measured and the obtained measured value is not significantly lower than the measured value of the nucleic acid complex before the modification (e.g., when the measured value obtained after the modification is 70% or more, 80% or more, or 90% or more of the measured value of the nucleic acid complex before the modification), a relevant modification may be evaluated.

A “translation product of the target gene” means herein any polypeptide or protein that is synthesized by translation of a target transcriptional product or a transcriptional product of a target gene which is a direct target of a nucleic acid molecule or double-stranded nucleic acid complex.

Herein, “decoy” refers to a nucleic acid having the sequence of a binding site of a transcription factor (e.g., NF-kB) or a similar sequence, and the nucleic acid is introduced as a “decoy” into a cell to inhibit the function of a transcription factor (inhibit transcription in the case of a transcription activator or promote transcription in the case of a transcription repressor). A decoy nucleic acid can be easily designed based on information about a binding sequence of a target transcription factor.

Herein, “bait” refers to a nucleic acid molecule that specifically binds to a particular target molecule in a cell and modifies a function of the target molecule. A target that interacts with a bait is also referred to as a “prey”.

The term “nucleic acid” or “nucleic acid molecule” used herein may refer to a nucleotide or nucleoside of a monomer, and may mean an oligonucleotide composed of a plurality of monomers or a plurality of nucleosides linked via an internucleoside linkage, or means a polynucleotide in the case of a polymer. A “natural nucleic acid” refers to a naturally-occurring nucleic acid. Examples of the natural nucleic acid include a natural nucleoside as described below, natural nucleotide, and the like. A “non-natural nucleic acid” or “artificial nucleic acid” refers to any nucleic acid other than a natural nucleic acid. Examples of the non-natural nucleic acid or the artificial nucleic acid include a non-natural nucleoside as described below, a non-natural nucleotide, and the like.

A “nucleic acid strand” or simply “strand” herein means two or more nucleosides linked via an internucleoside linkage, and may be, e.g., an oligonucleotide or a polynucleotide. A full-length strand or a partial length strand of a nucleic acid strand can be produced, e.g., by a chemical synthesis using an automated synthesizer, or by an enzymatic step using a polymerase, a ligase, or a restricted reaction. A nucleic acid strand may comprise a natural nucleotide and/or a non-natural nucleotide.

A “nucleoside” generally means a molecule consisting of a combination of a base and a sugar. The sugar moiety of a nucleoside is usually, but not limited to, composed of pentofuranosyl sugar, and specific examples thereof include ribose and deoxyribose. The base moiety of nucleoside (nucleobase) is usually a heterocyclic base moiety. Without limitation, examples thereof include adenine, cytosine, guanine, thymine, or uracil as well as other modified nucleobases (modified bases).

A “nucleotide” refers to a molecule in which a phosphate group is covalently bound to the sugar moiety of the nucleoside. In the case of a nucleotide comprising pentofuranosyl sugar, a phosphate group is usually linked to a hydroxyl group at the 2′, 3′, or 5′ position of the sugar.

An “oligonucleotide” refers to a linear oligomer formed by linking several to dozens of neighboring nucleotides via a covalent bond between a hydroxyl group and a phosphate group in the sugar moiety. Furthermore, a “polynucleotide” refers to a linear polymer formed by linking via covalent bonds dozens or more, preferably hundreds or more of nucleotides, namely more nucleotides than in an oligonucleotide. It is considered that the phosphate group generally forms an internucleoside linkage inside the structure of an oligonucleotide or a polynucleotide.

A “natural nucleoside” refers herein to a nucleoside that exists in nature. Examples thereof include a ribonucleoside consisting of a ribose and the aforementioned base such as adenine, cytosine, guanine, or uracil, or a deoxyribonucleoside consisting of a deoxyribose and the aforementioned base such as adenine, cytosine, guanine, or thymine. In this regard, a ribonucleoside found in RNA, and a deoxyribonucleoside found in DNA are herein often referred to as “RNA nucleoside” and “DNA nucleoside”, respectively.

A “natural nucleotide” means herein a nucleotide that exists in nature, namely a molecule in which a phosphate group is covalently bound to the sugar moiety of the aforementioned natural nucleoside. Examples thereof include a ribonucleotide which is known as a constituent of RNA, and in which a phosphate group is bound to a ribonucleoside, and a deoxyribonucleotide, which is known as a constituent of DNA, and in which a phosphate group is bound to a deoxyribonucleoside.

A “non-natural nucleotide” means herein any nucleotide other than a natural nucleotide. For example, it comprises a modified nucleotide and a nucleotide mimic. A “modified nucleotide” means herein a nucleotide having any one or more of a modified sugar moiety, a modified internucleoside linkage, and a modified nucleobase. The term “nucleotide mimic” herein comprises a structure used to substitute a nucleoside and a linkage at one or more positions in an oligomer compound. Examples of the nucleotide mimic include a peptide nucleic acid, and a morpholino nucleic acid (morpholino linked via —N(H)—C(═O)—O— or other non-phosphodiester linkages). The peptide nucleic acid (PNA) is a nucleotide mimic having a main chain in which N-(2-aminoethyl)glycine in place of a sugar is linked with an amide linkage. A nucleic acid strand comprising a non-natural oligonucleotide herein has, in many cases, preferable properties, such as enhanced cellular uptake, enhanced affinity for a target nucleic acid, increased stability in the presence of a nuclease, and an increase in inhibitory activity. Therefore, it is more preferable than a natural nucleotide.

A “non-natural nucleoside” means herein any nucleoside other than a natural nucleoside. For example, it comprises a modified nucleoside and a nucleoside mimic. A “modified nucleoside” means herein a nucleoside having a modified sugar moiety and/or a modified nucleobase.

A “mimic” refers herein to a functional group that substitutes a sugar, a nucleobase, and/or an internucleoside linkage. In general, a mimic is used in place of a sugar or a combination of a sugar-internucleoside linkage, and a nucleobase is maintained for hybridization to a target to be selected. The term “nucleoside mimic” used herein comprises a structure to be used for substituting a sugar at one or more positions of an oligomer compound, or substituting a sugar and a base, or substituting a bond between monomer subunits constituting an oligomer compound. An “oligomer compound” means a polymer composed of linked monomer subunits that can at least hybridize to a region of a nucleic acid molecule. Examples of a nucleoside mimic include a morpholino, cyclohexenyl, cyclohexyl, tetrahydropyranyl, bicyclic or tricyclic sugar mimic, such as a nucleoside mimic having a non-furanose sugar unit.

A “modified sugar” refers to a sugar in which a natural sugar moiety (i.e., sugar moiety found in DNA (2′-H) or RNA (2′-OH)) has undergone a substitution and/or any change. “Sugar modification” refers to substitution and/or any change from a natural sugar moiety. A nucleic acid strand may comprise, in some cases, one or more modified nucleosides comprising a modified sugar. A “sugar-modified nucleoside” means a nucleoside having a modified sugar moiety. Such a sugar-modified nucleoside can confer a beneficial biological property such as enhanced nuclease stability, increased binding affinity, or the like to a nucleic acid strand. In a specific embodiment, a nucleoside comprises a chemically modified ribofuranose ring moiety. Examples of a chemically modified ribofuranose ring comprise, but are not limited to, addition of a substituent (comprising 5′ and 2′ substituents), formation of a bicyclic nucleic acid (bridged nucleic acid, BNA) through bridge formation of a non-geminal ring atom, substitution of a ribosyl ring oxygen atom with S, N(R), or C(R1)(R2)(wherein R, R1, and R2 independently represent H, a C₁-C₁₂ alkyl, or a protecting group), and a combination thereof.

Examples of the sugar-modified nucleoside include, but are not limited to, a nucleoside comprising a substituent such as 5′-vinyl, and 5′-alkyl (e.g., 5′-methyl (R or S), 5′-ethyl (R or S)), 5′-allyl (R or S), 4′-S, 2′-F (2′-fluoro group), 2′-OCH₃ (2′-O-Me group or 2′-O-methyl group), 2′-O-[2-(N-methylcarbamoyl)ethyl] (2′-O-MCE group), and 2′-O-methoxyethyl(2′-O-MOE or 2-O (CH₂)₂OCH₃). A substituent at the 2′ position may be selected from allyl, amino, azide, thio, —O-allyl, —O—C₁-C₁₀ alkyl, —OCF₃, —O(CH₂)₂SCH₃, —O(CH₂)₂—O—N(Rm)(Rn), and O—CH₂—C(═O)—N(Rm)(Rn), wherein Rm and Rn are independently H or a substituted or unsubstituted C₁-C₁₀ alkyl. A “2′-modified sugar” means a furanosyl sugar modified at the 2′ position. A nucleoside comprising a 2′-modified sugar may be referred to as a “2′-modified nucleoside” or a “2′-sugar-modified nucleoside”. In addition, a “5′-modified sugar” means a furanosyl sugar modified at the 5′ position. A nucleoside comprising a 5′-modified sugar is referred to as a “5′-modified nucleoside” or a “5′-sugar-modified nucleoside”, and in particular, a deoxyribonucleoside comprising a 5′-modified sugar and a ribonucleoside comprising a 5′-modified sugar are respectively referred to as a “5′-modified deoxyribonucleoside” and a “5′-modified ribonucleoside”, e.g., to distinguish between them.

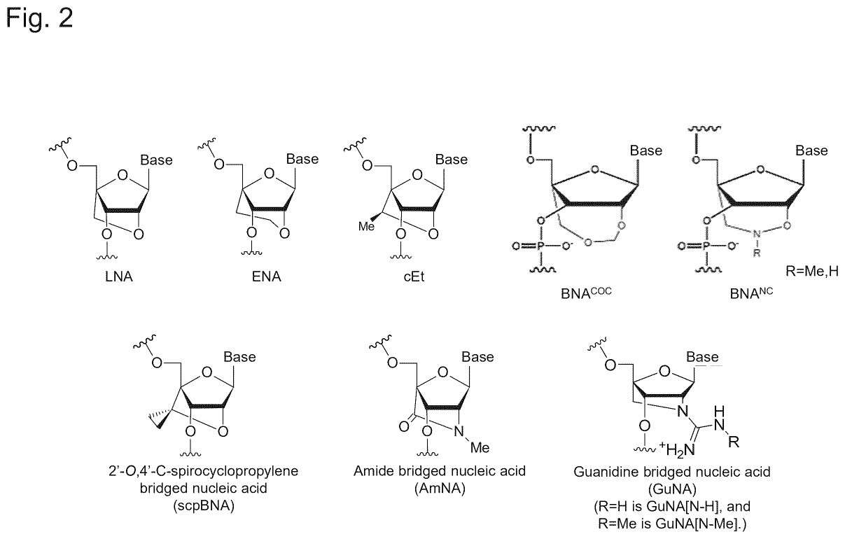

A “bicyclic nucleoside” refers to a modified nucleoside comprising a bicyclic sugar moiety. A nucleic acid comprising a bicyclic sugar moiety is commonly referred to as a bridged nucleic acid (BNA). A nucleoside comprising a bicyclic sugar moiety is sometimes referred to as a “bridged nucleoside”, “bridged-type non-natural nucleoside”, or “BNA nucleoside”. Some examples of a bridged nucleic acid are shown in FIG. 2.

A bicyclic sugar may be a sugar in which the carbon atom at the 2′ position and the carbon atom at the 4′ position are bridged with two or more atoms. Examples of a bicyclic sugar are known to those skilled in the art. A subgroup of nucleic acids comprising a bicyclic sugar (BNA) or of BNA nucleosides may be described as having a carbon atom at the 2′ position and a carbon atom at the 4′ position which are bridged with 4′-(CH₂)ₚ—O-2′, 4′-(CH₂)ₚ—CH₂-2′, 4′-(CH₂)ₚ—S-2′, 4′-(CH₂)ₚ—OCO-2′, 4′-(CH₂)ₙ—N(R₃)—O—(CH₂)ₘ-2′ [wherein p, m, and n represent integers from 1 to 4, from 0 to 2, and from 1 to 3, respectively; and R₃ represents a hydrogen atom, an alkyl group, an alkenyl group, a cycloalkyl group, an aryl group, an aralkyl group, an acyl group, a sulfonyl group, and a unit substituent (e.g., a fluorescently or chemiluminescently labeled molecule, a functional group with nucleic acid cleavage activity, and an intracellular or nuclear localization signal peptide)]. Furthermore, with respect to a BNA or a BNA nucleoside in a certain embodiment, in the OR₂ substituent of the carbon atom at the 3′ position and the OR₁ substituent of the carbon atom at the 5′ position, R₁ and R₂ are typically hydrogen atoms, but may be the same or different from each other, or may also be a protecting group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected by a protecting group for nucleic acid synthesis, or P(R₄)R₅ [wherein R₄ and R₅, may be the same or different from each other, and respectively represent a hydroxyl group, a hydroxyl group protected by a protecting group for nucleic acid synthesis, a mercapto group, a mercapto group protected by a protecting group for nucleic acid synthesis, an amino group, a C₁-C₅ alkoxy group, a C₁-C₅ alkylthio group, a C₁-C₆ cyanoalkoxy group, or an amino group substituted with a C₁-C₅ alkyl group]. Non-limiting examples of such BNA include methyleneoxy (4′-CH₂—O-2′) BNA (LNA, Locked Nucleic Acid®, also known as 2′,4′-BNA)(e.g., α-L-methyleneoxy (4′-CH₂—O-2′) BNA or β-D-methyleneoxy (4′-CH₂—O-2′) BNA), ethyleneoxy (4′-(CH₂)₂—O-2′) BNA (also known as ENA), β-D-thio (4′-CH₂—S-2′) BNA, aminooxy (4′-CH₂—O—N(R₃)-2′) BNA, oxyamino (4′-CH₂—N(R₃)—O-2′) BNA (also known as 2′,4′-BNA^{NC}; R=His 2′,4′-BNA^{NC} [N—H], R=Me is 2′,4′-BNA^{NC}[N-Me]), 2′,4′-BNAcoc, 3′-amino-2′,4′-BNA, 5′-methyl BNA, (4′-CH(CH₃)—O-2′) BNA (also known as cEt BNA), (4′-CH(CH₂OCH₃)—O-2′) BNA (also known as cMOE BNA), amide BNA (amide-bridged nucleic acid) or (4′-C(O)—N(R)-2′) BNA (R═H, or Me)(also known as AmNA; R═H in FIG. 2 is AmNA [N—H], R=Me is AmNA [N-Me]), guanidine BNA (also known as GuNA (e.g., R═H is GuNA [N—H], R=Me is GuNA [N-Me]) in FIG. 2), amine BNA (also known as 2′-Amino-LNA)(e.g., 3-(Bis(3-aminopropyl)amino) propanoyl substitution product), 2′-O,4′-C-spirocyclopropylene-bridged nucleic acid (also known as scpBNA), and other BNA known to those skilled in the art. Non-limiting examples of such BNA nucleoside include methyleneoxy (4′-CH₂—O-2′) BNA nucleoside (also known as LNA nucleoside or 2′,4′-BNA nucleoside)(e.g., α-L-methyleneoxy (4′-CH₂—O-2′) BNA nucleoside, β-D-methyleneoxy (4′-CH₂—O-2′) BNA nucleoside), ethyleneoxy (4′-(CH₂)₂—O-2′) BNA nucleoside (also known as ENA nucleoside), β-D-thio (4′-CH₂—S-2′) BNA nucleoside, aminooxy (4′-CH₂—O—N(R₃)-2′) BNA nucleoside, oxyamino (4′-CH₂—N(R₃)—O-2′) BNA nucleoside (also known as 2′,4′-BNA^{NC} nucleoside; R═H is 2′,4′-BNA^{NC} [N—H] nucleoside, and R=Me is 2′,4′-BNA^{NC} [N-Me] nucleoside), 2′,4′-BNAcoc nucleoside, 3′-amino-2′,4′-BNA nucleoside, 5′-methyl BNA nucleoside, (4′-CH(CH₃)—O-2′) BNA nucleoside (also known as cEt nucleoside), (4′-CH(CH₂OCH₃)—O-2′) BNA nucleoside (also known as cMOE nucleoside), amide BNA nucleoside or (4′-C(O)—N(R)-2′) BNA nucleoside (R═H, or Me)(also known as AmNA nucleoside; R═H in FIG. 2 is AmNA [N—H] nucleoside, and R=Me is AmNA [N-Me] nucleoside), guanidine BNA nucleoside (GuNA nucleoside (e.g., R═H in FIG. 2 is also known as GuNA [N—H] nucleoside, and R=Me is also known as GuNA [N-Me] nucleoside)), amine BNA nucleoside (also known as 2′-Amino-LNA nucleoside)(e.g., 3-(Bis(3-aminopropyl)amino) propanoyl-substituted nucleoside), 2′-O,4′-C-spirocyclopropylene-bridged nucleoside (also known as scpBNA nucleoside), and other BNA nucleosides known to those skilled in the art.

A “cationic nucleoside” herein is a modified nucleoside existing in cationic form, compared with a neutral form (such as the neutral form of ribonucleoside), at a pH (e.g., the physiological pH (approximately 7.4) of a human, a pH of a delivery site (e.g., an organelle, cell, tissue, organ, or organism), or the like). The cationic nucleoside may comprise one or more cationic modified groups at any position of a nucleoside. In one embodiment, the cationic nucleoside is 2′-Amino-LNA nucleoside (e.g., 3-(Bis(3-aminopropyl)amino) propanoyl-substituted nucleoside), aminoalkyl-modified nucleoside (e.g., 2′-O-methyl- and 4′-CH₂CH₂CH₂NH₂-substituted nucleoside), GuNA nucleoside (e.g., R═H in FIG. 2 is GuNA [N—H] nucleoside, and R=Me is GuNA [N-Me] nucleoside), or the like. A bicyclic nucleoside having a methyleneoxy (4′-CH₂—O-2′) bridge is also referred to as LNA nucleoside.

A “modified internucleoside linkage” means herein an internucleoside linkage that has a substitution or any change from a naturally occurring internucleoside linkage (i.e., phosphodiester linkage). A modified internucleoside linkage comprises an internucleoside linkage that comprises a phosphorus atom, and an internucleoside linkage that does not comprise a phosphorus atom. Typical examples of the phosphorus-comprising internucleoside linkage include, but are not limited to, a phosphodiester linkage, a phosphorothioate linkage, a phosphorodithioate linkage, a phosphotriester linkage (a methylphosphotriester linkage and an ethylphosphotriester linkage described in U.S. Pat. No. 5,955,599), an alkylphosphonate linkage (e.g., a methylphosphonate linkage described in U.S. Pat. Nos. 5,264,423 and 5,286,717, and a methoxypropylphosphonate linkage described in WO2015/168172), an alkylthiophosphonate linkage, a methylthiophosphonate linkage, a boranophosphate linkage, an internucleoside linkage comprising a cyclic guanidine moiety (e.g., a partial structure represented by the following Formula (V):

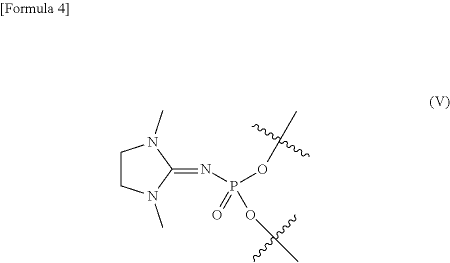 [SMILES: *OP(=O)(N=C1N(C)CCN1C)O*]

), an internucleoside linkage comprising a guanidine moiety substituted with one to four C₁₋₆ alkyl groups (e.g., a tetramethyl guanidine (TMG) moiety)(e.g., a partial structure represented by the following Formula (VI):

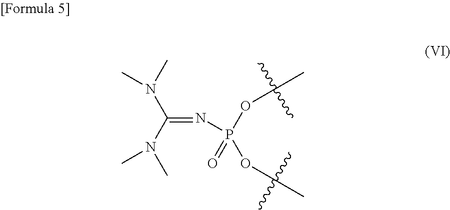 [SMILES: *OP(=O)(N=C(N(C)C)N(C)C)O*]

), and an internucleoside linkage and a phosphoramidate linkage that are to be used for a self-neutralizing nucleic acid (ZON) as described in WO2016/081600. A phosphorothioate linkage refers to an internucleoside linkage in which an unbridged oxygen atom in a phosphodiester linkage is substituted with a sulfur atom. A method for preparing a phosphorus-comprising and a phosphorus-free linkage is well known. It is preferable that a modified internucleoside linkage is a linkage having a higher resistance to a nuclease than a naturally occurring internucleoside linkage.

When an internucleoside linkage has a chiral center, the internucleoside linkage may be chirally controlled. The term “chirally controlled” means that a single diastereomer is present with respect to the chiral center, e.g., chirally bound phosphorus.

For example, the internucleoside linkage may be a phosphorothioate linkage chirally controlled to an Rp configuration or Sp configuration, an internucleoside linkage comprising a guanidine moiety substituted with one to four C₁₋₆ alkyl groups (e.g., a tetramethyl guanidine (TMG) moiety; see, e.g., Alexander A. Lomzov et al., Biochem Biophys Res Commun., 2019, 513 (4), 807-811), and/or an internucleoside linkage comprising a cyclic guanidine moiety. A phosphorothioate linkage chirally controlled to an Rp configuration or Sp configuration is also publicly known. A chirally controlled phosphorothioate linkage in the Sp configuration is known to be more stable than those in the Rp configuration, and chirally controlled ASOs in the Sp configuration are also known to promote target RNA cleavage by RNase H1, resulting in a more sustained response in vivo.

The term “nucleobase” or “base” used herein is a base component (heterocyclic moiety) constituting a nucleic acid. As the component, mainly adenine, guanine, cytosine, thymine, and uracil are known. The “nucleobase” or “base” herein encompasses both of a modified and an unmodified nucleobase (base), unless otherwise specified. Accordingly, a purine base may be any of a modified and an unmodified purine base, unless otherwise specified. In addition, a pyrimidine base may be any of a modified and an unmodified pyrimidine base, unless otherwise specified.

A “modified nucleobase” or a “modified base” means any nucleobase other than adenine, cytosine, guanine, thymine, or uracil. The term “unmodified nucleobase” or “unmodified base” (a natural nucleobase) means adenine (A) and guanine (G), which are purine bases, and thymine (T), cytosine (C), and uracil (U), which are pyrimidine bases. Examples of a modified nucleobase include, but are not limited to, hypoxanthine, 5-methylcytosine, 5-fluorocytosine, 5-bromocytosine, 5-iodocytosine, and N4-methylcytosine; N6-methyladenine or 8-bromoadenine; 2-thio-thymine; and N2-methylguanine or 8-bromoguanine.

The term “mixmer” refers to herein a nucleic acid strand that comprises natural nucleosides and non-natural nucleosides with periodically or randomly alternating segment lengths, and does not comprise four or more consecutive deoxyribonucleosides or ribonucleosides. Among mixmers, a mixmer in which the non-natural nucleoside is a bridged nucleoside, and the natural nucleoside is a deoxyribonucleoside is specifically referred to as a “BNA/DNA mixmer”. Among mixmers, a mixmer in which the non-natural nucleoside is a peptide nucleic acid and the natural nucleoside is a deoxyribonucleoside is specifically called a “peptide nucleic acid/DNA mixmer”. Among mixmers, a mixmer in which the non-natural nucleoside is a morpholino nucleic acid, and the natural nucleoside is a deoxyribonucleoside is specifically referred to as a “morpholino nucleic acid/DNA mixmer”. A mixmer is not restricted to comprise only two kinds of nucleosides. A mixmer may comprise any number of kinds of nucleosides irrespective of a natural or modified nucleoside, or a nucleoside mimic. For example, a mixmer may have one or two consecutive deoxyribonucleosides separated by a bridged nucleoside (e.g., an LNA nucleoside). The bridged nucleoside may further comprise a modified nucleobase (e.g., 5-methylcytosine).

Herein, the “gapmer” refers to a single-stranded nucleic acid that, in principle, comprises or consists of a “central region” (DNA gap region) and wing regions positioned directly at the 5′ end and 3′ end of the central region (the wing regions are referred to as a “5′-wing region” and a “3′-wing region” respectively). The central region in the gapmer comprises at least three or at least four consecutive deoxyribonucleosides. The 5′-wing region and the 3′-wing region each comprise at least one non-natural nucleoside. The position of the boundary between the central region (DNA gap region) and the 5′-wing region and the 3′-wing region in the gapmer can be easily determined from the sequence of the nucleoside by those skilled in the art. For example, the central region can be functionally defined as a region recognizable by RNase H (e.g., RNase H1). Here, the phrase “recognizable by RNase H” means that, when a gapmer is bound to a target RNA, the sequence that is in the target RNA, and paired with the gapmer can be cleaved by RNase H. Accordingly, in the gapmer, a region recognizable by RNase H can be regarded as the central region, and a region not recognizable by RNase H (more specifically, a region in which cleavage activity by RNase His not substantially detected under physiological conditions) can be regarded as a wing region (5′-wing region and 3′-wing region), so that the position of the boundary can be determined. Herein, in the 5′-wing region and the 3′-wing region, the terminal nucleosides adjacent to the central region are non-natural nucleosides (e.g., 2′-modified nucleosides or bridged nucleosides), and in the central region, the nucleoside adjacent to the 5′-wing region or the 3′-wing region is a deoxyribonucleoside or a sugar-modified form thereof. In one embodiment, in the central region, both of the nucleosides adjacent to the 5′-wing region and the 3′-wing region are deoxyribonucleosides. In the gapmer, the central region may comprise a modified nucleobase recognized by RNase H, e.g., 5-methylcytosine. The central region can also comprise a non-natural nucleoside such as a 2′-modified nucleoside and a 5′-modified nucleoside, except for two terminal nucleosides adjacent to the 5′-wing region and the 3′-wing region. In one embodiment, the central region may be constituted such that all the internucleoside linkages in the region paired in the target RNA can be cleaved by RNase H. Without limitation, a non-natural nucleoside comprised in the wing region usually has a higher bonding force to RNA than a natural nucleoside, and has high resistance to a nucleic acid-degrading enzyme (nuclease or the like). A non-natural nucleoside constituting the 5′-wing region and the 3′-wing region may be, e.g., a bridged nucleoside and/or a 2′-modified nucleoside. When a non-natural nucleoside constituting the wing region comprises or consists of a bridged nucleoside, the gapmer is specifically referred to as a “BNA/DNA gapmer”. The number of bridged nucleosides comprised in the 5′-wing region and the 3′-wing region may be at least one, e.g., two or three. The bridged nucleosides comprised in the 5′-wing region and the 3′-wing region may be consecutively or inconsecutively present in the 5′-wing region and the 3′-wing region. The bridged nucleoside may further comprise a modified nucleobase (e.g., 5-methylcytosine). The bridged nucleoside may be an LNA nucleoside or an ENA nucleoside. When the bridged nucleoside is an LNA nucleoside, the gapmer is referred to as an “LNA/DNA gapmer”. When the bridged nucleoside is an ENA nucleoside, the gapmer is referred to as an “ENA/DNA gapmer”. When a non-natural nucleoside constituting the 5′-wing region and the 3′-wing region comprises or consists of a peptide nucleic acid, the gapmer is specifically referred to as a “peptide nucleic acid gapmer”.

When a non-natural nucleoside constituting the 5′-wing region and the 3′-wing region comprises or consists of a morpholino nucleic acid, the gapmer is specifically referred to as a “morpholino nucleic acid gapmer”. When a non-natural nucleoside constituting the 5′-wing region and the 3′-wing region comprises or consists of a 2′-modified nucleoside, the 2′-modified group of the 2′-modified nucleoside may be a 2′-O-methyl group or a 2′-O-methoxyethyl group. The number of the 2′-modified nucleosides comprised in the 5′-wing region and the 3′-wing region may be at least one, e.g., two or three. The 2′-modified nucleosides comprised in the 5′-wing region and the 3′-wing region may be consecutively or inconsecutively present in the 5′-wing region and the 3′-wing region. The 2′-modified nucleoside may further comprise a modified nucleobase (e.g., 5-methylcytosine). When a non-natural nucleoside constituting the 5′-wing region and the 3′-wing region comprises or consists of a bridged nucleoside and a 2′-modified nucleoside, the non-natural nucleoside may be composed of a combination of two or more bridged nucleosides and/or 2′-modified nucleosides. In this regard, a nucleic acid strand having a wing region on only one of the 5′ end side or 3′ end side is referred to as a “hemi-gapmer” in the art, but herein, the hemi-gapmer is also encompassed in the gapmer.

The term “complementary” as used herein includes the relationship that nucleobases can form via hydrogen bonds so-called Watson-Crick base pairs (natural base pairs) or Wobble base pairs (guanine-thymine or guanine-uracil) and a relationship that a natural nucleobase and a modified nucleobase or modified nucleobases can form similar base pairs.

In the present invention, the antisense region of the nucleic acid molecule is not necessarily required to be completely complementary to at least a part of a target transcriptional product (e.g., the transcriptional product of a target gene), and it is acceptable if the base sequence has at least 70%, preferably at least 80%, and further preferably at least 90% (e.g., 95%, 96%, 97%, 98%, or 99% or more) of complementarity. The antisense region of the nucleic acid molecule can hybridize to a target transcriptional product when the base sequence is complementary (typically when the base sequence is complementary to the base sequence of at least a part of the target transcriptional product). In the double-stranded nucleic acid complex, similarly, the complementary region in the second nucleic acid strand is not necessarily required to be completely complementary to at least a part of the nucleic acid molecule that is the first nucleic acid strand, and it is acceptable if the base sequence has a complementarity of at least 70%, preferably at least 80%, and further preferably at least 90% (e.g., 95%, 96%, 97%, 98%, or 99% or more). The complementary region in the second nucleic acid strand can be annealed when the base sequence of the region is complementary to the base sequence of at least a part of the first nucleic acid strand. Base sequence complementarity can be determined by using a BLAST program or the like. Those skilled in the art can easily determine the conditions (temperature, salt concentration, and the like) under which two strands can anneal or hybridize, taking into account the degree of complementarity between the strands. Furthermore, those skilled in the art can easily design an antisense nucleic acid complementary to a target transcriptional product, e.g., based on information about the base sequence of a target gene.

Hybridization conditions may be variously stringent, e.g., low-stringent and high-stringent conditions. Low-stringent conditions may be relatively low temperature and high salt concentration conditions, e.g., 30 °C, 2×SSC, 0.1% SDS. High-stringent conditions may be relatively high temperature and low salt concentration conditions, e.g., 65 °C, 0.1×SSC, 0.1% SDS. The stringency of hybridization can be adjusted by changing conditions such as temperature and salt concentration. In this connection, 1×SSC comprises 150 mM sodium chloride and 15 mM sodium citrate.

Herein, “delivery to the central nervous system and/or the peripheral nervous system” means delivery to any site in the central nervous system and/or the peripheral nervous system or to the whole central nervous system and/or the whole peripheral nervous system. The nervous system can be divided into the central nervous system and the peripheral nervous system. The central nervous system consists of the brain and the spinal cord. The brain comprises the cerebrum (cerebral cortex, cerebral white matter, and basal ganglion), the diencephalon (thalamus and subthalamic nucleus), the cerebellum (cerebellar cortex and cerebellar nucleus), and the brainstem (midbrain, substantia nigra, pons, and medulla oblongata). The spinal cord comprises the cervical cord, the thoracic cord, the lumbar cord, the sacral cord, and the coccygeal cord. Although the “central nervous system” herein may be any of these regions, it may be particularly the cerebral cortex (frontal lobe, temporal lobe, parietal lobe, or occipital lobe), the cerebellum, the striatum, the globus pallidus, the claustrum, the hippocampus, the parahippocampal gyrus, the brainstem, the cervical cord, the thoracic cord, or the lumbar cord. The central nervous system is, e.g., a brain. The peripheral nerve consists of the cranial nerve and the spinal nerve, and comprises the ventral root of the spinal cord, dorsal root, 1st to 12th cranial nerves, cauda equina, and dorsal root ganglion.

Herein, “promoting delivery” to the central nervous system means increasing the amount of delivery to the central nervous system and/or peripheral nervous system or increasing the efficiency of delivery to the central nervous system and/or the peripheral nervous system. For example, the amount of delivery of a drug to the central nervous system and/or the peripheral nervous system under the coadministration conditions may be increased, compared with the amount of delivery of the drug under conditions where an agent for promoting delivery of the present invention is not coadministered.

A “subject” herein refers to an object to which a medicament or a pharmaceutical composition is applied. A subject comprises an individual as well as an organ, a tissue, and a cell. When the subject is an individual, any animal including a human may be applicable. For example, in addition to a human, a variety of domestic animals, domestic fowls, pets, and laboratory animals are included. Without limitation, the subject may be an individual in need of a decrease in the expression amount of a target transcriptional product, or an individual in need of treatment or prevention of a disease such as the central nervous system disease.

The term “plurality” herein refers to, e.g., 2, 2 or 3, 2 to 4, 2 to 5, 2 to 6, 2 to 7, 2 to 8, 2 to 10, 2 to 12, 2 to 14, 2 to 16, 2 to 18, 2 to 20, 2 to 25, 2 to 30, 2 to 35, 2 to 40, or more.

### (Agent for Promoting Delivery)

An agent for promoting delivery of a drug of the present aspect consists of an agent for promoting the function of Aquaporin-4 or an agent for modifying the function of Aquaporin-4.

Herein, the “agent for promoting the function of Aquaporin-4” is not limited, subject to being a substance capable of activating Aquaporin-4. Any known agent for promoting the function of Aquaporin-4 can be used. In addition, the “agent for modifying the function of Aquaporin-4” herein is not limited, subject to being a substance capable of modifying the function of Aquaporin-4. Any known agent for modifying the function of Aquaporin-4 can be used.

Examples of the agent for promoting the function of Aquaporin-4 or the agent for modifying the function of Aquaporin-4 include a compound represented by the following formula (I):

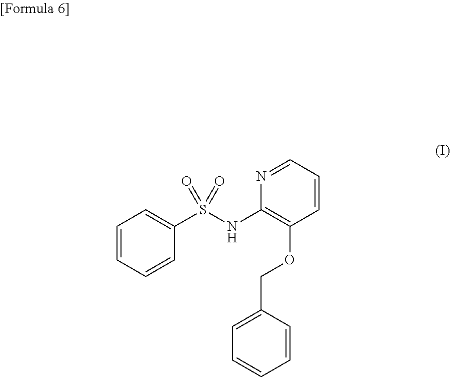 [SMILES: O=S(=O)(Nc1ncccc1OCc1ccccc1)c1ccccc1]

or a derivative thereof, or a salt of any of the compound and the salt. In this regard, WO2017/150704 and the present inventors' past literature (Huber V. J. et al., NeuroReport, 2018, 29 (9): 697-703) have disclosed that a compound represented by the above-described formula (I) can promote the function of Aquaporin-4. Such a compound is known under the common name TGN-073, which can also be referred to as N-(3-benzyloxypyridin-2-yl)-benzene-sulfonamide or 2-(phenylsulfonamido)-3-benzyloxypyridine.

A derivative of a compound represented by (I) above is any derivative capable of inducing the function-promoting activity or the function-modifying activity in Aquaporin-4. Examples of the derivative include a compound represented by the following formula (III):

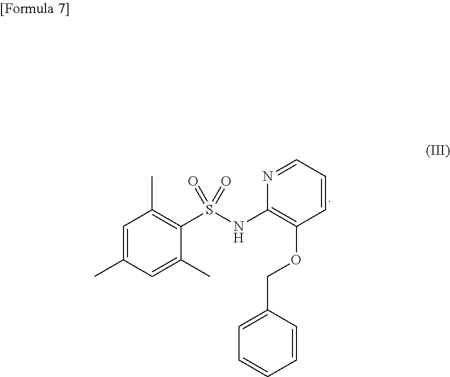 [SMILES: Cc1cc(C)c(S(=O)(=O)Nc2ncccc2OCc2ccccc2)c(C)c1]

In addition, a salt of a compound represented by the above-described formula (I) is not particularly limited, subject to being a pharmaceutically acceptable salt. Examples of the pharmaceutically acceptable salt include metal salts, salts of inorganic acids, and salts of organic acids. The metal salt may be a sodium salt, potassium salt, calcium salt, magnesium salt, or strontium salt. The salt of an inorganic acid may be a salt of hydrochloric acid, bromic acid, phosphoric acid, sulfuric acid, or disulfuric acid. The salt of an organic acid may be a salt of formic acid, acetic acid, propionic acid, lactic acid, oxalic acid, tartaric acid, malic acid, maleic acid, citric acid, fumaric acid, besylic acid, camsylic acid, edisylic acid, trichloroacetic acid, trifluoroacetic acid, benzoic acid, gluconic acid, methanesulfonic acid, glycolic acid, succinic acid, 4-toluenesulfonic acid, galacturonic acid, embonic acid, glutamic acid, ethanesulfonic acid, benzenesulfonic acid, p-toluenesulfonic acid, or asparagic acid.

In one embodiment, a compound represented by the formula (I), or a derivative thereof, is bound to a carrier molecule. The carrier molecule is a molecule bound to an agent for promoting delivery, for the purpose of regulating the efficiency with which an agent for promoting delivery is delivered to a delivery site of interest. The carrier molecule enables the efficiency of delivery to a delivery site of interest to be regulated on the basis of controlling the molecular size, hydrophilicity, hydrophobicity, and the like of the carrier molecule. Examples of the carrier molecule include, but are not particularly limited to, proteins (e.g., 1 kDa or more proteins), polymers, lipid molecules, and contrast agents. Examples of the protein include albumin, lipoproteins (e.g., HDL like particles), antibodies, and antibody fragments (e.g., IgG, Fc, and modified Fc). Examples of the polymer include polyethylene glycol (PEG) and PEG grafted polymers. Examples of the lipid include tocopherol, cholesterol, fatty acids, phospholipid, and the below-described lipids.

In a further embodiment, a compound represented by the formula (I) or a derivative thereof can be bound to or associated with a microbubble, micelle, or liposome. The mode of binding or association is not limited, and, e.g., can be via the above-described carrier molecule, such as a lipid or a polymer, in which the carrier molecule is bound to a compound represented by the formula (I) or a derivative thereof.

Controlling the molecular size, hydrophilicity, and hydrophobicity of an agent for promoting delivery of the present invention makes it possible to prevent the agent from permeating into a tissue at or around a site of administration, and thereby prevent a decrease in the efficiency of delivery to a delivery site of interest, and thus, is suitable.

### (Drug)

Herein, a “drug” (also referred to as a medicament, a medicine, or a pharmaceutical) is not particularly limited, and encompasses a nucleic acid medicine, peptide, small molecule compound, viral vector, cellular medicine, nanoparticle, liposome, micelle, or exosome. In this regard, a drug to be used in combination with an agent for promoting delivery of the present invention herein, and be delivered to the central nervous system and/or the peripheral nervous system is often referred to as a “drug to be delivered to a nervous system”.

A “peptide” herein refers to an amino acid polymer having one or more peptide bonds. The peptide is not limited by the number of amino acid residues comprised in the peptide.

Accordingly, the “peptide” encompasses an oligopeptide comprising several amino acid residues, such as a dipeptide or a tripeptide, and a polypeptide comprising many amino acid residues. Accordingly, the peptide encompasses not only what is called a protein but also a fragmented peptide and a peptide linked with another peptide via a peptide bond. Examples of the peptide include antibodies or antibody fragments and enzymes. In this regard, to the peptide, an exogenous peptide, such as a protein medicine (e.g., a protein formulation), corresponds in principle, and the peptide does not comprise a degradation product of proteins, other waste products in cells, or the like. In addition, the peptide may be naturally occurring or non-natural. Furthermore, the peptide may be cyclic or non-cyclic.

In one embodiment, the peptide is an antibody or an antibody fragment.

Herein, an “antibody” means a protein that exhibits immunoresponsiveness to an antigen. The biological species of the origin of the antibody is not particularly limited. The antibody is preferably derived from a bird or a mammal. Examples include chickens, ostriches, mice, rats, guinea pigs, rabbits, goats, donkeys, sheep, camels, horses, and humans. The antibody may be a full-length antibody.

Herein, a “fragment” of an antibody is an antibody fragment that consists of part of an antibody, and exhibits immunoresponsiveness to an antigen in the same manner as an antibody. The antibody fragment is also an antigen-binding fragment. Examples include: Fab; Fab′; F(ab′)₂; Fv fragment; Fv fragment stabilized by a disulfide bond (dsFv); (dsFv)₂; bispecific dsFv (dsFv-dsFv′); diabody stabilized by a disulfide bond (ds diabody); single-chain antibody molecule (scFv); dimeric scFv (divalent diabody); multispecific antibody; heavy-chain antibody, such as a camelized single-domain antibody (camelized antibody; VHH antibody); nanobody; domain antibody; and divalent domain antibody. Fab is an antibody fragment generated by allowing an IgG molecule to be cleaved on the N-end side of a disulfide bond by papain of the hinge region. An H-chain constant region (heavy-chain constant region; hereinafter referred to as C_{H}) is constituted by three domains (C_{H}1, C_{H}2, and C_{H}3), among which C_{H}1 is adjacent to V_{H}. Fab is composed of C_{H}1, V_{H}, and a full-length L chain. Fab′ has a slightly longer H chain than Fab by a portion comprising a hinge region, but has substantially the same structure as Fab. Fab′ can be obtained by reducing an Fab′ dimer (F (ab′)₂) under mild conditions, and cleaving the disulfide bond of the hinge region, in which the Fab′ dimer is generated by allowing an IgG molecule to be cleaved on the C-end side of the disulfide bond of the hinge region by pepsin. Any of these antibody fragments encompasses an antigen-binding site, and thus, has the capability to bind specifically to an antigen epitope.

Herein, a “small molecule compound” is not particularly limited, and may be a therapeutic drug, psychiatric drug, or anticancer agent for the central nervous system disease or the peripheral nervous system disease. In this regard, herein, the above-described agent for promoting delivery should itself be excluded from the small molecule compound to be delivered to a nervous system.

Herein, the “viral vector” is not particularly limited. For example, viral vectors that can be used are a retroviral vector (comprising an oncoretroviral vector, lentiviral vector, and pseudotyped vector), adenoviral vector, adeno-associated viral (AAV) vector, simian virus vector, vacciniaviral vector, Sendaiviral vector, Epstein-Barr viral (EBV) vector, and HSV vector. In order not to self-replicate in an infected cell, a viral vector lacking in replication competence may be used. A viral vector can encode a gene for gene therapy.

Herein, a “cellular medicine” is not particularly limited. For example, a cell to be used to treat any central nervous system disease or peripheral nervous system disease can be used. Examples of a cell comprised in the cellular medicine include nerve cells, T-cells (e.g., CAR-T-cells), NK cells, NKT cells, hematopoietic stem cells, peripheral blood mononuclear cells, mesenchymal stem cells, iPS cells, and ES cells.

Herein, a “nanoparticle” refers to a particle having a particle size of a nanometric (nm) order. A nanoparticle means a particle having a particle size of 1 nm to hundreds of nm, in principle. Specific examples of the nanoparticle include polymer nanoparticles, metal nanoparticles, and dendrimers.

Herein, a “liposome” means a vesicle comprising a lipid membrane and an aqueous medium encapsulated in the lipid membrane. The lipid membrane of a liposome is composed of one or two or more lipid layers. For example, a liposome is composed of a lipid bilayer comprising a phospholipid and the like.

Herein, a “micelle” means a vesicle formed by one layer of a molecular film. Examples of a constituent of a micelle include amphiphilic molecules such as a surfactant.

The above-described nanoparticle, liposome, and micelle can comprise a nucleic acid medicine, peptide, small molecule compound, or the like, and is useful for delivery of a medicament.

Herein, an “exosome” is a vesicle that is secreted from a cell, and encapsulated in a lipid bilayer membrane. An exosome is derived from a multivesicular endosome, and in some cases, comprises a nucleic acid, such as RNA or DNA, or a biological substance, such as protein, in it when released into an extracellular environment.

### (Nucleic Acid Medicine)

Herein, a “nucleic acid medicine” is a drug comprising any nucleic acid molecule. Herein, the nucleic acid medicine means a nucleic acid medicine comprising two or more nucleosides, in principle. A nucleoside comprised in the nucleic acid medicine may be a natural nucleoside or a non-natural nucleoside. In addition, a nucleic acid molecule comprised in the nucleic acid medicine may be a single-stranded, double-stranded, or more multistranded nucleic acid. Specific examples of the nucleic acid medicine include, but are not limited to: an antisense nucleic acid described below as a nucleic acid molecule; a heteronucleic acid described below as a double-stranded nucleic acid complex; siRNA; shRNA; miRNA; mRNA; lncRNA; an aptamer; plasmid DNA; decoy; bait nucleic acid; ribozyme; and nucleic acid vector such as a plasmid vector.

### (Nucleic Acid Molecule)

In one embodiment, the nucleic acid medicine comprises a nucleic acid molecule which is capable of hybridizing to at least part of a target gene or a transcriptional product thereof, and has an antisense effect for the target gene or the transcriptional product thereof.

The base length of the nucleic acid molecule is not particularly limited, and may be at least 8 bases in length, at least 9 bases in length, at least 10 bases in length, at least 11 bases in length, at least 12 bases in length, at least 13 bases in length, at least 14 bases in length, or at least 15 bases in length. Furthermore, the base length of the nucleic acid molecule may be 40 bases in length or less, 35 bases in length or less, 30 bases in length or less, 25 bases in length or less, 24 bases in length or less, 23 bases in length or less, 22 bases in length or less, 21 bases in length or less, 20 bases in length or less, 19 bases in length or less, 18 bases in length or less, 17 bases in length or less, or 16 bases in length or less. The base length of the nucleic acid molecule may be, e.g., 10 to 40 bases in length, 12 to 30 bases in length, or 15 to 25 bases in length. The length can be determined according to the balance between the strength of the antisense effect and the specificity of the nucleic acid strand to the target, among other factors, such as cost and synthesis yield. When a nucleic acid such as an aptamer is bound to a nucleic acid molecule, the base length of the nucleic acid molecule as a whole may be the sum of the above-described base length and the base length of the bound nucleic acid.

A nucleoside comprised in the nucleic acid molecule may be a natural nucleoside (deoxyribonucleoside, ribonucleoside, or both thereof) and/or a non-natural nucleoside.

In one embodiment, the nucleic acid molecule may be a mixmer.

In another embodiment, the nucleic acid molecule may be a gapmer.

The central region (gap region) of the gapmer may be, e.g., 3 to 12 bases in length, 4 to 11 bases in length, 5 to 10 bases in length, 6 to 9 bases in length, or 7 or 8 bases in length.

In addition, the base length of each of the 5′-wing region and the 3′-wing region of the gapmer may be independently at least 2 bases in length, e.g., 2 to 10 bases in length, 2 to 7 bases in length, 3 to 5 bases in length, 3 or 4 bases in length, or 3 bases in length.

The nucleic acid molecule can comprise a 2′-modified nucleoside and/or bridged nucleoside in the 5′-wing region and the 3′-wing region. The 2′-modified nucleoside may be, e.g., 2′-O-methyl-modified nucleoside, 2′-O-methoxyethyl-modified nucleoside, 2′-O-[2-(N-methylcarbamoyl)ethyl]-modified nucleoside, or 2′-fluoro-modified nucleoside. In addition, the bridged nucleoside may be, e.g., LNA nucleoside, 2′,4′-BNA^{NC} nucleoside, cEt BNA nucleoside, ENA nucleoside, AmNA nucleoside, GuNA nucleoside, scpBNA nucleoside, scpBNA2 nucleoside, or BANA3 nucleoside. Preferably, 2′-O-methyl-modified nucleoside, 2′-O-methoxyethyl-modified nucleoside, 2′-LNA, and ENA may be combined. The kind of modification may include one to four, two or three, e.g., two, which may be the same or different from each other in the 5′-wing region and the 3′-wing region.

In one embodiment, the nucleic acid molecule comprises a 2′-modified nucleoside at the terminal base position adjacent to the central region in the 5′-wing region, the 2nd base position from 5′ side of the central region, and/or the 8th base position from 5′ side of the central region. The “the terminal base position adjacent to the central region in the 5′-wing region” means a base position located at the most 3′ end in the 5′-wing region. In addition, herein, the “Nth base position from the 5′ side of the central region” means a base located at the Nth position in the 5′ side to 3′ side direction starting from a terminal base adjacent to the 5′-wing region in the central region. Therefore, the 2nd base position from the 5′ side of the central region means the base located at the 2nd position counting in the 3′ direction starting from the terminal base adjacent to the 5′-wing region in the central region, and the 8th base position from the 5′ side of the central region means the base located at the 8th position counting in the 3′ direction starting from the terminal base adjacent to the 5′-wing region in the central region. The 2′-modification in the 2′-modified nucleoside is not limited, and may be, e.g., a 2′-O-methyl group, a 2′-O-[2-(N-methylcarbamoyl)ethyl] group, a 2′-O-methoxyethyl group, or a 2′-fluoro group, preferably a 2′-O-methyl group.

In a further embodiment, the 5′-wing region and/or the 3′-wing region consist of two or more 2′-modified nucleosides and/or bridged nucleosides linked via an internucleoside linkage.

In the gapmer, examples of the base length of each region of the 5′-wing region, the central region, and the 3′-wing region include 2-12-3, 3-12-2, 3-12-3, 4-12-3, 2-11-3, 3-11-2, 3-11-3, 4-11-3, 2-10-3, 3-10-2, 3-10-3, 4-10-3, 2-9-3, 3-9-2, 3-9-3, 4-9-3, 2-8-3, 3-8-2, 3-7-3, 4-6-3, 3-6-4, 4-5-4, 4-7-3, 3-7-4, 4-6-4, 5-6-3, 3-6-5, 3-7-5, 5-7-3, 4-7-4, 4-6-5, 5-6-4, 5-5-5, and 5-6-5. Here, in the notation “A-B-C,” “A” denotes the base length of the 5′-wing region, “B” denotes the base length of the central region, and “C” denotes the base length of the 3′-wing region.

The internucleoside linkage in the nucleic acid molecule may be a naturally occurring internucleoside linkage and/or a modified internucleoside linkage. Without limitation, at least one, at least two, or at least three internucleoside linkages from an end (5′ end, 3′ end, or both the ends) of the nucleic acid molecule are preferably modified internucleoside linkages. In this regard, e.g., two internucleoside linkages from the end of a nucleic acid strand refers to an internucleoside linkage closest to the end of the nucleic acid strand, and an internucleoside linkage positioned next thereto on the opposite side to the end of the nucleic acid strand. Modified internucleoside linkages in the terminal region of a nucleic acid strand are preferred because they can reduce or inhibit undesired degradation of the nucleic acid strand.

In one embodiment, all or a part of the internucleoside linkages in the nucleic acid molecule may be modified internucleoside linkages. The modified internucleoside linkage may be a phosphorothioate linkage.

The nucleic acid molecule may comprise, as a whole or in part, a nucleoside mimic or a nucleotide mimic. The nucleotide mimic may be a peptide nucleic acid and/or a morpholino nucleic acid. In a further embodiment, 25% or more, 33% or more, 40% or more, 50% or more, 60% or more, 70% or more, 80% or more, 90% or more, 95% or more, or 100% (all) of the nucleic acids in the nucleic acid molecule are morpholino nucleic acids. The internucleoside linkage between morpholino nucleic acids is not limited. Part or all of the internucleoside linkages may be a phosphorodiamidate linkage.

The antisense effect on the target transcriptional product of the nucleic acid molecule can be measured by a method publicly known in the art. For example, after introducing a nucleic acid molecule into a cell and the like, it can be measured using a publicly known technique such as Northern blotting, quantitative PCR, or Western blotting. By measuring the expression amount of a target gene or the level of a target transcriptional product in specific tissues (e.g., the amount of RNA such as the amount of mRNA, and the amount of cDNA), it can be judged whether or not the target gene expression is suppressed by the nucleic acid molecule in these sites. When the expression amount measured of the target gene or the level measured of the target transcriptional product is decreased by at least 20%, at least 25%, at least 30%, at least 40%, or at least 50%, compared with a negative control (e.g., vehicle administration or no treatment), the measurement shows that the subject nucleic acid compound can produce an antisense effect.

### (Double-Stranded Nucleic Acid Complex)

In one embodiment, the nucleic acid medicine consists of a double-stranded nucleic acid complex comprising a first nucleic acid strand consisting of any of the above-described nucleic acid molecules and a second nucleic acid strand comprising a base sequence complementary to the first nucleic acid strand.

The double-stranded nucleic acid complex comprises the first nucleic acid strand and the second nucleic acid strand. The first nucleic acid strand is any of the above-described nucleic acid molecules, and thus, the description thereof is omitted here.

In the double-stranded nucleic acid complex, the second nucleic acid strand is a nucleic acid molecule comprising a base sequence complementary to the first nucleic acid strand. In the double-stranded nucleic acid complex, the second nucleic acid strand is annealed to the first nucleic acid strand via a hydrogen bond of a complementary base pair.

In the double-stranded nucleic acid complex, the second nucleic acid strand can comprise a deoxyribonucleoside, a 2′-modified nucleoside, a 5′-modified nucleoside, and/or a bridged nucleoside. For example, in the second nucleic acid strand, all of the nucleosides in a region consisting of a base sequence complementary to the central region of the first nucleic acid strand may be (a) deoxyribonucleosides; (b) deoxyribonucleosides and ribonucleosides; (c) deoxyribonucleosides and 2′-modified nucleosides; (d) ribonucleosides and 2′-modified nucleosides; or (e) deoxyribonucleosides, ribonucleosides, and 2′-modified nucleosides.

In one embodiment, the second nucleic acid strand comprises a region comprising at least three or at least four consecutive ribonucleosides and/or deoxyribonucleosides which are complementary to at least three or at least four consecutive deoxyribonucleosides in the central region of the first nucleic acid strand.

In a further embodiment, the second nucleic acid strand may comprise a region consisting of a base sequence complementary to the 5′-wing region and/or the 3′-wing region in the first nucleic acid strand. In the second nucleic acid strand, the region consisting of a base sequence complementary to the 5′-wing region and/or the 3′-wing region in the first nucleic acid strand may comprise at least one non-natural nucleoside, wherein the non-natural nucleoside may a bridged nucleoside and/or a 2′-modified nucleoside. In addition, the 2′-modified group of the 2′-modified nucleoside in the second nucleic acid strand may be a 2′-O-methyl group or a 2′-O-methoxyethyl group. When the first nucleic acid strand and the second nucleic acid strand both comprise a bridged nucleoside and/or a 2′-modified nucleoside, the bridged nucleoside and/or the 2′-modified nucleoside in the first nucleic acid strand and the second nucleic acid strand may be the same or different from each other.

The internucleoside linkage in the second nucleic acid strand may be a naturally occurring internucleoside linkage and/or a modified internucleoside linkage. Without limitation, at least one, at least two, or at least three internucleoside linkages from an end (5′ end, 3′ end, or both the ends) of the second nucleic acid strand are preferably modified internucleoside linkages. In one embodiment, all or a part of the internucleoside linkages in the second nucleic acid strand may be modified internucleoside linkages. In one embodiment, the second nucleic acid strand may comprise a modified internucleoside linkage in a region consisting of a base sequence complementary to the 5′-wing region and/or the 3′-wing region in the first nucleic acid strand. The modified internucleoside linkage may be a phosphorothioate linkage.

In a further embodiment, the second nucleic acid strand may comprise a 2′-modified nucleoside (e.g., 2′-O-methyl-modified nucleoside, 2′-O-methoxyethyl-modified nucleoside, 2′-O-[2-(N-methylcarbamoyl)ethyl]-modified nucleoside, or 2′-fluoro-modified nucleoside). In the second nucleic acid strand, the number of 2′-modified nucleosides is not limited. For example, at least 20%, at least 30%, at least 40%, at least 50%, at least 60%, at least 70%, at least 75%, at least 80%, at least 85%, at least 90%, at least 95%, or 100% of the total number of nucleosides in the second nucleic acid strand may be 2′-modified nucleosides. In one embodiment, all of the nucleosides in the second nucleic acid strand are 2′-modified nucleosides.

In a further embodiment, the second nucleic acid strand may comprise one or two or more consecutive 2′-modified nucleosides (e.g., 2′-O-methyl-modified nucleoside, 2′-O-methoxyethyl-modified nucleoside, 2′-O-[2-(N-methylcarbamoyl)ethyl]-modified nucleoside, or 2′-fluoro-modified nucleoside) positioned at the 5′ end and/or one or two or more consecutive 2′-modified nucleosides positioned at the 3′ end. The number of the 2′-modified nucleosides positioned at the 5′ end and/or the 3′ end is not limited. For example, the second nucleic acid strand may comprise one or two, three, four, five, six, or seven consecutive 2′-modified nucleosides at the 5′ end, and/or one or two, three, four, five, six, or seven consecutive 2′-modified nucleosides at the 3′ end.

In one embodiment, the first nucleic acid strand and/or the second nucleic acid strand may comprise a modified nucleobase. The number of modified nucleobases is not limited, and may be, for example, at least one, at least two, at least three, at least four, at least five, or at least six

In one embodiment, the second nucleic acid strand may comprise a noncomplementary base, and/or one or more insertion sequences and/or deletions, relative to the first nucleic acid strand. The number of noncomplementary bases in the second nucleic acid strand is not limited, and may be, e.g., 1 to 10, 1 to 9, 1 to 8, 1 to 7, 1 to 6, 1 to 5, 1 to 4, 1 to 3, 1 to 2, or 1 or 2. The base length of the insertion sequence in the second nucleic acid strand is not limited, and may be, e.g., 1 to 10, 1 to 9, 1 to 8, 1 to 7, 1 to 6, 1 to 5, 1 to 4, 1 to 3, 1 to 2, or 1 or 2. The base length of consecutive deletions in the second nucleic acid strand is not limited, and may be, e.g., 1 to 10, 1 to 9, 1 to 8, 1 to 7, 1 to 6, 1 to 5, 1 to 4, 1 to 3, 1 to 2, or 1 or 2. A region composed of a noncomplementary base or an insertion sequence may form a bulge.

In one embodiment, the second nucleic acid strand may further comprise at least one overhang region positioned on one or both of the 5′ end side and the 3′ end side of the complementary region. An example of this embodiment is described in WO2018/062510. The term “overhang region” refers to a region adjacent to a complementary region, namely a nucleotide region in the second nucleic acid strand in which the 5′ end of the second nucleic acid strand extends beyond the 3′ end of the first nucleic acid strand and/or the 3′ end of the second nucleic acid strand extends beyond the 5′ end of the first nucleic acid strand when the first nucleic acid strand and the second nucleic acid strand are annealed to form a double-stranded structure, or protruding from the double-stranded structure. The overhang region in the second nucleic acid strand may be located at the 5′ end side of the complementary region or at the 3′ end side. The overhang region in the second nucleic acid strand may be located at the 5′ end side and 3′ end side of the complementary region. The base length of the overhang region is not particularly limited, and may be 1 to 30 bases in length or 1 to 20 bases in length.

In one embodiment, a functional moiety may be bound to the first nucleic acid strand and/or the second nucleic acid strand, e.g., the second nucleic acid strand. The linkage between the first nucleic acid strand and/or second nucleic acid strand and the functional moiety may be a direct linkage or an indirect linkage via another substance, and in some embodiments, it is preferable that the first nucleic acid strand and/or second nucleic acid strand and the functional moiety are directly bound by covalent bonding, ionic bonding, hydrogen bonding, or the like, and from the viewpoint of obtaining a more stable bond, covalent bonding is more preferable.

In one embodiment, the structure of the “functional moiety” is not particularly limited, and provides a desired function to the double-stranded nucleic acid complex to which the functional moiety is bound. The desired function includes a labeling function, a purification function, and a delivery function to a target. A moiety that provides a labeling function is, e.g., a lipid or a peptide. Specific examples of the moiety include compounds such as a fluorescent protein and luciferase. Examples of a moiety that provides a purification function include compounds such as biotin, avidin, His-tag peptide, GST-tag peptide, and FLAG-tag peptide. In addition, a molecule having an activity for delivery of a double-stranded nucleic acid complex in an embodiment to a target site is preferably bound as a functional moiety to the first nucleic acid strand and/or the second nucleic acid strand, from the viewpoint that the first nucleic acid strand is efficiently delivered to a target site at a high specificity and expression of a target gene is very effectively suppressed by the nucleic acid. Examples of a moiety for providing a delivery function to a target include a lipid, an antibody, an aptamer, and a ligand to a particular receptor.

In one embodiment, the first nucleic acid strand and/or the second nucleic acid strand, e.g., the second nucleic acid strand, is bound to a lipid. Examples of the lipid include, but are not limited to, tocopherol, cholesterol, fatty acid, phospholipid (e.g., phosphatidylethanolamine) and analogs thereof; folic acid, vitamin C, vitamin B1, and vitamin B2; estradiol, androstane, and analogs thereof; steroid and an analog thereof; ligand of LDLR, SRBI or LRP1/2; FK-506, and cyclosporine; and lipids described in WO2019/182109 and WO2019/177061. The lipid may be a tocopherol or an analog thereof and/or a cholesterol or an analog thereof, a substituted or unsubstituted C₁₋₃₀ alkyl group, a substituted or unsubstituted C₂₋₃₀ alkenyl group, or a substituted or unsubstituted C₁₋₃₀ alkoxy group. In one embodiment, the second nucleic acid strand may be bound to tocopherol or cholesterol, or an analog thereof.

“Tocopherol” is herein a methylated derivative of tocorol which is a liposoluble vitamin (vitamin E) having a cyclic structure called chroman. Tocorol has a strong antioxidant effect, and therefore functions in vivo as an antioxidant substance to scavenge free radicals produced by metabolism and protect cells from damage.

A plurality of different types of tocopherol consisting of α-tocopherol, β-tocopherol, γ-tocopherol, and δ-tocopherol are known based on the position of the methyl group bound to chroman. A tocopherol herein may be any types of tocopherol. In addition, examples of the analog of tocopherol include various unsaturated analogs of tocopherol, such as α-tocotrienol, β-tocotrienol, γ-tocotrienol, and δ-tocotrienol. Preferably, tocopherol is α-tocopherol.

“Cholesterol” is herein a kind of sterol, also called steroid alcohol, which is especially abundant in animals. Cholesterol exerts an important function in the metabolic process in vivo, and in animal cells, it is also a major constituent of the membrane system of a cell, together with phospholipid. In addition, the cholesterol analog refers to various cholesterol metabolism products and their analogs, which are alcohols having a sterol backbone. Examples thereof include, but are not limited to, cholestanol, lanosterol, cerebrosterol, dehydrocholesterol, and coprostanol.

Herein, an “analog” or a “derivative” refers to a compound having a similar structure and property having the same or a similar basic backbone. The analog comprises, e.g., a biosynthetic intermediate, a metabolism product, and a compound having a substituent. Those skilled in the art can determine whether or not a compound is an analog of another compound based on common general technical knowledge.

The functional moiety may be linked to the 5′ end, the 3′ end, or both ends of the first nucleic acid strand and/or the second nucleic acid strand. Alternatively, the functional moiety may be linked to a nucleotide at an interior position in the first nucleic acid strand and/or the second nucleic acid strand. The first nucleic acid strand and/or the second nucleic acid strand comprise two or more functional moieties, such as lipids, which may be linked to a plurality of positions in the first nucleic acid strand and/or the second nucleic acid strand, and/or may be linked, in the form of one group, to one position in the first nucleic acid strand and/or the second nucleic acid strand. One functional moiety may be linked to each of the 5′ end and 3′ end of the first nucleic acid strand and/or the second nucleic acid strand.

The linkage between the first nucleic acid strand and/or the second nucleic acid strand and the functional moiety may be a direct linkage or an indirect linkage via another substance. In a specific embodiment, however, it is preferable that the functional moiety is directly bound to the first nucleic acid strand and/or the second nucleic acid strand via a covalent bond, an ionic bond, a hydrogen bond, or the like, and a covalent bond is more preferable considering that a more stable bond can be obtained.

The functional moiety may also be bound to the first nucleic acid strand and/or the second nucleic acid strand via a cleavable or uncleavable linker. In this case, the first nucleic acid strand and the second nucleic acid strand can be linked via a linker to form a single strand. However, even in that case, the functional region has the same configuration as the double-stranded nucleic acid complex, and therefore such a single-stranded nucleic acid is herein also encompassed as an embodiment of the double-stranded nucleic acid complex in the present invention. The linker can be any polymer. Examples thereof include a polynucleotide, polypeptide, alkylene. Specifically, it can be composed of a natural nucleotide such as DNA or RNA, or a non-natural nucleotide such as a peptide nucleic acid or a morpholino nucleic acid. When a linker consists of a nucleic acid, the chain length of a linker may be at least one base, or, e.g., a chain length of from 3 to 10 bases or from 4 to 6 bases. It is preferably 4 bases in length. In this case, the linker can take the form of a hinge (hairpin loop). The position of the linker can be either on the 5′ side or the 3′ side of the first nucleic acid strand. For example, in the case of a configuration in which the second nucleic acid strand is bound to the 5′ side of the first nucleic acid strand, the 5′ end of the first nucleic acid strand and the 3′ end of the second nucleic acid strand are linked via a linker.

A “cleavable linker” refers to a linking group that is cleaved under physiological conditions, e.g., in a cell or in an animal body (e.g., in a human body). In a specific embodiment, a cleavable linker is selectively cleaved by an endogenous enzyme such as a nuclease. Examples of the cleavable linker include an amide, an ester, one or both esters of a phosphodiester, a phosphoester, a carbamate, and a disulfide bond, as well as a natural DNA linker.

An “uncleavable linker” refers to a linker that is not cleaved under physiological conditions, e.g., in a cell or in an animal body (e.g., in a human body). Examples of an uncleavable linker include, but are not limited to, a phosphorothioate linkage, modified or unmodified deoxyribonucleosides linked by a phosphorothioate linkage, and a linker consisting of modified or unmodified ribonucleosides. There is no particular restriction on the chain length, when a linker is a nucleic acid such as DNA, or an oligonucleotide, however, it may usually be from 2 to 20 bases in length, from 3 to 10 bases in length, or from 4 to 6 bases in length.

Specific examples of the linker include a linker represented by the following Formula (IV).

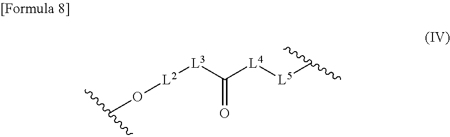

(wherein
L² represents a substituted or unsubstituted C₁-C₁₂ alkylene group (e.g., propylene, hexylene, or dodecylene), substituted or unsubstituted C₃-C₈ cycloalkylene group (e.g., cyclohexylene), —(CH₂)₂—O—(CH₂)₂—O—(CH₂)₂—O—(CH₂)₃—, —(CH₂)₂—O—(CH₂)₂—O—(CH₂)₂—O—(CH₂)₂—O—(CH₂)₃—, or CH(CH₂—OH)—CH₂—O—(CH₂)₂—O—(CH₂)₂—O—(CH₂)₂—O—(CH₂)₃—; L³ represents —NH— or a bond; L⁴ represents a substituted or unsubstituted C₁-C₁₂ alkylene group (e.g., ethylene, pentylene, heptylene, or undecylene), substituted or unsubstituted C₃-C₈ cycloalkylene group (e.g., cyclohexylene), —(CH₂)₂—[O—(CH₂)₂]ₘ—, or a bond, wherein m represents an integer of 1 to 25; and L⁵ represents —NH—(C═O)—, —(C—O)—, or a bond (here, the substitution is preferably made with a halogen atom).

In one embodiment, in a linker represented by the formula (VI), L² is an unsubstituted C₃-C₆ alkylene group (e.g., propylene or hexylene), —(CH₂)₂—O—(CH₂)₂—O—(CH₂)₂—O—(CH₂)₃—, or —(CH₂)₂—O—(CH₂)₂—O—(CH₂)₂—O—(CH₂)₂—O—(CH₂)₃—; L³ is-NH—; and L⁴ and L⁵ are each a bond.

In one embodiment, the linker comprises a nucleic acid, polyether group, and/or alkylamino group. The nucleic acid may consist of, e.g., 1 or 2 to 10 nucleosides and/or non-natural nucleosides linked via internucleoside linkages. Examples of the polyether group include a polyethylene glycol group, triethylene glycol group, or tetraethylene glycol group. Examples of the alkylamino group include a hexylamino group.

The base length of the first nucleic acid strand and the second nucleic acid strand is not particularly limited, and may be at least 8 bases in length, at least 9 bases in length, at least 10 bases in length, at least 11 bases in length, at least 12 bases in length, at least 13 bases in length, at least 14 bases in length, or at least 15 bases in length. Furthermore, the base length of the first nucleic acid strand and the second nucleic acid strand may be 40 bases in length or less, 35 bases in length or less, 30 bases in length or less, 25 bases in length or less, 24 bases in length or less, 23 bases in length or less, 22 bases in length or less, 21 bases in length or less, 20 bases in length or less, 19 bases in length or less, 18 bases in length or less, 17 bases in length or less, or 16 bases in length or less. The first nucleic acid strand and the second nucleic acid strand may have identical lengths or different lengths (e.g., one of them is shorter or longer by 1 to 3 bases). The double-stranded structure formed by the first nucleic acid strand and the second nucleic acid strand may comprise a bulge. The length can be determined according to the balance between the strength of the antisense effect and the specificity of the nucleic acid strand to the target, among other factors, such as cost and synthesis yield. When a nucleic acid such as an aptamer is bound to a first nucleic acid strand and/or a second nucleic acid strand, the base length of the first nucleic acid strand and/or the second nucleic acid strand as a whole may be the sum of the above-described base length and the base length of the bound nucleic acid. In this case, the base length of the nucleic acid to be bound is not limited, and may be, e.g., at least 10 bases in length, at least 15 bases in length, or at least 20 bases in length, or may be 100 bases in length or less, 80 bases in length or less, 60 bases in length or less, 40 bases in length or less, or 30 bases in length or less.

(siRNA)

In one embodiment, the nucleic acid medicine is siRNA. Herein, “siRNA” (short-interfering RNA) means a double-stranded nucleic acid having approximately 19 to 25 base pairs, and capable of inducing reduction of the expression of a target gene, through RNAi. siRNA is composed of two nucleic acid strands: a guide strand and a passenger strand that are described below. In the present invention, two nucleic acid strands constituting siRNA may comprise not only a ribonucleoside but also a deoxynucleoside and/or any modified nucleoside. siRNA capable of inducing the RNAi activity is not limited to an siRNA consisting of a ribonucleoside, and an siRNA comprising a deoxynucleoside or a modified nucleoside is also taken in the below-described RISC, and able to recognize a target mRNA.

Herein, “RNAi” (RNA interference) refers to a phenomenon in which the expression of a target gene is specifically suppressed in a cell into which a double-stranded nucleic acid strand, such as siRNA comprising a sequence complementary to a target gene sequence, has been introduced therein. RNAi based on siRNA can be explained as follows. First, one of the strands of siRNA introduced into a cell is taken into a complex called RISC (RNA-induced Silencing Complex), and recognizes mRNA of a target gene having a highly complementary sequence. The mRNA of the target gene is cleaved by RISC at the central portion of the highly complementary sequence. Then, the mRNA cleaved can be degraded.

Specifically, siRNA in the present invention is composed of: a guide strand capable of hybridizing with at least part of a target gene or a transcriptional product thereof; and a passenger strand comprising a base sequence complementary to the guide strand.

Herein, a “guide strand” means a nucleic acid strand comprising a sequence complementary to mRNA of a target gene. In addition, a “passenger strand” means a nucleic acid strand comprising a sequence complementary to the guide strand (i.e., comprising a sequence homologous to mRNA of the target gene). The guide strand is annealed with the passenger strand to generate siRNA. The guide strand can bind to mRNA of the target gene to induce RNAi.

In one embodiment, siRNA may comprise any one or more selected from the group consisting of a deoxyribonucleoside, 2′-modified nucleoside, 5′-modified nucleoside, and bridged nucleoside. Such a modified nucleoside may be positioned in the guide strand and/or passenger strand of siRNA.

In addition, siRNA may encompass any of the various configurations described above in reference to the double-stranded nucleic acid complex. That is, the guide strand of siRNA in the present invention can encompass each of the configurations described in reference to the above-described first nucleic acid strand, and in the same manner, the passenger strand can encompass each of the configurations described in reference to the second above-described nucleic acid strand. For example, siRNA in the present invention may be bound to a functional moiety. The functional moiety may be a lipid or a peptide in the same manner as in the case of a double-stranded nucleic acid complex. In addition, the lipid may be cholesterol or an analog thereof, tocopherol or an analog thereof, folic acid, phosphatidylethanolamine, or a substituted or unsubstituted C₁₆₋₃₀ alkyl group.

In one embodiment, a nucleic acid medicine in the present invention may comprise two siRNAs (hereinafter, the siRNAs are referred to as a first siRNA and a second siRNA respectively). For example, the guide strands comprised in the two siRNAs (the first guide strand comprised in the first siRNA and the second guide strand comprised in the second siRNA) may be bound to each other via a linker. In the first guide strand and the second guide strand, the binding position of the linker is not particularly limited, and may be the 5′ end and/or the 3′ end respectively. Preferably, the 3′ end of the first guide strand and the 3′ end of the second guide strand are bound to the linker. Examples of the linker are in accordance with the examples described in reference to the double-stranded nucleic acid complex, and, e.g., comprise a nucleic acid, polyether group, and/or alkylamino group. In this regard, a structure in which two siRNAs are linked with each other is known as a divalent siRNA (Alterman J. F. et al., Nature Biotechnology, 2019, 37:884-894).

### (Dosing Form of Agent for Promoting Delivery/Drug)

Using an agent for promoting delivery of the present invention in combination with a drug to be delivered to a nervous system makes it possible to increase the efficiency of delivery of the drug to the central nervous system. The administration route and/or the administration time may be the same or different between the drug and an agent for promoting delivery of the present invention.

The dosing form of an agent for promoting delivery of the present aspect and a drug to be delivered to a nervous system is not particularly limited. The administration may be systemic administration or topical administration. The administration route may be oral or parenteral administration. Specific examples of the parenteral administration include intrathecal administration (intraventricular administration, suboccipital puncture, or lumbar puncture), transnasal administration, intravenous administration, intraarterial administration, administration by blood transfusion, intraperitoneal administration, intraocular administration, intramuscular administration, subcutaneous administration (comprising implantable continuous subcutaneous administration), intradermal administration, intravesical administration, intravaginal administration, rectal administration, inhalation or intranasal administration, and trachea/bronchial administration. The intrathecal administration may be performed using a shunt, an indwelling catheter, or a subcutaneous port. The dosing form of each of an agent for promoting delivery of the present aspect and a drug to be delivered to a nervous system may be independently selected from the above-described administration methods.

In one embodiment, an agent for promoting delivery of the present aspect is administered by transnasal administration, intravenous administration, subcutaneous administration, intraperitoneal administration, oral administration, inhalation administration, or intramuscular administration, and the drug to be delivered to a nervous system is administered by intrathecal administration.

In one embodiment, an agent for promoting delivery of the present aspect and a drug to be delivered to a nervous system may be administered simultaneously. For example, the below-described composition comprising both an agent for promoting delivery of the present aspect and a drug to be delivered to a nervous system may be administered.

In one embodiment, an agent for promoting delivery of the present aspect is administered before or after a drug to be delivered to a nervous system is administered. For example, an agent for promoting delivery of the present aspect and a drug to be delivered to a nervous system may be administered within 1 week, within 3 days, within 1 day, within 12 hours, within 6 hours, within 3 hours, or within 1 hour. For example, an agent for promoting delivery of the present aspect is intravenously administered or subcutaneously administered before a drug, and a drug to be delivered to a nervous system is intrathecally administered after an agent for promoting delivery.

As the dose of an agent for promoting delivery of the present aspect, an effective amount can be suitably selected in accordance with the kind of a drug to be administered in combination and the kind of a target disease. To a monkey or a human, a drug as an agent for promoting delivery may be administered in an amount of 0.01 mg or more, 0.1 mg or more, or 1 mg or more, e.g., 2 mg or more, 3 mg or more, 4 mg or more, 5 mg or more, 10 mg or more, 20 mg or more, 30 mg or more, 40 mg or more, 50 mg or more, 75 mg or more, 100 mg or more, 200 mg or more, 300 mg or more, 400 mg or more, or 500 mg or more, or may be administered in an amount of 0.01 mg to 10 g, 0.1 mg to 1 g, or 1 mg to 100 mg. To a mouse, the drug may be administered in an amount of 1 μg or more. The administration of the agent for promoting delivery may be single-dose administration or multiple-dose administration.

As the dose of a drug to be delivered to a nervous system, an effective amount can be suitably selected in accordance with the kind of the drug and the kind of a target disease. To a monkey or a human, a drug such as a nucleic acid medicine may be administered in an amount of 0.01 mg or more, 0.1 mg or more, or 1 mg or more, e.g., 2 mg or more, 3 mg or more, 4 mg or more, 5 mg or more, 10 mg or more, 20 mg or more, 30 mg or more, 40 mg or more, 50 mg or more, 75 mg or more, 100 mg or more, 200 mg or more, 300 mg or more, 400 mg or more, or 500 mg or more, or may be administered in an amount of 0.01 mg to 1000 mg, 0.1 mg to 200 mg, or 1 mg to 20 mg. To a mouse, the drug may be administered in an amount of 1 μg or more. The administration of a drug to be delivered to a nervous system may be single-dose administration or multiple-dose administration.

The dose of a drug to be delivered to a nervous system, such as a nucleic acid medicine, may be, e.g., 0.00001 mg/kg/day to 10000 mg/kg/day, or 0.001 mg/kg/day to 100 mg/kg/day. The administration of the drug may be single-dose administration or multiple-dose administration. In the case of multiple-dose administration, it may be administered daily or at appropriate time intervals (e.g., at intervals of 1 day, 2 days, 3 days, 1 week, 2 weeks, or 1 month), e.g., for 2 to 20 times. One dose of a drug such as a nucleic acid medicine may be, e.g., 0.001 mg/kg or more, 0.005 mg/kg or more, 0.01 mg/kg or more, 0.25 mg/kg or more, 0.5 mg/kg or more, 1.0 mg/kg or more, 2.0 mg/kg or more, 2.5 mg/kg or more, 3.0 mg/kg or more, 4.0 mg/kg or more, 5 mg/kg or more, 10 mg/kg or more, 20 mg/kg or more, 30 mg/kg or more, 40 mg/kg or more, 50 mg/kg or more, 75 mg/kg or more, 100 mg/kg or more, 150 mg/kg or more, 200 mg/kg or more, 300 mg/kg or more, 400 mg/kg or more, or 500 mg/kg or more. For example, any amount in the range of from 0.001 mg/kg to 500 mg/kg (e.g., 0.001 mg/kg, 0.01 mg/kg, 0.1 mg/kg, 1 mg/kg, 5 mg/kg, 10 mg/kg, 50 mg/kg, 100 mg/kg, or 200 mg/kg) may be selected as appropriate.

### (Effects)

Using an agent for promoting delivery of the present invention in combination with a drug such as a nucleic acid medicine makes it possible to enhance the efficiency of delivery of the drug dramatically. Accordingly, an agent for promoting delivery of the present invention can also be called an agent for augmenting delivery, an agent for aiding delivery, or the like.

In addition, the present invention provides a method comprising administering an agent for promoting delivery of the present invention to a subject, wherein the method enhances the efficiency of delivery of a drug to the central nervous system and/or the peripheral nervous system, compared with a subject to which an agent for promoting delivery of the present invention is not administered.

WO2017/150704 suggested that administering an agent for promoting the function of AQP4 promotes discharging amyloid-β out of the brain. Accordingly, the effect of dramatically increasing the amount of a nucleic acid medicine delivered into the brain in Examples herein is an effect unexpected from the conventional technology.

### <2. Pharmaceutical Composition>

A second aspect of the present invention is a pharmaceutical composition for treating the central nervous system disease or the peripheral nervous system disease. A pharmaceutical composition of the present aspect encompasses a therapeutically effective amount of a drug and an agent for promoting delivery of the first aspect.

### (Active Ingredient)

A pharmaceutical composition of the present aspect comprises a drug and an agent for promoting delivery as active ingredients. The configuration of each of the drug and the agent for promoting delivery is in accordance with the description of the first aspect, and the description is omitted here.

The amount (content) of each of a drug and an agent for promoting delivery that are comprised in a pharmaceutical composition of the present aspect varies depending on the kind of each of the drug and the agent for promoting delivery, the delivery site, the dosage form of the composition, the dose of the composition, and the kind of a carrier described below. Therefore, the amount may be determined as appropriate by taking the respective conditions into consideration. Usually, the amount may be adjusted so that an effective amount of the drug can be encompassed in a single dose of the composition. An “effective amount” refers to an amount that is necessary for the drug to function as an active ingredient, and to an amount that has little or no adverse side effect on a living body to which the amount is applied. This effective amount can vary depending on various conditions such as information on the subject, the administration route, and the number of administrations. Ultimately, the effective amount may be determined by the judgment of a physician, veterinarian, pharmacist, or the like. “Information on the subject” is various information on an individual of the living body to which the composition is applied. For example, when the subject is a human, the information comprises age, body weight, gender, dietary habit, health status, stage of progression or grade of severity of the disease, drug sensitivity, and presence of a combined drug.

### (Carrier)

The pharmaceutical composition of the present aspect may comprise a pharmaceutically acceptable carrier. A “pharmaceutically acceptable carrier” refers to an additive commonly used in the technical field of pharmaceutical preparation. Examples thereof include a solvent, a vegetable oil, a base, an emulsifier, a suspending agent, a surfactant, a pH adjuster, a stabilizer, an excipient, a vehicle, a preservative, a binder, a diluent, an isotonizing agent, a sedative, a bulking agent, a disintegrating agent, a buffering agent, a coating agent, a lubricant, a thickener, a dissolution aid, and other additives.

The solvent may be any of, e.g., water or other pharmaceutically acceptable aqueous solution, and a pharmaceutically acceptable organic solvent. Examples of an aqueous solution include a physiological saline, an isotonic solution comprising glucose or another additive, a phosphate-buffered saline, and a sodium acetate buffer solution. Examples of the additive include D-sorbitol, D-mannose, D-mannitol, sodium chloride, and further a nonionic surfactant at a low concentration, and polyoxyethylene sorbitan fatty acid ester.

The above carrier is used to avoid or decrease degradation of the drug and the agent for promoting delivery in vivo by an enzyme and the like, and additionally to facilitate formulation or administration, and to maintain the dosage form and drug efficacy. Therefore, it may be used as appropriate and as needed.

### (Dosage Form)

There is no particular restriction on the dosage form of the pharmaceutical composition of the present invention as long as the drug and the agent for promoting delivery are delivered to a target site without being inactivated by degradation or the like, and the pharmacological effect (an antisense effect on the expression of a target gene) of the active ingredient can be produced in vivo.

When the pharmaceutical composition of the present invention is intrathecally administered, the specific dosage form can be a dosage form appropriate for intrathecal administration. In this case, examples of a preferable dosage form include an injectable. An injectable can be formulated by mixing in an appropriate combination with the aforedescribed excipient, elixir, emulsifier, suspending agent, surfactant, stabilizer, pH adjuster, etc. in the form of a unit dose required according to the generally approved pharmaceutical practices.

There is no particular restriction on the specific shape and size of each of the above-mentioned dosage forms, as long as the respective dosage forms are within the ranges of dosage forms publicly known in the art. As to the manufacturing method of the pharmaceutical composition of the present aspect, it may be formulated according to the common procedure in the art.

### (Dosing Form and Dose)

The dosing form and dose of a pharmaceutical composition of the present aspect are not particularly limited, and are in accordance with the description of “(Agent for promoting delivery/Dosing form of drug)” in the first aspect.

The administration may be systemic or local. The administration route may be oral or parenteral administration. Specific examples of the parenteral administration include intrathecal administration (intraventricular administration, suboccipital puncture, or lumbar puncture), transnasal administration, intravenous administration, intraarterial administration, administration by blood transfusion, intraperitoneal administration, intraocular administration, intramuscular administration, subcutaneous administration (comprising implantable continuous subcutaneous administration), intradermal administration, intravesical administration, intravaginal administration, rectal administration, inhalation or intranasal administration, and trachea/bronchial administration. Because a site as a subject of application of the present invention is the central nervous system and/or the peripheral nervous system, intrathecal administration or transnasal administration, which is advantageous for delivery to the target site, is preferred. The composition can also be delivered to the central nervous system by crossing the blood-brain barrier, e.g., by intravenous administration, subcutaneous administration, intraperitoneal administration, oral administration, inhalation administration, or intramuscular administration.

### (Disease as Subject of Application)

A disease as a subject of application of the pharmaceutical composition is, e.g., the central nervous system disease or the peripheral nervous system disease. The subject can be a disease that can be controlled by delivering a drug comprised in a pharmaceutical composition of the present invention to the central nervous system and/or the peripheral nervous system. For example, in a case where the drug is a nucleic acid medicine, the disease may be a disease which may be related to a gene for which the antisense effect of the medicine can suppress or increase the expression amount of a target gene, a transcriptional product, or translation product, can inhibit the function of a transcriptional product or translation product of the gene, or can induce steric blocking, splicing switching, RNA editing, exon skipping, or exon inclusion.

The pharmaceutical composition can be used in an animal comprising a human as a subject. However, there is no particular restriction on the animal other than a human, a variety of domestic animals, domestic fowls, pets, laboratory animal, and the like can be subjects of some embodiments. The subject may be a subject in need of a decrease in the expression amount of a target transcriptional product in the central nervous system and/or the peripheral nervous system. The subject may also be a subject in need of treatment of a nervous disease, such as a brain disease.

In a case where the drug is a nucleic acid medicine, a disease to be treated can be the central nervous system disease or the peripheral nervous system disease associated with increased or decreased gene expression, particularly a disease (such as a tumor) associated with increased expression of a target transcriptional product or a target gene. Examples of the central nervous system disease include, but are not particularly limited to, brain tumor, Alzheimer's disease, Parkinson's disease, amyotrophic lateral sclerosis, multiple sclerosis, and Huntington's disease.

In one embodiment, a subject of a pharmaceutical composition of the present aspect can be Alzheimer's disease, cerebral infarction, brain tumor, demyelinating disease, epilepsy, and the central nervous system disease other than neuropathic pain.

The nervous system can be divided into the central nervous system and the peripheral nervous system. The central nervous system consists of the brain and the spinal cord. The brain comprises the cerebrum (cerebral cortex, cerebral white matter, and basal ganglion), the diencephalon (thalamus and subthalamic nucleus), the cerebellum (cerebellar cortex and cerebellar nucleus), and the brainstem (midbrain, substantia nigra, pons, and medulla oblongata). The spinal cord comprises the cervical cord, the thoracic cord, the lumbar cord, the sacral cord, and the coccygeal cord. Although the central nervous system herein may be any of these regions, it may be particularly the cerebral cortex (frontal lobe, temporal lobe, parietal lobe, or occipital lobe), the cerebellum, the striatum, the globus pallidus, the claustrum, the hippocampus, the parahippocampal gyrus, the brainstem, the cervical cord, the thoracic cord, or the lumbar cord. The peripheral nerve consists of a cranial nerve and a spinal nerve, and comprises a ventral root of the spinal cord, dorsal root, 1st to 12th cranial nerves, cauda equina, and dorsal root ganglion.

Herein, “the central nervous system disease” means a disease associated with any site in the central nervous system or the whole central nervous system. The central nervous system comprises the cerebrum (cerebral cortex, cerebral white matter, and basal ganglion), the diencephalon (thalamus and subthalamic nucleus), the cerebellum (cerebellar cortex and cerebellar nucleus), and the brainstem (midbrain, substantia nigra, pons, and medulla oblongata). The spinal cord comprises the cervical cord, the thoracic cord, the lumbar cord, the sacral cord, and the coccygeal cord. The central nervous system disease may be a disease associated with any of these sites, but may be particularly a disease associated with the cerebral cortex (frontal lobe, temporal lobe, parietal lobe, or occipital lobe), the cerebellum, the striatum, the globus pallidus, the claustrum, the hippocampus, the parahippocampal gyrus, the brainstem, the cervical cord, the thoracic cord, or the lumbar cord.

For example, in a treatment of Alzheimer's disease, drug delivery to the hippocampus and/or the parietal lobe may be effective. In a treatment of frontotemporal dementia (FTD) (frontotemporal lobar degeneration (FTLD), semantic dementia (SD), or progressive nonfluent aphasia (PNFA)), and Pick disease, drug delivery to the frontal lobe, the temporal lobe, and/or the substantia nigra may be effective. In a treatment of Parkinson's disease dementia, drug delivery to the occipital lobe, the substantia nigra, and/or the striatum may be effective. In a treatment of Parkinson's disease, drug delivery to the substantia nigra and/or the striatum may be effective. In a treatment of corticobasal degeneration (CBD), drug delivery to the frontal lobe, the parietal lobe, the basal ganglion, and/or the substantia nigra may be effective. In a treatment of progressive supranuclear palsy (PSP), drug delivery to the frontal lobe, the basal ganglion, and/or the substantia nigra may be effective. In a treatment of amyotrophic lateral sclerosis, drug delivery to the frontal lobe, the parietal lobe, the basal ganglion, and/or the substantia nigra may be effective. In a treatment of spinocerebellar degeneration (SCD) SCA1 type through SCA34 type, drug delivery to the brainstem and/or the cerebellum may be effective. In a treatment of dentatorubral-pallidoluysian atrophy (DRPLA), drug delivery to the basal ganglion, the brainstem, and/or the cerebellum may be effective. In a treatment of spinal and bulbar muscular atrophy (SBMA), drug delivery to the brainstem and/or the spinal cord may be effective. In a treatment of Friedreich's ataxia (FA), drug delivery to the brainstem and/or the cerebellum may be effective. In a treatment of Huntington's disease, drug delivery to the striatum, the frontal lobe, the parietal lobe, and/or the basal ganglion may be effective. In a treatment of a prion disease (mad cow disease, GSS), drug delivery to the cerebral cortex, the cerebral white matter, the basal ganglion, and/or the substantia nigra may be effective. In the treatment of cerebral white matter encephalopathy, drug delivery to the cerebral white matter may be effective. In a treatment of encephalitis (viral, bacterial, fungal, or tuberculous) or meningitis (viral, bacterial, fungal, or tuberculous), drug delivery to the whole brain may be effective. In a treatment of metabolic encephalopathy, toxic encephalopathy, trophopathic encephalopathy, drug delivery to the whole brain may be effective. In a treatment of cerebral white matter encephalopathy, drug delivery to the cerebral white matter may be effective. In a treatment of cerebral infarction, cerebral hemorrhage, subarachnoid hemorrhage, moyamoya disease, anoxic encephalopathy, drug delivery to the whole brain may be effective. In a treatment of cerebral white matter encephalopathy, drug delivery to the cerebral white matter may be effective. In a treatment of diffuse axonal injury, drug delivery to the cerebral white matter may be effective. In a treatment of head trauma, drug delivery to the whole brain may be effective. In a treatment of multiple sclerosis (MS) or neuromyelitis optica (NMO), drug delivery to the cerebral white matter, the cerebral cortex, the optic nerve, and/or the spinal cord may be effective. In a treatment of myotonic dystrophy (DM1, DM2), drug delivery to the skeletal muscle, the cardiac muscle, the cerebral cortex, and/or the cerebral white matter may be effective. In a treatment of hereditary spastic paraparesis (HSP), drug delivery to the parietal lobe and/or the spinal cord may be effective. In a treatment of Fukuyama muscular dystrophy, drug delivery to the skeletal muscle, the cerebral cortex, and/or the cerebral white matter may be effective. In a treatment of dementia with Lewy bodies (DLB), drug delivery to the substantia nigra, the striatum, the occipital lobe, the frontal lobe, and/or the parietal lobe may be effective. In a treatment of multiple system atrophy (MSA), drug delivery to the striatum, the basal ganglia, the cerebellum, the substantia nigra, the frontal lobe, and/or the temporal lobe may be effective. In a treatment of Alexander's disease drug delivery to the cerebral white matter may be effective. In a treatment of CADASIL and CARASIL, drug delivery to the cerebral white matter may be effective.

Herein, “the peripheral nervous system disease” means a disease associated with any site in the peripheral nervous system or the whole peripheral nervous system. The peripheral nerve consists of a cranial nerve and a spinal nerve, and comprises a ventral root of the spinal cord, dorsal root, 1st to 12th cranial nerves, cauda equina, and dorsal root ganglion. The peripheral nervous system disease may be a disease associated with any of these sites, and may be particularly a disease associated with a ventral root of the spinal cord, dorsal root, 1st to 12th cranial nerves, cauda equina, or dorsal root ganglion.

### (Effects)

A pharmaceutical composition of the present invention comprises an agent for promoting delivery in addition to a therapeutically effective amount of a drug, thereby enhancing the efficiency of delivery of the drug to the central nervous system and/or the peripheral nervous system dramatically, and, on this basis, can increase the drug efficacy.

In addition, the present invention also provides a method for treating and/or preventing a disease such as the central nervous system disease or the peripheral nervous system disease, comprising administering (e.g., intrathecally administering) a pharmaceutical composition of the present aspect to a subject, such as a human.

Also provided is use of an agent for promoting delivery of the first aspect or a pharmaceutical composition of the second aspect in the production of a medicine.

### EXAMPLES

The present invention is described below in more detail by means of Examples. However, the technical scope of the present invention is not limited to these Examples.

### Example 1: Identification of Novel Compound that Increases Efficiency of Delivery of Antisense Nucleic Acid to Nervous System and Gene Suppression Effect of Nucleic Acid in Nervous System

### (Purpose)

In order to identify a novel compound capable of increasing, in a nervous system, the gene suppression effect based on an antisense nucleic acid targeted at the Malat1 gene (hereinafter referred to as “Malat1-targeting ASO”), TGN-073 as a candidate compound, together with the ASO, is administered to the cerebral ventricle of a mouse. Investigated through an in vivo experiment are: the efficiency of delivery of an antisense nucleic acid to the central nervous system; and the influence of the TGN-073 coadministration on the target gene suppression effect in the central nervous system. As the ASO in this Example, the Malat1-targeting ASO is used.

### (Method)

### (1) Synthesis of TGN-073

As a candidate compound, N-(3-benzyloxypyridin-2-yl)-benzene-sulfonamide represented by the following formula (I) was synthesized.

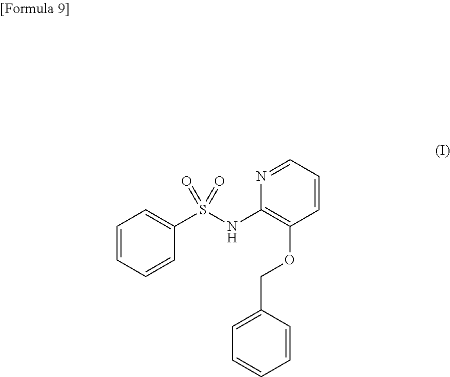 [SMILES: O=S(=O)(Nc1ncccc1OCc1ccccc1)c1ccccc1]

The compound represented by the formula (I) can also be referred to as 2-(phenylsulfonamido)-3-benzyloxypyridine. In addition, the compound represented by the formula (I) is referred to as “TGN-073” in the present inventors' past literature (Huber V. J. et al., NeuroReport, 2018, 29 (9): 697-703), and is referred to as “2A” in WO2017/150704. Herein, the compound represented by the formula (I) and a salt thereof are referred to as “TGN-073”.

TGN-073 was synthesized in accordance with the method of producing the compound 2A, the method described in WO2017/150704. Specifically, 2-amino-3-benzyloxypyridine (1.50 g, 7.50 mmol) was added to a 50-mL-capacity round bottom flask having dry dichloromethane (35 mL) added thereto. Subsequently, an aqueous solution of 2,6-lutidine (2.62 mL, 22.5 mmol) was added to the solution under stirring, using a syringe, and the reactor was charged with argon. Subsequently, benzenesulfonyl chloride (1.05 mL, 8.25 mmol) was added to the solution under stirring, using a syringe. Subsequently, the reaction was performed with stirring under an argon atmosphere at room temperature for 72 hours. Subsequently, the resulting yellow solution was washed with a 10% citric acid solution (2×, 25 mL) and a saturated sodium carbonate solution (2×, 25 mL). Subsequently, the resulting solution was dried using magnesium sulfate, filtered, and underwent water evaporation to yield a dark red semisolid crystal. Subsequently, the resulting dark red crystal was washed with hexane. Subsequently, the resulting crystal underwent elution with a 33% ethyl acetate/hexane solution through a flash chromatography column used with silica gel (WAKOGEL 300), whereby a white crystal (the compound TGN-073)(the yield by weight, 0.309 g; the yield by ratio, 13.5%) was obtained. The synthetic route of TGN-073 is illustrated below.

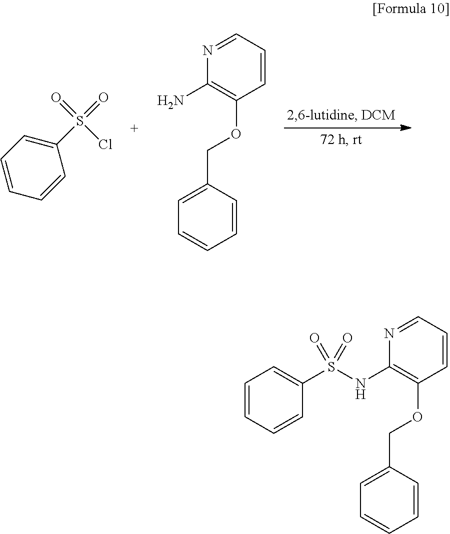 [SMILES: O=S(=O)(Cl)c1ccccc1 ; Nc1ncccc1OCc1ccccc1 ; O=S(=O)(Nc1ncccc1OCc1ccccc1)c1ccccc1]

In this regard, when TGN-073 was administered in the below-described Examples, a sodium salt of a compound represented by the above-described formula (I) was used. The sodium salt of a compound represented by the formula (I) is represented by the following formula (II).

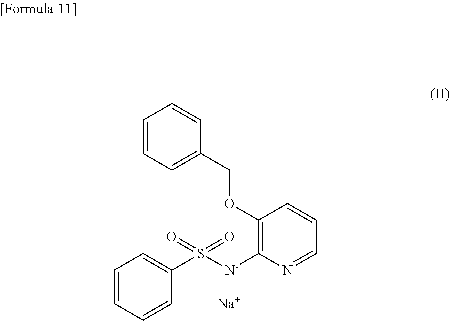 [SMILES: O=S(=O)([N-]c1ncccc1OCc1ccccc1)c1ccccc1 ; [Na+]]

### (2) Preparation of Nucleic Acids

The base sequence and chemical modification of the ASO used in this Example are shown in Table 1 below.

TABLE 1
Antisense nucleic acid targeted at Malat1 gene (Malat1-targeting ASO)

| | Sequence (5′-3′) | SEQ ID NO |
|---|---|---|
| ASO | 5(L)^{∧}T(L)^{∧}A(L)^{∧}g^{∧}t^{∧}t^{∧}c^{∧}a^{∧}c^{∧}t^{∧}g^{∧}a^{∧}a^{∧} | 1 |
| | T(L)^{∧}G(L)^{∧}5(L) | |

Lower case letter: DNA; Upper case letter (L): LNA; 5(L): 5-methylcytosine LNA; ^{∧}: phosphorothioate linkage

The ASO used in this Example is an LNA/DNA gapmer antisense nucleic acid targeting a metastasis associated lung adenocarcinoma transcript 1 (Malat1) non-coding RNA of a mouse, has a base sequence complementary to part of the Malat1 ncRNA, and has a structure wherein three LNA nucleosides at the 5′ end, three LNA nucleosides at the 3′ end, and ten DNA nucleosides therebetween are linked via a phosphorothioate linkage. All the oligonucleotides were synthesized by GeneDesign Inc. (Osaka, Japan) on consignment.

### (3) In Vivo Experiment

Seven-week-old female ICR mice anesthetized with 2.5 to 4% isoflurane were fixed to a brain stereotaxic apparatus. Then, skin between the ears was cut open 2 to 3 cm anteroposteriorly, and perforated 1 mm leftward and 0.2 mm posteriorly from the bregma using a 1 mm diameter drill. A Hamilton syringe was filled with the nucleic acid agent. Through the perforated site, a needle was inserted approximately 3 mm deep. The ASO at a dose of 0.95 nmol (5 μg) per mouse was administered into the left lateral ventricle (n=4 to 7/group). The group to which ASO and TGN-073 were coadministered (hereinafter, the group is referred to as the “ASO/TGN-073 coadministration group”) means that ASO mixed with TGN-073 was administered. The group to which only ASO was administered (hereinafter, the group is referred to as the “ASO single administration group”) means that only ASO was administered without being mixed with TGN-073. The dose of TGN-073 in the ASO/TGN-073 coadministration group was 0.25 mg per mouse. After the intraventricular administration, the skin was sutured with a nylon thread. In addition, mice to which only TGN-073 was administered (hereinafter, the mice are referred to as the “TGN-073 administration group”) and mice to which only PBS was administered (hereinafter, the mice are referred to as the “PBS administration group”) were produced as controls.

### (4) Evaluation of Gene Suppression Effect

A left hippocampus was extirpated from each of the mice at 3 hours or 7 days after the intraventricular administration. Using an IsogenI kit (GeneDesign Inc.), RNA was extracted from the left hippocampus extirpated. A cDNA was synthesized using Transcriptor Universal cDNA Master, DNase (Roche Diagnostics) in accordance with the protocol.

Next, quantitative RT-PCR was performed with TaqMan (Roche Applied Science). Primers used in quantitative RT-PCR were products designed and manufactured by Thermo Fisher Scientific Inc. (formerly known as Life Technologies Corp.) on the basis of the various numbers of genes. Amplification conditions (temperature and time) were as follows: one cycle—95 °C for 15 seconds, 60 °C for 30 seconds, and 72 °C for 1 second—was repeated 40 times. The ratio of the expression amount of Malat1 RNA to the expression amount of Actb mRNA (internal standard gene) was calculated, and a value standardized with respect to the value of the PBS-treated group was determined as a relative Malat1 RNA level.

The intracerebral ASO concentration (the amount of the nucleic acid delivered to the brain) was measured by extracting RNA from the left hippocampus extirpated in the same manner as described above, and performing quantitative RT-PCR using a primer pair for amplifying ASO and TaqMan Small RNA Assay (Roche Applied Science). The intracerebral ASO concentration was obtained by calculating the ASO content relative to U6 snRNA (an internal standard gene).

### (Results)

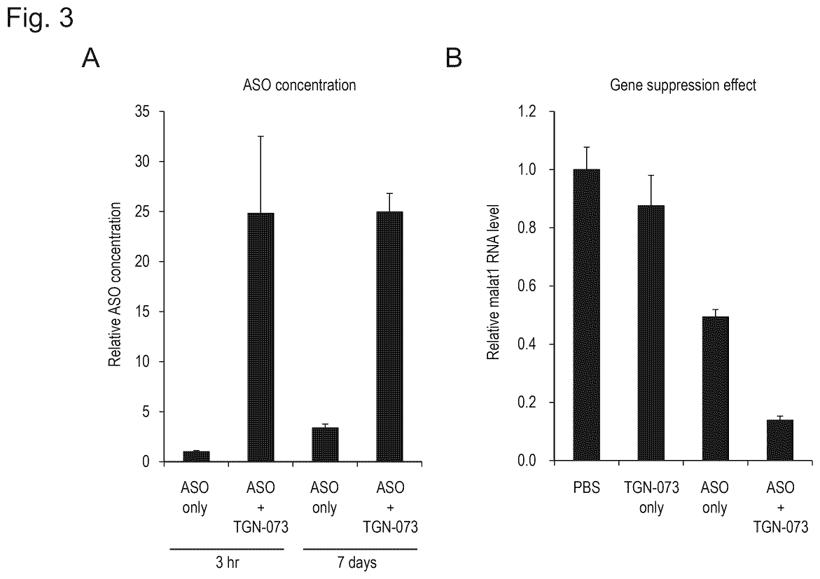

FIG. 3A shows the ASO concentration in the hippocampus at 3 hours or 7 days after the intraventricular administration. The ASO/TGN-073 coadministration group exhibited a dramatic increase in the intracerebral ASO concentration (the amount of the nucleic acid delivered to the brain), compared with the ASO single administration group.

FIG. 3B shows the Malat1 RNA expression level in the hippocampus at 7 days after the intraventricular administration. With the ASO/TGN-073 coadministration group, the suppression effect on the Malat1 gene was markedly enhanced, compared with the ASO single administration group.

The above-described results have revealed the surprising effect by which the TGN-073 coadministration markedly improved the efficiency of delivery of the ASO to the central nervous system, and dramatically augmented the target gene suppression effect in the central nervous system.

### Example 2: Dose Dependency of ASO Intraventricularly Administered Together with TGN-073

### (Purpose)

In order to quantitatively evaluate the effect of TGN-073, which effect was discovered in Example 1, ASO at various concentrations is intraventricularly administered together with TGN-073 to investigate, through an in vivo experiment, the efficiency of delivery of ASO to the central nervous system and the target gene suppression effect in the central nervous system.

### (Method and Results)

In this Example, the ASO targeted at Malat1, and used in Example 1 (the ASO described in Table 1) was used. Performed in accordance with Example 1 were: the preparation of a nucleic acid; an in vivo experiment; and the evaluation of the intracerebral ASO concentration (the amount of the nucleic acid delivered to the brain); and the target gene suppression effect. In this Example, however, the dose of ASO per mouse was 1.25 μg, 5 μg, and 20 μg for the ASO single administration group, and 0.075 μg, 0.3 μg, and 1.25 μg for the ASO/TGN-073 coadministration group.

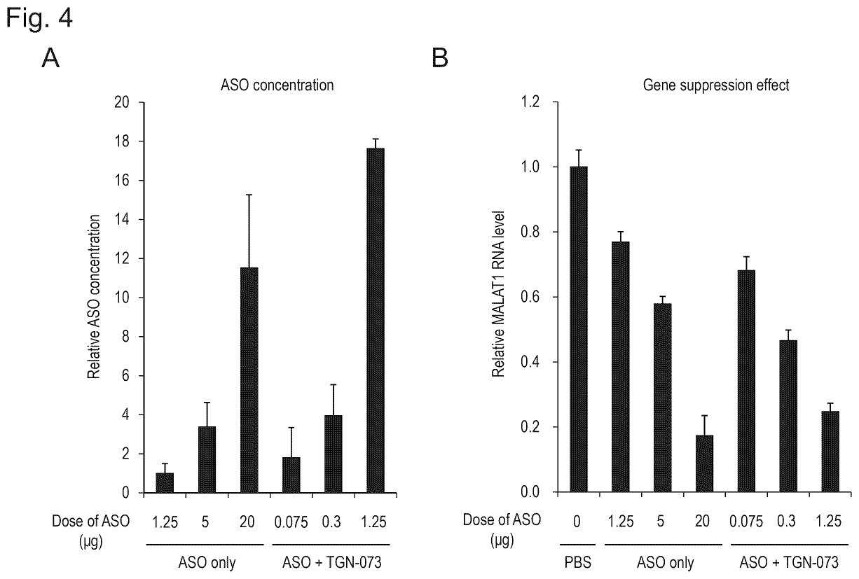

FIG. 4A shows the ASO concentration in the hippocampus at 7 days after the intraventricular administration of the nucleic acid agent. At a concentration approximately 16 times to 20 times lower than the ASO single administration group, the ASO/TGN-073 coadministration group achieved the same amount of delivery to the brain as the ASO single administration group.

FIG. 4B shows the Malat1 RNA expression level in the hippocampus at 7 days after the intraventricular administration of the nucleic acid agent. At a concentration approximately 16 times to 20 times lower than the ASO single administration group, the ASO/TGN-073 coadministration group achieved the same gene suppression effect as the ASO single administration group.

The above-described results have revealed that the TGN-073 coadministration enhanced, approximately 16 times to 20 times better, the amount of delivery of the ASO to the central nervous system and the target gene suppression effect based on the ASO in the central nervous system.

### Example 3: Effect of TGN-073 Coadministration in Intraventricular Administration of Mapt-Targeting ASO

### (Purpose)

An ASO targeted at the Mapt gene (hereinafter referred to as the “Mapt-targeting ASO”) is intraventricularly administered together with TGN-073. Investigated through an in vivo experiment are: the influence of the TGN-073 coadministration on the target gene suppression effect in the central nervous system; and the central nervous system toxicity.

### (Method)

The base sequence and chemical modification of the ASO used in this Example are shown in Table 2 below.

TABLE 2
Antisense nucleic acid targeted at Mapt gene (Mapt-targeting ASO)

| | Sequence (5′-3′) | SEQ ID NO |
|---|---|---|
| ASO | A(L)^{∧}T(L)^{∧}A(L)^{∧}g^{∧}t^{∧}c^{∧}a^{∧}c^{∧}t^{∧}c^{∧}t^{∧}g^{∧}g^{∧} | 2 |
| | T(L)^{∧}G L)^{∧}A(L) | |

Lower case letter: DNA; Upper case letter (L): LNA; ^{∧}: phosphorothioate linkage

The ASO used in this Example is an LNA/DNA gapmer antisense nucleic acid targeting a microtubule-associated protein tau (Mapt) mRNA of a mouse, has a base sequence complementary to part of the Mapt mRNA, and has a structure wherein three LNA nucleosides at the 5′ end, three LNA nucleosides at the 3′ end, and ten DNA nucleosides therebetween are linked via a phosphorothioate linkage.

On the ASO described in Table 2, the preparation of the nucleic acid, an in vivo experiment, and the evaluation of the target gene suppression effect were performed by the same methods as in Example 1. In this Example, however, the dose of the nucleic acid agent per mouse was 9.4 nmol (50 μg).

By the below-described methods, evaluation of central nervous system toxicity and evaluation of motor function were performed on mice after intraventricular administration.

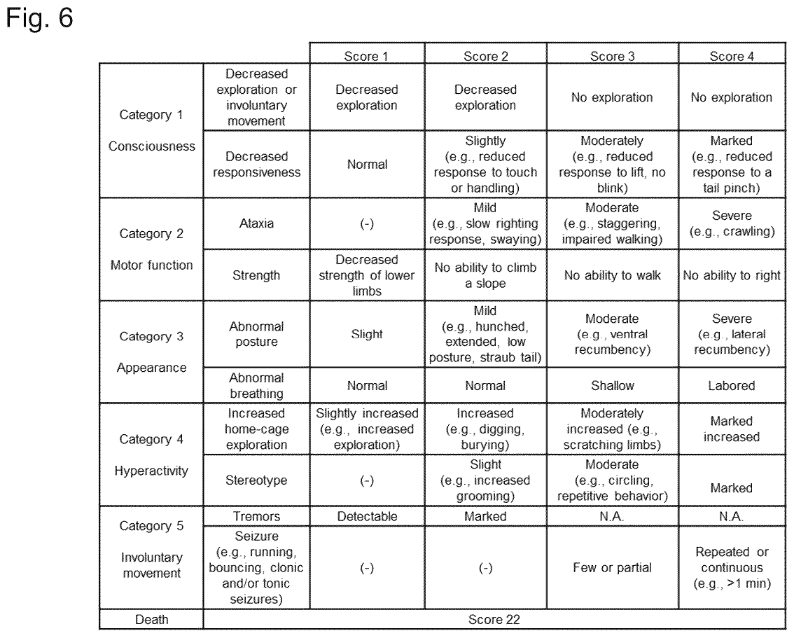

In the evaluation of central nervous system toxicity, the behavior was evaluated, using the scoring system shown in FIG. 6, at 30 minutes, 1 hour, and 2 hours after the intraventricular administration. According to the scoring system shown in FIG. 6, the behaviors classified into five categories were evaluated (Categories 1 to 5 in FIG. 6). Each category comprises two behavior evaluation items. Each behavior evaluation item is rated in five stages from 0 to 4 points (Scores 0 to 4 in FIG. 6). A normal behavior is given 0 point, and higher toxicity is given a higher score. In each category, the higher score in the two behavior evaluation items is adopted as the score for the category. The total value of the scores for the five categories is defined as an acute tolerability score (0 to 20 points).

To evaluate the motor function, an open-field test was performed at each point of time after the intraventricular administration. Specifically, a mouse was placed in the center of a cage (50 cm in width×50 cm in diameter×40 cm in height), and the track of the mouse was recorded for 5 minutes. On the basis of the data recorded, the total maximum moving speed (m/s) and the moving time(s) were measured using the video tracking software (ANY-maze).

### (Results)

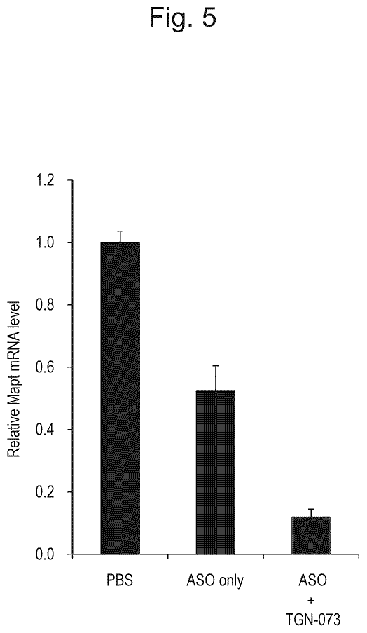

FIG. 5 shows the Mapt RNA expression level in the hippocampus at 7 days after the intraventricular administration of the nucleic acid agent. The ASO/TGN-073 coadministration group exhibited a marked augmentation of the target gene suppression effect, compared with the ASO single administration group.

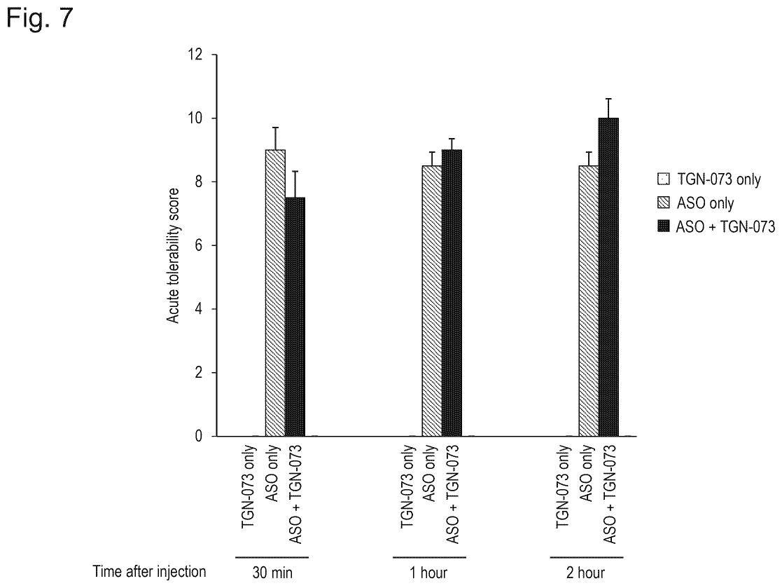

FIG. 7 shows the results of evaluation of the central nervous system toxicity. The TGN-073 single administration group exhibited an acute tolerability score of 0, and exhibited no toxicity, at any point in time of 30 minutes, 1 hour, and 2 hours after the intraventricular administration. In addition, the ASO/TGN-073 coadministration group exhibited substantially the same acute tolerability score as the ASO single administration group.

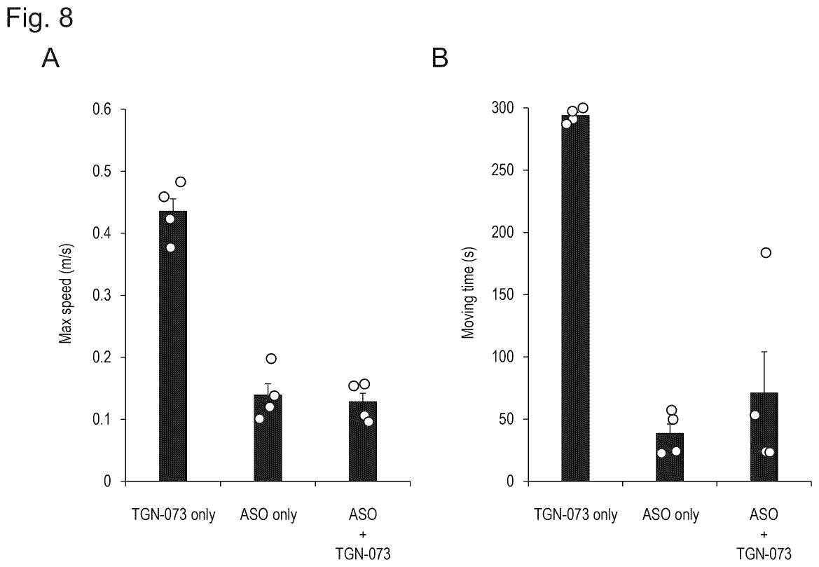

FIG. 8 shows the results of evaluation of the motor function in an open-field test at 1 hour after the administration. The ASO/TGN-073 coadministration group exhibited substantially the same maximum moving speed (FIG. 8A) and the same moving time (FIG. 8B) as the ASO single administration group.

The above-described results have revealed that the TGN-073 coadministration dramatically increased the target gene suppression effect based on ASO in the central nervous system, and, on the other hand, caused no recognizable augmentation of neurotoxicity at all.

### Example 4: Effect of TGN-073 Coadministration in Intraventricular Administration of 2′-O-MOE-Modified ASO

### (Purpose)

TGN-073 is coadministered in the intraventricular administration of an ASO that is the same Malat1-targeting ASO used in Example 1 except that a 2′-MOE modification as a different chemical modification of the nucleic acid was introduced in the wing region. Investigated through an in vivo experiment is: the influence of the TGN-073 coadministration on the target gene suppression effect in the central nervous system.

### (Method)

The base sequence and chemical modification of the ASO used in this Example are shown in Table 3 below.

TABLE 3
2′-O-MOE-modified ASO targeted at Malat1 gene

| | Sequence (5′-3′) | SEQ ID |
|---|---|---|
| ASO | G (m)^{∧}G(m)^{∧}G(m)^{∧}T(m)^{∧}5(m)^{∧}a^{∧}g^{∧}c^{∧}t^{∧}g^{∧}c^{∧}c^{∧}a^{∧}a^{∧}t | 3 |
| | ^{∧}G(m)^{∧}5(m)^{∧}T(m)^{∧}A(m)^{∧}G(m) | |

Lower case letter: DNA; Upper case letter (m): 2′-O-MOE-RNA; 5(m): 5-methylcytosine 2′-O-MOE-RNA; ^{∧}: phosphorothioate linkage

The ASO used in this Example is a 2′-O-MOE-RNA/DNA gapmer antisense nucleic acid targeting the Malat1 non-coding RNA, has a base sequence complementary to part of the Malat1 ncRNA, and has a structure wherein five 2′-O-MOE-RNA nucleosides at the 5′ end, five 2′-O-MOE-RNA nucleosides at the 3′ end, and ten DNA nucleosides therebetween are linked via a phosphorothioate linkage.

On the ASO described in Table 3, the preparation of the nucleic acid, an in vivo experiment, and the evaluation of the target gene suppression effect were performed by the same methods as in Example 1. In this Example, however, the amount of the nucleic acid agent administered per mouse was 1.7 nmol (12.5 μg)/mouse.

### (Results)

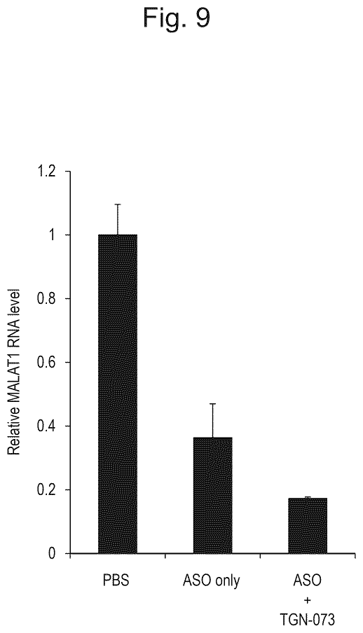

FIG. 9 shows the Malat1 RNA expression level in the hippocampus at 7 days after the intraventricular administration. The ASO/TGN-073 coadministration group exhibited an augmentation of the target gene suppression effect, compared with the ASO single administration group.

The above-described results have revealed that, independent of the kind of the chemical modification in the antisense nucleic acid, the TGN-073 coadministration dramatically enhanced the target gene suppression effect in the brain.

### Example 5: Effect of TGN-073 Coadministration in Intraventricular Administration of Heteroduplex Nucleic Acid

### (Purpose)

TGN-073 is coadministered in the intraventricular administration of a heteroduplex oligonucleotide (hereinafter referred to as “HDO”) comprising: a first nucleic acid strand consisting of the single-stranded ASO used in Example 1; and a second nucleic acid strand having a base sequence complementary to the first nucleic acid strand. Investigated through an in vivo experiment are: the influences of the TGN-073 coadministration on the amount of the nucleic acid delivered to the central nervous system and on the target gene suppression effect in the central nervous system.

### (Method)

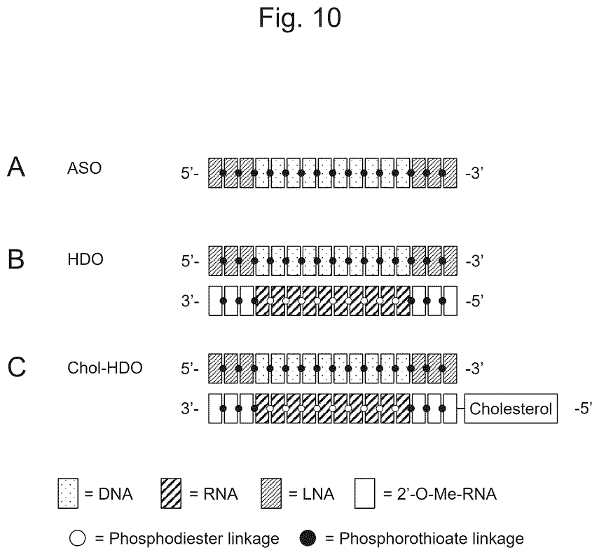

The base sequences and chemical modifications of the single-stranded nucleic acid and the double-stranded nucleic acids used in this Example are shown in FIG. 10 and Table 4 below.

TABLE 4
Single-stranded and double stranded nucleic acid agents targeted at Malat1 gene

| | | Sequence (5′-3′) | SEQ ID |
|---|---|---|---|
| ASO | | 5(L)^{∧}T(L)^{∧}A(L)^{∧}g^{∧}t^{∧}t^{∧}c^{∧}a^{∧}c^{∧}t^{∧}g^{∧}a^{∧}a^{∧} | 1 |
| | | T(L)^{∧}G(L)^{∧}5(L) | |
| HDO | ASO | 5(L)^{∧}T(L)^{∧}A(L)^{∧}g^{∧}t^{∧}t^{∧}c^{∧}a^{∧}c^{∧}t^{∧}g^{∧}a^{∧}a^{∧} | 1 |
| | | T(L)^{∧}G(L)^{∧}5(L) | |
| | CRNA | G(M)^{∧}C(M)^{∧}A(M)^{∧}UUCAGUGAAC^{∧}U(M)^{∧}A(M) | 4 |
| | | ^{∧}G(M) | |
| Chol-HDO | ASO | 5(L)^{∧}A(L)^{∧}A(L)^{∧}g^{∧}t^{∧}t^{∧}c^{∧}a^{∧}c^{∧}t^{∧}g^{∧}a^{∧}a^{∧} | 1 |
| | | T(L)^{∧}G(L)^{∧}5 L) | |
| | Chol-cRNA | Chol-G(M)^{∧}C(M)^{∧}A(M)^{∧}UUCAGUGAAC^{∧}U(M) | 5 |
| | | ^{∧}A(M)^{∧}G(M) | |

Lower case letter: DNA; Upper case letter: RNA; Upper case letter (L): LNA; 5(L): 5-methylcytosine LNA; Upper case letter (M): 2′-O-Me-RNA; ^{∧}: phosphorothioate linkage; Chol: cholesterol

The ASOs described in Table 4 were prepared by the same methods as in Example 1.

The heteroduplex oligonucleotide (HDO) used in this Example comprised the above-described ASO as the first nucleic acid strand, and the second nucleic acid strand had a sequence complementary to the first nucleic acid strand.

A heteroduplex oligonucleotide having cholesterol bound to the 5′ side thereof (Chol-HDO) comprised the above-described ASO as the first nucleic acid strand. Cholesterol was linked with the 5′ end of the second nucleic acid strand.

On the ASO, HDO, and Chol-HDO described in Table 4, an in vivo experiment, the measurement of the ASO concentration in the central nervous system, and the evaluation of the target gene suppression effect were performed by the same methods as in Example 1. In this Example, however, the amount of the nucleic acid agent administered per mouse was 0.95 nmol.

### (Results)

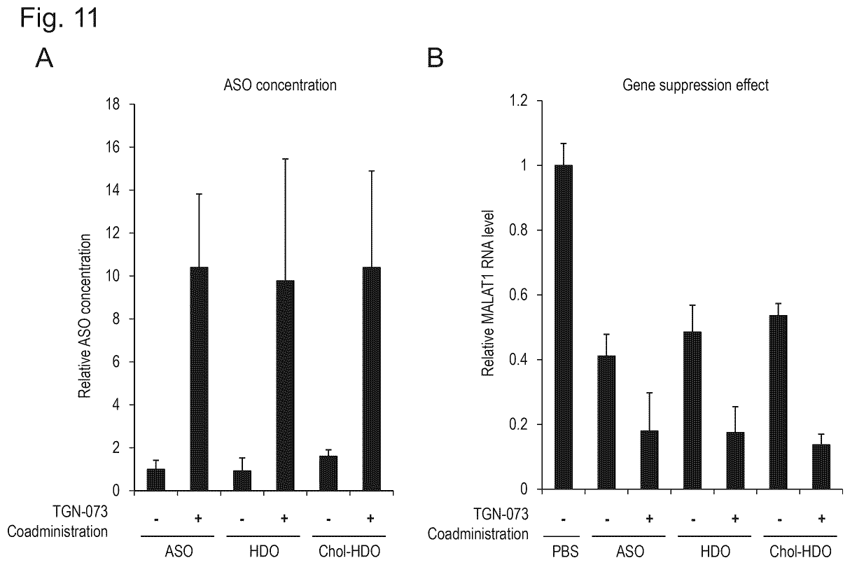

FIG. 11A shows the ASO concentration in the hippocampus at 7 days after the intraventricular administration. Any of the groups that underwent the TGN-073 coadministration together with ASO, HDO, and Chol-HDO exhibited a dramatic increase in the ASO concentration in the central nervous system, compared with the groups that underwent the administration of the nucleic acid agent alone without TGN-073 coadministration.

FIG. 11B shows the Malat1 RNA expression level in the hippocampus at 7 days after the intraventricular administration. Any of the TGN-073 coadministration groups exhibited a marked enhancement in the target gene suppression effect in the central nervous system, compared with the groups that underwent the administration of each nucleic acid agent alone without TGN-073 coadministration.

The above-described results have revealed that, not only with the single-stranded nucleic acid agent but also with the double-stranded nucleic acid agent, the TGN-073 coadministration exhibited a dramatic enhancement in the amount of the nucleic acid delivered to the central nervous system and the target gene suppression effect in the central nervous system.

### Example 6: Effect of TGN-073 Coadministration in Intraventricular Administration of siRNA

### (Purpose)

Together with TGN-073, siRNA is administered intraventricularly. Investigated through an in vivo experiment is: the influence of the TGN-073 coadministration on the target gene suppression effect in the central nervous system.

### (Method)

The base sequences and chemical modifications of the siRNAs used in this Example are shown in Table 5 below.

TABLE 5
siRNA targeted at Sod1 gene

| | | Sequence (5′-3′) | SEQ ID NO |
|---|---|---|---|
| siRNA | siRNA-s | C(M)^{∧}A(M)^{∧}U(M)U(M)U(M)A(F)A(M)U(F) | 6 |
| | | C(F)C(F)U(M)C(M)A(M)C(M)U(M)C(M)U(M) | |
| | | A(M)^{∧}A(M)^{∧}A(M) | |
| | siRNA-as | U(V)^{∧}U(F)^{∧}U(M)A(M)G(M)A(F)G(M)U(F) | 7 |
| | | G(F)A(M)G(M)G(M)A(M)U(F)U(M)A(F)A(M) | |
| | | A(M)A(M)U(M)G(M)^{∧}A(M)^{∧}G(M) | |

Upper case letter (F): 2′-fluoro RNA; Upper case letter (M): 2′-O-Me-RNA; ^{∧}: phosphorothioate linkage U(V): 5′-(E)-Vinylphosphonate(VP)-2′-O-Me-RNA having an uracil base

The siRNAs used in this Example are siRNAs targeted at copper-zinc superoxide dismutase 1 (Sod1) mRNA. On the siRNAs described in Table 5, an in vivo experiment and the evaluation of the target gene suppression effect were performed by the same methods as in Example 1. In this Example, however, the amount of the nucleic acid agent administered per mouse was 7.0 nmol (100 μg)/mouse.

### (Results)

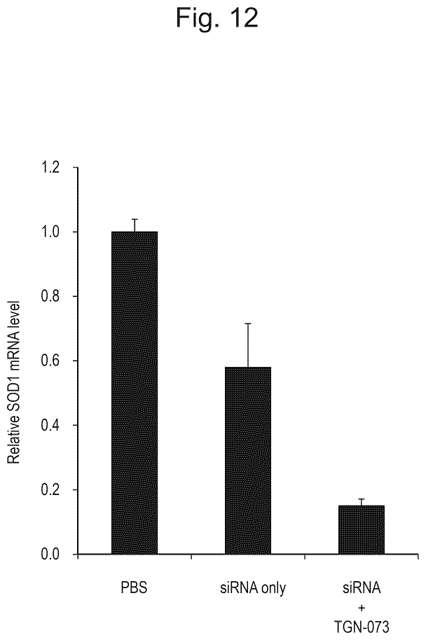

FIG. 12 shows the Sod1 mRNA expression level in the hippocampus at 7 days after the intraventricular administration. The TGN-073 coadministration group exhibited a marked augmentation of the target gene suppression effect in the brain, compared with the siRNA single administration group.

The above-described results have revealed that, in the intraventricular administration of siRNA, the TGN-073 coadministration exhibited a dramatic enhancement in the target gene suppression effect in the central nervous system.

### Example 7: TGN-073 Derivative

### (Purpose)

To study the effect of a TGN-073 derivative, TGN-073-mes as a TGN-073 derivative is coadministered in the intraventricular administration of an ASO targeted at the Mapt gene. Investigated through an in vivo experiment are: the influences on the amount of an ASO delivered to the central nervous system and on the target gene suppression effect in the central nervous system.

### (Method)

The base sequence and chemical modification of the ASO used in this Example are shown in Table 6 below.

TABLE 6
Antisense nucleic acid targeted at Mapt gene (Mapt-targeting ASO)

| | Sequence (5′-3′) | SEQ ID NO |
|---|---|---|
| ASO | A(L)^{∧}T(L)^{∧}T(L)^{∧}t^{∧}c^{∧}c^{∧}a^{∧}a^{∧}a^{∧}t^{∧}t^{∧}c^{∧}a^{∧} | 8 |
| | 5(L)^{∧}T(L)^{∧}T(L) | |

Lower case letter: DNA; Upper case letter (L): LNA; 5(L): 5-methylcytosine LNA; ^{∧}: phosphorothioate linkage

The ASO used in this Example is an LNA/DNA gapmer antisense nucleic acid targeted at a Mapt mRNA of a mouse, has a base sequence complementary to part of the Mapt mRNA, and has a structure wherein three LNA nucleosides at the 5′ end, three LNA nucleosides at the 3′ end, and ten DNA nucleosides therebetween are linked via a phosphorothioate linkage.

TGN-073-mes coadministered with ASO in this Example is a derivative of TGN-073X represented by the above-described formula (I). The chemical structure of the derivative is represented by the following formula (III).

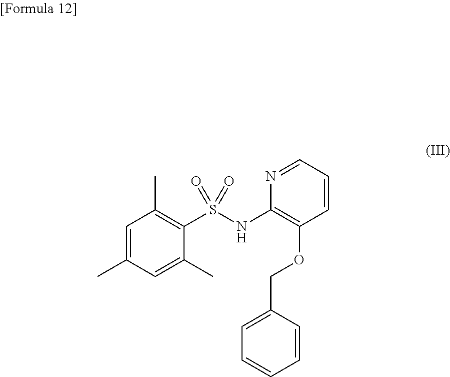 [SMILES: Cc1cc(C)c(S(=O)(=O)Nc2ncccc2OCc2ccccc2)c(C)c1]

Herein, the compound represented by the formula (III) and a salt thereof are referred to as “TGN-073-mes”. In this regard, when TGN-073-mes was administered in the below-described Examples, a sodium salt of a compound represented by the formula (III) was used. The sodium salt of a compound represented by the formula (III) is represented by the following formula (VII).

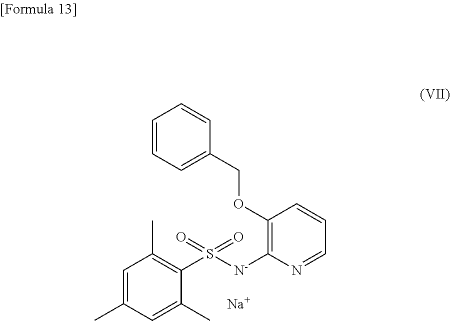 [SMILES: Cc1cc(C)c(S(=O)(=O)[N-]c2ncccc2OCc2ccccc2)c(C)c1 ; [Na+]]

On the ASO described in Table 6, the preparation of the nucleic acid, an in vivo experiment, and the evaluation of the ASO concentration in the central nervous system and the target gene suppression effect were performed by the same methods as in Example 1. In this Example, however, the amount of the nucleic acid agent administered per mouse was 9.4 nmol (50 μg). In addition, the ASO/TGN-073-mes coadministration group underwent intraventricular administration of TGN-073-mes mixed at a dose of 0.125 mg/mouse with ASO.

### (Results)

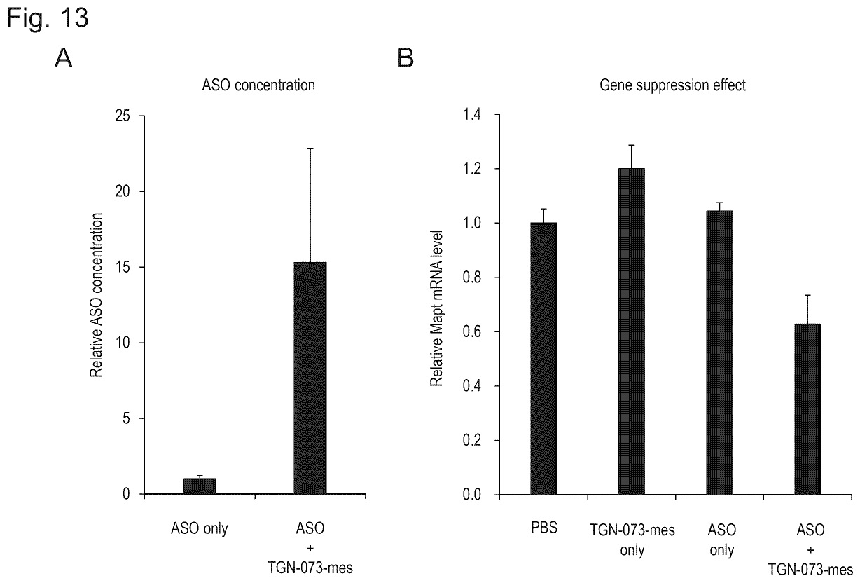

FIG. 13 shows the Mapt RNA expression level in the hippocampus at 7 days after the intraventricular administration. The ASO/TGN-073 coadministration group exhibited a marked enhancement in the ASO concentration in the central nervous system (FIG. 13A) and the target gene suppression effect (FIG. 13B), compared with the ASO single administration group.

### Example 8: Effect of TGN-073 Coadministration in Intraventricular Administration of Single-Stranded PMO

### (Purpose)

TGN-073 is coadministered when a phosphorodiamidate morpholino oligomer (hereinafter referred to as “PMO”) is administered to the cerebral ventricle of a mouse, in which the oligomer is an antisense oligonucleotide that targets the exon 23/intron 23 border region of a dystrophinpre-mRNA for exon skipping. The effect of TGN-073 coadministration on the exon skipping effect in the brain is evaluated.

### (Method)

The base sequence of the PMO used in this Example is shown in Table 7 below.

TABLE 7
Phosphorodiamidate morpholino oligomer (PMO)

| | Sequence (5′-3′) | SEQ ID |
|---|---|---|
| PMO | g•g•c•c•a•a•a•c•c•t•c•g•g•c•t•t•a•c•c•t•g•a•a•a•t | 9 |

Lower case letter (Underlined): morpholino nucleic acid; •: phosphorodiamidate linkage

The PMO described in Table 7 is a 25-mer single-stranded morpholino nucleic acid targeted at the exon 23/intron 23 of pre-mRNA of the mouse dystrophin gene. All of these 25-mer PMO are composed of a morpholino nucleic acid, and all of the internucleoside linkages are phosphorodiamidate linkages. This morpholino nucleic acid has a base sequence complementary with positions 83803536 to 83803512 of dystrophin pre-mRNA (GenBank Accession No. NC000086.7) of a mouse. The PMO was synthesized by GeneDesign Inc. (Osaka, Japan) on consignment.

The mouse used was a 6-week-old male C57BL/6J mouse having a body weight of 20 g. In the following Examples, all the experiments performed using a mouse were based on n=3. PMO (the PMO single administration group) at 2.5 nmol or 2.5 nmol PMO with 250 μg of TGN-073 added thereto (the PMO/TGN-073 coadministration group) was administered in an amount of 10 μL to the left cerebral ventricle of a mouse.

At 1 week after the intraventricular administration, the mouse was dissected, and the hippocampus, striatum, and cortex (occipital lobe) were extirpated. Subsequently, mRNA was extracted from each tissue, using Isogen II. On the total RNA (900 ng) extracted, One-Step RT-PCR was performed using Qiagen One Step RT-PCR Kit (manufactured by Qiagen N.V.). A reaction solution was prepared in accordance with the protocol attached to the Kit. The thermal cycler used was LifeECO (manufactured by Hangzhou Bioer Technology Co., Ltd.). The program used for RT-PCR was as follows: reverse transcription reaction was performed at 42 °C for 30 minutes; then, thermal denaturation was performed at 95 °C for 15 minutes; subsequently, PCR amplification reaction was performed through 35 cycles, in which 1 cycle was performed [at 94 °C for 30 seconds; at 60 °C for 30 seconds; and at 72 °C for 60 seconds]; and the final elongation reaction was performed at 72 °C for 7 minutes. The RT-PCR was performed using the forward (Fw) primer (5′-ATCCAGCAGTCAGAAAGCAAA-3′, SEQ ID NO: 10) and the reverse (Rv) primer having a base sequence (5′-CAGCCATCCATTTCTGTAAGG-3′, SEQ ID NO: 11).

A reaction product of the above-described RT-PCR, in an amount of 1 μL, was analyzed using Bioanalyzer 2100 (manufactured by Agilent Technologies, Inc.), and using an Agilent DNA1000 kit. The number “A” of moles of the nucleotide of the band skipped by exon 23 and the number “B” of moles of the nucleotide of the band not skipped by exon 23 were measured. On the basis of the measurement values of these “A” and “B”, the skipping efficiency was determined in accordance with the following formula: skipping efficiency (%)=A/(A+B)×100.

### (Results)

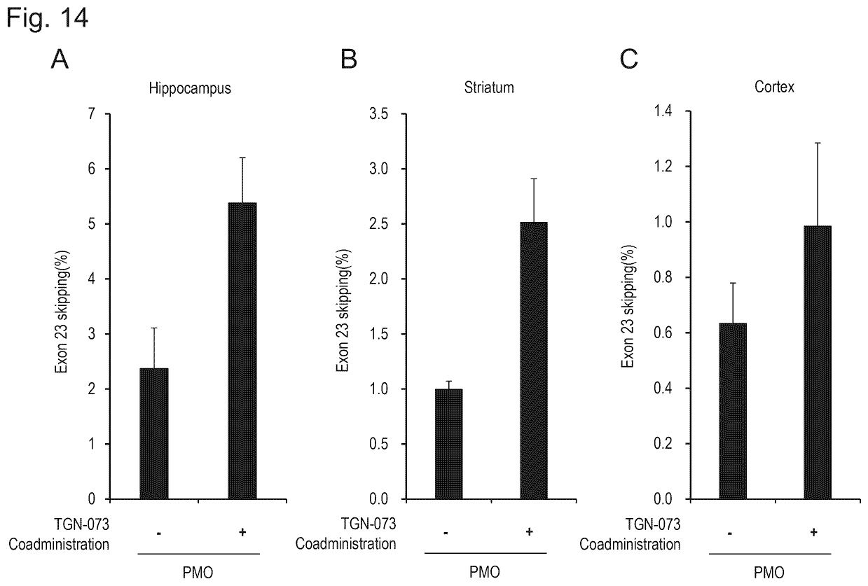

FIG. 14 shows the Exon 23 skipping efficiency in the hippocampus (FIG. 14A), striatum (FIG. 14B), and cortex (FIG. 14C) at 7 days after the intraventricular administration. In any site in the brain, the PMO/TGN-073 coadministration group exhibited a noticeable increase in the skipping efficiency, compared with the PMO single administration group.

### Example 9: Effect of TGN-073 Coadministration on Intraventricular Administration of Double-Stranded Nucleic Acid Complex Comprising PMO

### (Purpose)

TGN-073 is coadministered in the intraventricular administration of a double-stranded nucleic acid complex to a mouse, in which the complex consists of: a PMO that targets the exon 23/intron 23 border region of dystrophinpre-mRNA for exon skipping; and a complementary strand comprising a sequence complementary to the PMO, and bound to cholesterol (the complex is hereinafter referred to as “Chol-HDO (PMO)”). The effect of TGN-073 coadministration on the exon skipping effect in the brain is evaluated.

### (Method)

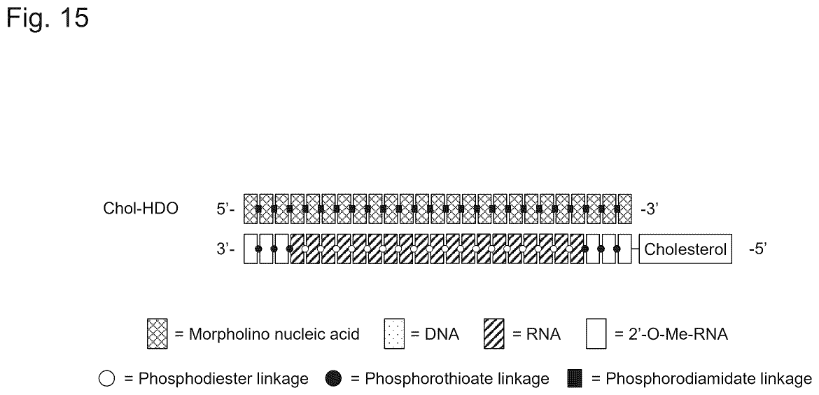

The base sequences of the PMO and the complementary strand that were used in this Example are shown in Table 8 below and FIG. 15.

TABLE 8
Chol-HDO(PMO)

| | | Sequence (5′-3′) | SEQ ID NO |
|---|---|---|---|
| Chol-HDO | PMO | g•g•c•c•a•a•a•c•c•t•c•g•g•c•t•t•a•c•c•t•g•a•a•a•t | 9 |
| (PMO) | Chol- | Chol-A(M)^{∧}U(M)^{∧}U(M)^{∧}U*C*A*G*G*U*A*A*G*C* | 12 |
| | cRNA | C*G*A*G*G*U*U*U*G^{∧}G(M)^{∧}C(M)^{∧}C(M) | |

Lower case letter (Underlined): morpholino nucleic acid; Upper case letter: RNA; Upper case letter (M): 2′-O-Me-RNA; •: phosphorodiamidate linkage; ^{∧}: phosphorothioate linkage; *: phosphorothioate linkage; Chol: cholesterol

Chol-HDO (PMO) described in Table 8 is composed of PMO and Chol-cRNA. PMO is the same single-stranded morpholino nucleic acid as in Example 8. Chol-cRNA has a base sequence complementary to the above-described PMO, and has a structure in which cholesterol is bound to the 5′ side, and in which three 2′-O-methyl-modified RNA nucleosides at the 5′ end, three 2′-O-methyl-modified RNA nucleosides at the 3′ end, and 19 DNA nucleosides therebetween are linked via a phosphorothioate linkage and a phosphodiester linkage. PMO and Chol-cRNA were synthesized by GeneDesign Inc. (Osaka, Japan) on consignment, and both were annealed to prepare a double-stranded Chol-HDO. Specifically, PMO and Chol-cRNA were mixed in equimolar amounts, and the resulting solution was heated at 95 °C for 5 minutes, then cooled to 37 °C, and maintained for 1 hour, allowing the nucleic acid strands to be annealed to thereby prepare the above-described double-stranded nucleic acid agent.

In accordance with the method in Example 8, Chol-HDO (PMO)(the Chol-HDO (PMO) single administration group) at 2.5 nmol or 2.5 nmol Chol-HDO (PMO) having 250 μg of TGN-073 added thereto (the Chol-HDO (PMO)/TGN-073 coadministration group) was administered in an amount of 10 μL to the left cerebral ventricle of a mouse. The skipping efficiency was thus evaluated.

### (Results)

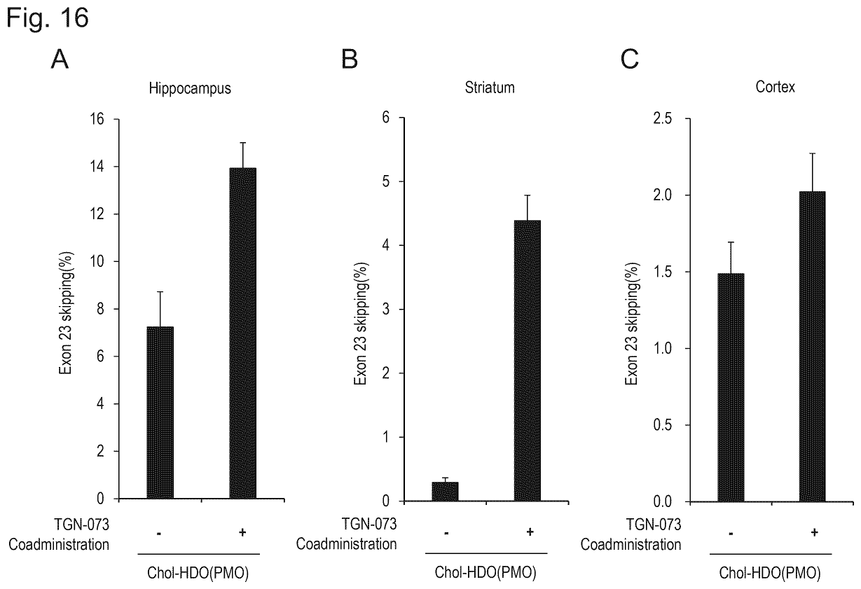

FIG. 16 shows the Exon 23 skipping efficiency in the hippocampus (FIG. 16A), striatum (FIG. 16B), and cortex (FIG. 16C) at 7 days after the intraventricular administration. In any site in the brain, the Chol-HDO (PMO)/TGN-073 coadministration group exhibited a noticeable increase in the skipping efficiency, compared with the Cho-HDO (PMO) single administration group.

### Example 10: Effect of TGN-073 Coadministration on Delivery of Nucleic Acid Strand to Spinal Cord

### (Purpose)

The Malat1-targeting ASO used in Example 1, together with TGN-073, is administered to the cerebral ventricle. Investigated through an in vivo experiment are: the influences of the TGN-073 coadministration on the target gene suppression effect in a spinal cord and on the amount of the nucleic acid delivered to the spinal cord.

### (Method)

The ASO used in this Example was a Malat1-targeting ASO described in Example 1. Specifically, on the ASO described in Table 1, the preparation of the nucleic acid, an in vivo experiment, and the evaluation of the target gene suppression effect were performed by the same methods as in Example 1. In this Example, however, the amount of the nucleic acid agent administered per mouse was 1.7 nmol (12.5 μg)/mouse. The amount of delivery of the nucleic acid and the target gene suppression effect were evaluated in the lumbar cord portion.

### (Results)

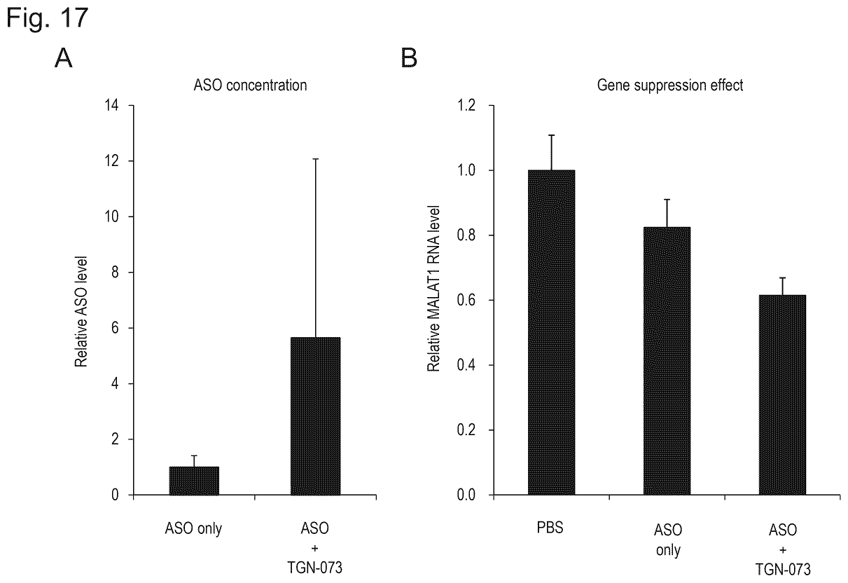

FIG. 17A shows the ASO concentration in the lumbar cord portion at 7 days after the intraventricular administration. The ASO/TGN-073 coadministration group exhibited a dramatic increase in the ASO concentration in the lumbar cord portion, compared with the ASO single administration group.

FIG. 17B shows the Malat1 RNA expression level in the lumbar cord portion at 7 days after the intraventricular administration. The ASO/TGN-073 coadministration group exhibited a marked enhancement in the target gene suppression effect in the lumbar cord portion, compared with the ASO single administration group.

The above-described results have revealed that the TGN-073 coadministration exhibited a dramatic enhancement in the efficiency of delivery of the nucleic acid to the central nervous system comprising a brain tissue and a spinal cord, and the target gene suppression effect in the central nervous system comprising a brain tissue and a spinal cord.

### Example 11: Effect of TGN-073 Coadministration in Intrathecal Administration of ASO

### (Purpose)

The effect of TGN-073 coadministration is investigated in reference to intrathecal administration that is non-intraventricular administration. Specifically, the Mapt-targeting ASO used in Example 3 was administered intrathecally, and the influence of TGN-073 coadministration on the amount of the nucleic acid delivered to the spinal cord tissue and the hippocampus is investigated through an in vivo experiment.

### (Method)

In this Example, an ASO having the same configuration as the ASO used in Example 3 (the ASO described in Table 2) was used, and a nucleic acid was prepared by the same method as in Example 1.

The intrathecal administration of ASO and the measurement of the ASO concentration were performed by the following methods. A catheter was inserted into the spinal subarachnoid of an 8-week-old Slc:SD rat (male). When the rat was 9 weeks old, ASO and/or TGN-073 in an amount of 76 nmol (400 μg) per rat (1.5 g/rat) was administered intrathecally. Hereinafter, the rats to which ASO alone was intrathecally administered are referred to as the “ASO single administration group”, and the rats to which ASO and TGN-073 were intrathecally administered together are referred to as the “ASO/TGN-073 coadministration group”. The spinal part and the left hippocampus were extirpated from the rats at 7 days after the intrathecal administration, and the ASO concentration in each tissue was evaluated by the same method as in Example 1.

### (Results)

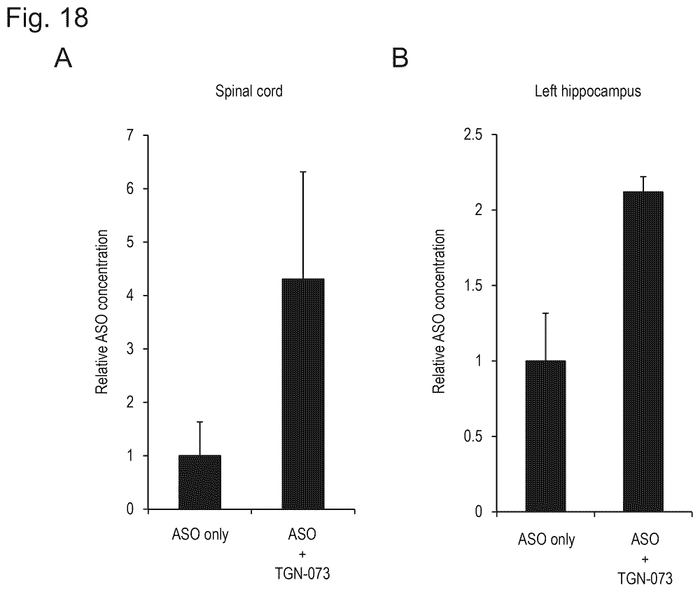

FIG. 18 shows the ASO concentration in the spinal cord tissue (FIG. 18A) and the left hippocampus (FIG. 18B) at 7 days after the intrathecal administration. The ASO/TGN-073 coadministration group exhibited a dramatic increase in the ASO concentration in the spinal part and the left hippocampus, compared with the ASO single administration group.

The above-described results have revealed that, not only in intraventricular administration but also in intrathecal administration, the TGN-073 coadministration was able to dramatically enhance the efficiency of delivery of the nucleic acid to the central nervous system.

### Example 12: Effect of TGN-073 Coadministration in Intraventricular Administration of VHH Antibody

### (Purpose)

Through a mouse in vivo experiment, the influence of the TGN-073 coadministration in the intraventricular administration of a VHH antibody is investigated in order to the investigate the effect of the TGN-073 coadministration on a medicament modality other than of a nucleic acid medicine.

### (Method)

In this Example, a fluorescence-labeled camelized single domain antibody (VHH antibody; Alpaca anti-Rabbit IgG Nano (VHH) Recombinant Secondary Antibody, Alexa Fluor (trademark)₆₄₇, #SA5-10327, Invitrogen) alone or in combination with TGN-073 (250 μg/mouse) was intraventricularly administered at a dose of 29 μmol/mouse to an 8-week-old ICR mouse (female). Hereinafter, the mice to which the VHH antibody alone was intraventricularly administered are referred to as the “VHH antibody single administration group”, and the mice to which the VHH antibody and TGN-073 were intraventricularly administered together are referred to as the “VHH antibody/TGN-073 coadministration group”. Brain tissue samples of the left hippocampus, left occipital cortex, and left basal ganglia were collected from the mice at 3 hours after the intraventricular administration. Using a fluorescence plate reader (M1000Pro, Tecan Group Ltd.), the Alexa 647 fluorescence intensity per brain weight was measured to thereby evaluate the concentration of the VHH antibody in each brain tissue.

### (Results)

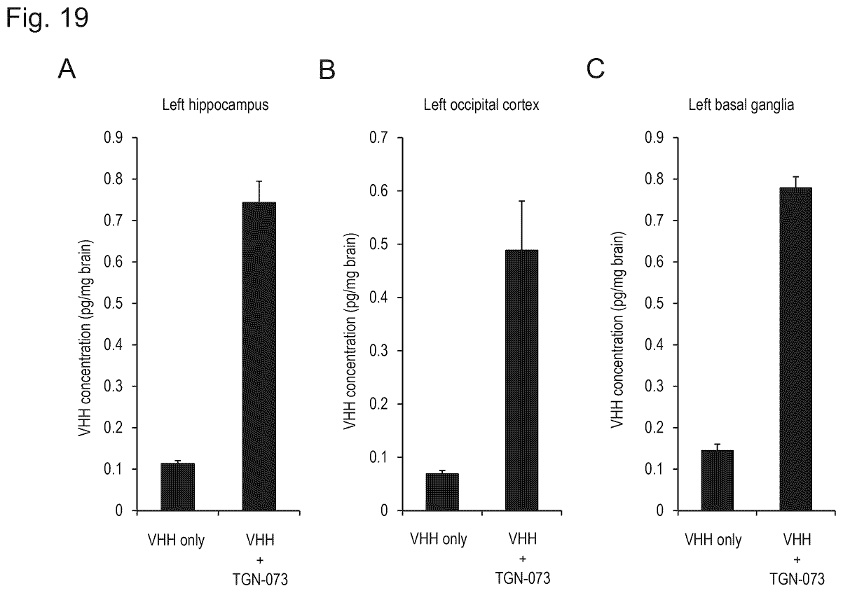

FIG. 19 shows the VHH antibody concentration in each of the left hippocampus (FIG. 19A), left occipital cortex (FIG. 19B), and left basal ganglia (FIG. 19C) after the intraventricular administration. The VHH antibody/TGN-073 coadministration group exhibited a dramatic increase in the VHH antibody concentration in the central nervous system, compared with the VHH antibody single administration group.

The above-described results have revealed that not only the nucleic acid medicine but also the VHH antibody, coadministered with TGN-073, was able to dramatically enhance the efficiency of delivery to the central nerve when intraventricularly administered.

### Example 13: Effect of TGN-073 Coadministration in Intraventricular Administration of IgG Antibody

### (Purpose)

In reference to delivery of an IgG antibody, the efficacy of TGN-073 coadministration to a mouse is investigated through an in vivo experiment.

### (Method)

In this Example, an Alexa 647 fluorescence-labeled IgG antibody (Goat anti-Rabbit IgG (H+L) Highly Cross-Adsorbed Secondary Antibody, Alexa Fluor (trademark)₆₄₇, #A-21245, Invitrogen) alone or in combination with TGN-073 (250 μg/mouse) was administered at a dose of 10 μg/mouse to the left lateral ventricle of an 8-week-old ICR mouse (female). Hereinafter, the mice to which the IgG antibody alone was intraventricularly administered are referred to as the “IgG antibody single administration group”, and the mice to which the IgG antibody and TGN-073 were intraventricularly administered together are referred to as the “IgG/TGN-073 coadministration group”. Brain tissue samples of the left hippocampus were collected from the mice at 3 hours after the intraventricular administration. Using a fluorescence plate reader (M1000Pro, Tecan Group Ltd.), the Alexa 647 fluorescence intensity per brain weight was measured to thereby evaluate the concentration of the IgG antibody.

### (Results)

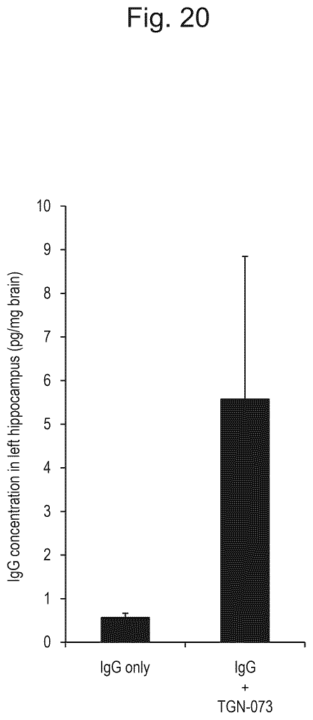

FIG. 20 shows the IgG antibody concentration in the left hippocampus after the intraventricular administration. The IgG/TGN-073 coadministration group exhibited a dramatic increase in the IgG antibody concentration in the central nervous system, compared with the IgG antibody single administration group.

The above-described results have revealed that the IgG antibody, coadministered with TGN-073, was also able to dramatically enhance the efficiency of delivery to the central nerve when intraventricularly administered.

### Example 14: Effect of TGN-073 Coadministration in Intraventricular Administration of Viral Vector

### (Purpose)

Investigated through a mouse in vivo experiment is the influence of TGN-073 coadministration on the intraventricular administration of an AAV gene-therapeutic drug.

### (Method)

In this Example, a GFP expression AAV9 (AAV9-CMV-hrGFP) as an adeno-associated viral (AAV) vector, alone or in combination with TGN-073 (250 μg/mouse), was administered at a dose of 26× 109 vg/mouse to the left lateral ventricle of a 7-week-old ICR mouse (female). Hereinafter, the mice to which only the AAV vector was intraventricularly administered are referred to as the “AAV single administration group”, and the mice to which the AAV vector and TGN-073 were intraventricularly administered together are referred to as the “AAV/TGN-073 coadministration group”. At 3 weeks after the intraventricular administration, the mouse was subjected to perfusion fixation using 4% paraformaldehyde. The mouse was subjected to cryoprotective treatment using a solution of 10% to 30% sucrose dissolved in PBS, and then, a cut-piece having a thickness of 40 μm was produced using a microtome. Using a fluorescence microscope (VHS-7000, Keyence Corporation), the GFP fluorescence (hippocampus-level coronal image) was captured.

### (Results)

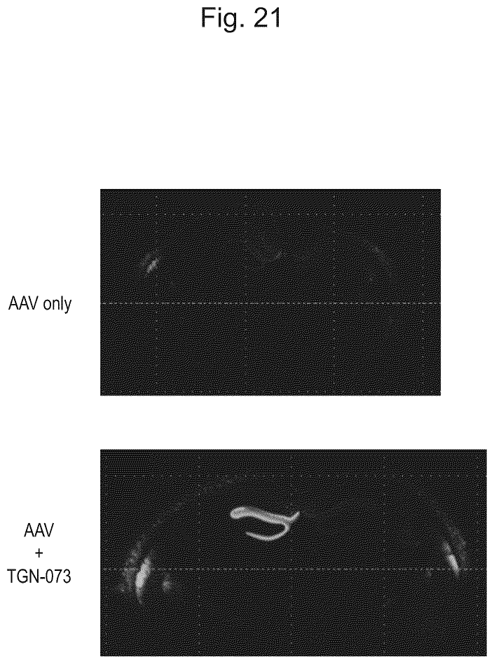

FIG. 21 illustrates the coronal images at the hippocampus level at 3 weeks after the intraventricular administration. The AAV/TGN-073 coadministration group allowed the GFP expression to be observed in a significantly wide range in the central nervous system, and exhibited an increase in the expression amount, compared with the AAV single administration group.

The above-described results have revealed that, also in the intraventricular administration of the viral vector, the TGN-073 coadministration was able to dramatically enhance the efficiency of delivery to the central nerve.

All publications, patents, and patent applications cited herein are incorporated herein by reference in their entirety.

1. A method of promoting delivery of a drug to the central nervous system and/or the peripheral nervous system by administering an agent for promoting the function of Aquaporin-4 or an agent for modifying the function of Aquaporin-4.

2. The method of claim 1, wherein the agent for promoting the function of Aquaporin-4 or the agent for modifying the function of Aquaporin-4 is a compound represented by the following formula (I) or a derivative thereof, or a salt of any of the compound and the salt;

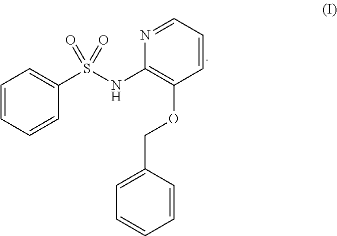 [SMILES: O=S(=O)(Nc1ncccc1OCc1ccccc1)c1ccccc1]

3. The method of claim 2, wherein the derivative is represented by the following formula (III):

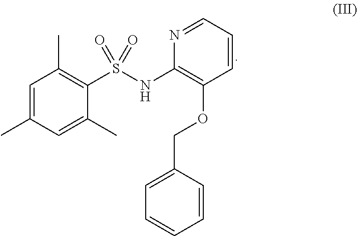 [SMILES: Cc1cc(C)c(S(=O)(=O)Nc2ncccc2OCc2ccccc2)c(C)c1]

4-8. (canceled)

9. The method of claim 1, wherein the drug is administered by intrathecal administration, transnasal administration, intravenous administration, subcutaneous administration, intraperitoneal administration, oral administration, inhalation administration, or intramuscular administration.

10. The method of claim 9, wherein the intrathecal administration is intraventricular administration, suboccipital puncture, or lumbar puncture.

11. The method of claim 1, wherein the drug is administered with a shunt, indwelling catheter, or subcutaneous port.

12. The method of claim 1, wherein the agent is administered simultaneously with the drug, or administered before or after the drug.

13. The method of claim 1, wherein the agent is administered by intrathecal administration, transnasal administration, intravenous administration, subcutaneous administration, intraperitoneal administration, oral administration, inhalation administration, or intramuscular administration.

14. A method of treating the central nervous system disease or the peripheral nervous system disease, comprising administering a therapeutically effective amount of a drug, and administering an agent for promoting the function of Aquaporin-4 or an agent for modifying the function of Aquaporin-4.

15. The method of claim 14, wherein the drug and/or the agent is administered by intrathecal administration, transnasal administration, intravenous administration, subcutaneous administration, intraperitoneal administration, oral administration, inhalation administration, or intramuscular administration.

16. The method of claim 15, wherein the intrathecal administration is intraventricular administration, suboccipital puncture, or lumbar puncture.

17. (canceled)

18. The method of claim 1, wherein the drug is a nucleic acid medicine, peptide, small molecule compound, viral vector, cellular medicine, nanoparticle, liposome, micelle, or exosome.

19-21. (canceled)

22. The method of claim 18, wherein the peptide is an antibody or an antibody fragment.

23-24. (canceled)

25. The method of claim 18, wherein the nucleic acid medicine is selected from the group consisting of an antisense nucleic acid, heteronucleic acid, siRNA, shRNA, miRNA, mRNA, lncRNA, plasmid DNA, aptamer, decoy, bait nucleic acid, ribozyme, and nucleic acid vector.

26. The method of claim 18, wherein the nucleic acid medicine comprises a nucleic acid molecule which is capable of hybridizing to at least part of a target gene or a transcriptional product thereof, and has an antisense effect for the target gene or the transcriptional product thereof.

27-30. (canceled)

31. The method of claim 26, wherein the nucleic acid molecule is a gapmer or a mixmer.

32-62. (canceled)

63. The method of claim 18, wherein the nucleic acid medicine is siRNA.

64-69. (canceled)

70. The method of claim 1, wherein the central nervous system is selected from the group consisting of cerebral cortex, basal ganglia, cerebral white matter, diencephalon, brainstem, cerebellum, and spinal cord.

71. (canceled)

72. The method of claim 1, wherein the peripheral nervous system is selected from the group consisting of a ventral root of the spinal cord, dorsal root, 1st to 12th cranial nerves, cauda equina, and dorsal root ganglion.

73. The method of claim 1, wherein 0.01 mg to 10 g of the agent for promoting delivery is administered to a subject.
